SEQUENCE LISTING (37 sequences)

SEQ ID NO: 1 - protein, 8 aa, synthetic construct
GYTFSRYW

SEQ ID NO: 2 - protein, 8 aa, synthetic construct
ILPGSGST

SEQ ID NO: 3 - protein, 11 aa, synthetic construct
TEGYEYDGFDY

SEQ ID NO: 4 - protein, 11 aa, synthetic construct
QSIVYSNGNTY

SEQ ID NO: 5 - protein, 9 aa, synthetic construct
FQGSHIPYT

SEQ ID NO: 6 - protein, 219 aa, synthetic construct
QVQLQQSGAELMKPGASVKISCKATGYTFSRYWIEWVKQRPGHGLEWIGEILPGSGSTNYNEKFKGKATITADTSSNTAYMQLSSLTSEDSAVYYCTEGYEYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 7 - protein, 219 aa, synthetic construct
QVQLVQSGAEVKKPGSSVKVSCKASGYTFSRYWISWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 8 - protein, 219 aa, synthetic construct
QVQLVQSGAEVKKPGSSVKVSCKASGYTFSRYWIEWVRQAPGQGLEWMGRILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 9 - protein, 219 aa, synthetic construct
QVQLVQSGAEVKKPGSSVKVSCKATGYTFSRYWISWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 10 - protein, 219 aa, synthetic construct
QVQLVQSGAEVKKPGSSVKVSCKATGYTFSRYWIEWVRQAPGQGLEWMGEILPGSGSTNYAQKFQGRVTITADESTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPK

SEQ ID NO: 11 - protein, 219 aa, synthetic construct
DVLLSQTPLSLPVSLGDQATISCRSSQSIVYSNGNTYLEWYLQKPGQSPKLLIYRVSNRFSGVPDRFSGSGSGTDFTLKISRVEAEDLGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 219 aa, synthetic construct
DVVMTQSPLSLPVTLGQPASISCRSSQSIVYSNGNTYLNWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 219 aa, synthetic construct
DVVMTQSPLSLPVTLGQPASISCRSSQSIVYSNGNTYLEWFQQRPGQSPRRLIYRVSNRDSGVPDRFSGSGSGTDFTLKISRVEAEDVGVYYCFQGSHIPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 21 aa, Homo sapiens
YRQSMNNFQGLRSFGCRFGTC

SEQ ID NO: 15 - protein, 12 aa, Homo sapiens
CTVQKLAHQIYQ

SEQ ID NO: 16 - protein, 12 aa, Homo sapiens
  MOD_RES 12: note = C-terminal amidation
CAPRSKISPQGY

SEQ ID NO: 17 - protein, 19 aa, Mus musculus
YRQSMNQGSRSNGCRFGTC

SEQ ID NO: 18 - protein, 12 aa, Mus musculus
CTFQKLAHQIYQ

SEQ ID NO: 19 - protein, 12 aa, Mus musculus
  MOD_RES 12: note = C-terminal amidation
CAPRNKISPQGY

SEQ ID NO: 20 - protein, 52 aa, Homo sapiens
  MOD_RES 52: note = C-terminal amidation
YRQSMNNFQGLRSFGCRFGTCTVQKLAHQIYQFTDKDKDNVAPRSKISPQGY

SEQ ID NO: 21 - protein, 50 aa, Mus musculus
  MOD_RES 50: note = C-terminal amidation
YRQSMNQGSRSNGCRFGTCTFQKLAHQIYQLTDKDKDGMAPRNKISPQGY

SEQ ID NO: 22 - protein, 21 aa, Homo sapiens
YRQSMNNFQGLRSFGCRFGTC

SEQ ID NO: 23 - protein, 42 aa, Homo sapiens
YRQSMNNFQGLRSFGCRFGTCTVQKLAHQIYQFTDKDKDNVA

SEQ ID NO: 24 - protein, 10 aa, Homo sapiens
  MOD_RES 10: note = C-terminal amidation
PRSKISPQGY

SEQ ID NO: 25 - protein, 14 aa, Homo sapiens
YRQSMNNFQGLRSF

SEQ ID NO: 26 - protein, 10 aa, Homo sapiens
YRQSMNNFQG

SEQ ID NO: 27 - protein, 6 aa, Homo sapiens
YRQSMN

SEQ ID NO: 28 - protein, 32 aa, Homo sapiens
YRQSMNNFQGLRSFGCRFGTCTVQKLAHQIYQ

SEQ ID NO: 29 - protein, 40 aa, Mus musculus
YRQSMNQGSRSNGCRFGTCTFQKLAHQIYQLTDKDKDGMA

SEQ ID NO: 30 - protein, 31 aa, Mus musculus
YRQSMNQGSRSNGCRFGTCTFQKLAHQIYQL

SEQ ID NO: 31 - protein, 164 aa, Homo sapiens
ARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 32 - protein, 448 aa, synthetic construct
QVQLVQSGAEVKKPGSSVKVSCKASGYTFSRYWIEWVRQAPGQGLEWIGEILPGSGSTNYNQKFQGRVTITADTSTSTAYMELSSLRSEDTAVYYCTEGYEYDGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 219 aa, synthetic construct
DVVLTQSPLSLPVTLGQPASISCRSSQSIVYSNGNTYLEWYLQRPGQSPRLLIYRVSNRFSGVPDRFSGSGSGTDFTLKISRVEAEDVGVYYCFQGSHIPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 737 aa, Homo sapiens
MADTQYILPNDIGVSSLDCREAFRLLSPTERLYAYHLSRAAWYGGLAVLLQTSPEAPYIYALLSRLFRAQDPDQLRQHALAEGLTEEEYQAFLVYAAGVYSNMGNYKSFGDTKFVPNLPKEKLERVILGSEAAQQHPEEVRGLWQTCGELMFSLEPRLRHLGLGKEGITTYFSGNCTMEDAKLAQDFLDSQNLSAYNTRLFKEVDGEGKPYYEVRLASVLGSEPSLDSEVTSKLKSYEFRGSPFQVTRGDYAPILQKVVEQLEKAKAYAANSHQGQMLAQYIESFTQGSIEAHKRGSRFWIQDKGPIVESYIGFIESYRDPFGSRGEFEGFVAVVNKAMSAKFERLVASAEQLLKELPWPPTFEKDKFLTPDFTSLDVLTFAGSGIPAGINIPNYDDLRQTEGFKNVSLGNVLAVAYATQREKLTFLEEDDKDLYILWKGPSFDVQVGLHELLGHGSGKLFVQDEKGAFNFDQETVINPETGEQIQSWYRSGETWDSKFSTIASSYEECRAESVGLYLCLHPQVLEIFGFEGADAEDVIYVNWLNMVRAGLLALEFYTPEAFNWRQAHMQARFVILRVLLEAGEGLVTITPTTGSDGRPDARVRLDRSKIRSVGKPALERFLRRLQVLKSTGDVAGGRALYEGYATVTDAPPECFLTLRDTVLLRKESRKLIVQPNTRLEGSDVQLLEYEASAAGLIRSFSERFPEDGPELEEILTQLATADARFWKGPSEAPSGQA

SEQ ID NO: 35 - protein, 21 aa, Homo sapiens
CETVINPETGEQIQSWYRSGE

SEQ ID NO: 36 - protein, 118 aa, synthetic construct
QVQLVQSGAEVKKPGSSVKVSCKASGGTFSSYAISWVRQAPGQGLEWMGRIIPILGTANYAQKFQGRVTITADESTSTAYMELSSLRSEDTAVYYCARYYYYYGMDVWGQGTTVTVSS

SEQ ID NO: 37 - protein, 118 aa, synthetic construct
QVQLQQSGAELMKPGASVKISCKATGYTFSRYWIEWVKQRPGHGLEWIGEILPGSGSTNYNEKFKGKATITADTSSNTAYMQLSSLTSEDSAVYYCTEGYEYDGFDYWGQGTTLTVSS

(19) United States
(12) Patent Application Publication
(10) Pub. No.: US 2026/0167706 A1
(43) Pub. Date: Jun. 18, 2026

(54) ANTI-ADRENOMEDULLIN (ADM) ANTIBODY OR ANTI-ADM ANTIBODY FRAGMENT OR ANTI-ADM NON-IG SCAFFOLD FOR USE IN THERAPY OR PREVENTION OF SHOCK

(71) Applicant: ADRENOMED AG, Hennigsdorf (DE)

(72) Inventor: Andreas BERGMANN, Berlin (DE)

(73) Assignee: ADRENOMED AG, Hennigsdorf (DE)

(21) Appl. No.: 19/099,348

(22) PCT Filed: Jul. 31, 2023
(86) PCT No.: PCT/EP2023/071215

§ 371 Date: Jan. 28, 2025

(30) Foreign Application Priority Data
Jul. 29, 2022 (EP) 22187954.7

(51) Int. Cl. C07K 16/22; A61P 37/06

(52) U.S. Cl. CPC C07K 16/22; A61P 37/06; C07K 2317/24; C07K 2317/76; C07K 2317/92

(57) ABSTRACT

The present application is directed to an anti-Adrenomedullin (ADM) antibody or anti-ADM antibody fragment or anti-ADM non-Ig scaffold for use in the treatment or prevention of shock in a patient, wherein said patient is characterized by having a level of dipeptidyl peptidase 3 (DPP3) in a sample of bodily fluid below a threshold, which is ≤40 ng/ml or in the range between 22 and 40 ng/ml and said anti-ADM antibody or anti-ADM fragment or anti-ADM non-Ig scaffold binds to the N-terminal part (amino acid 1-21) of ADM: YRQSMNNFQGLRSFGCRFGTC (SEQ ID No. 14).

### FIELD OF THE INVENTION

Subject matter of the present invention is an anti-Adrenomedullin (ADM) antibody or anti-ADM antibody fragment or anti-ADM non-Ig scaffold for use in therapy or prevention of shock in a patient, wherein said patient is characterized by having a level of dipeptidyl peptidase 3 (DPP3) in a sample of bodily fluid below a threshold, which is ≤40 ng/ml or in the range between 22 and 40 ng/ml and said anti-ADM antibody or anti-ADM fragment or anti-ADM non-Ig scaffold binds to the N-terminal part (amino acid 1-21) of ADM: YRQSMNNFQGLRSFGCRFGTC (SEQ ID No. 14).

### BACKGROUND

Dipeptidyl peptidase 3—also known as Dipeptidyl aminopeptidase III, Dipeptidyl arylamidase III, Dipeptidyl peptidase III, Enkephalinase B or red cell angiotensinase; short name: DPP3, DPPIII—is a metallopeptidase that removes dipeptides from physiologically active peptides, such as enkephalins and angiotensins. DPP3 was first identified and its activity measured in extracts of purified bovine anterior pituitary by Ellis & Nuenke 1967. The enzyme, which is listed as EC 3.4.14.4, has a molecular mass of about 83 kDa and is highly conserved in procaryotes and eucaryotes (Prajapati & Chauhan 2011). The amino acid sequence of the human variant is depicted in SEQ ID NO 1. Dipeptidyl peptidase III is a mainly cytosolic peptidase which is ubiquitously expressed. Despite lacking a signal sequence, a few studies reported membranous activity (Lee & Snyder 1982).

DPP3 is a zinc-depending exo-peptidase belonging to the peptidase family M49. It has a broad substrate specificity for oligopeptides from three/four to ten amino acids of various compositions and is also capable of cleaving after proline. DPP3 is known to hydrolyze dipeptides from the N-terminus of its substrates, including angiotensin II, III and IV; Leu- and Met-enkephalin; endomorphin 1 and 2. The metallopeptidase DPP3 has its activity optimum at pH 8.0-9.0 and can be activated by addition of divalent metal ions, such as Co²⁺ and Mg²⁺.

Structural analysis of DPP3 revealed the catalytic motifs HELLGH (hDPP3 450-455) and EECRAE (hDPP3 507-512), as well as following amino acids, that are important for substrate binding and hydrolysis: Glu316, Tyr, 318, Asp366, Asn391, Asn394, His568, Arg572, Arg577, Lys666 and Arg669 (Prajapati & Chauhan 2011; Kumar et al. 2016; numbering refers to the sequence of human DPP3, see SEQ ID NO. 1). Considering all known amino acids or sequence regions that are involved in substrate binding and hydrolysis, the active site of human DPP3 can be defined as the area between amino acids 316 and 669.

The most prominent substrate of DPP3 is angiotensin II (Ang II), the main effector of the renin-angiotensin system (RAS). The RAS is activated in cardiovascular diseases (Dostal et al. 1997. J Mol Cell Cardiol; 29:2893-902; Roks et al. 1997. Heart Vessels. Suppl 12:119-24), sepsis, and septic shock (Corra et al. 2015. Crit Care 2015:19:98). In particular, Ang II, has been shown to modulate many cardiovascular functions including the control of blood pressure and cardiac remodeling.

Recently, two assays were generated, characterized, and validated to specifically detect DPP3 in human bodily fluids (e.g., blood, plasma, serum): a luminescence immunoassay (LIA) to detect DPP3 protein concentration and an enzyme capture activity assay (ECA) to detect specific DPP3 activity (Rehfeld et al. 2019. JALM 3(6): 943-953). A washing step removes all interfering substances before the actual detection of DPP3 activity is performed. Both methods are highly specific and allow the reproducible detection of DPP3 in blood samples.

Circulating DPP3 levels were shown to be increased in cardiogenic shock patients and were associated with an increased risk of short-term mortality and severe organ dysfunction (Deaniau et al. 2019. Eur J Heart Fail. in press). Moreover, DPP3 measured at inclusion discriminated cardiogenic shock patients who did develop refractory shock vs. non-refractory shock and a DPP3 concentration ≥59.1 ng/mL was associated with a greater risk of death (Takagi et al. Eur J Heart Fail. in press).

The peptide adrenomedullin (ADM) was described for the first time in 1993 (Kitamura et al., 1993. Biochem Biophys Res Comm 192 (2): 553-560) as a novel hypotensive peptide comprising 52 amino acids, which had been isolated from a human pheochromocytoma cell line (SEQ ID No.: 20). In the same year, cDNA coding for a precursor peptide comprising 185 amino acids and the complete amino acid sequence of this precursor peptide were also described. The precursor peptide, which comprises, inter alia, a signal sequence of 21 amino acids at the N-terminus, is referred to as “pre-proadrenomedullin” (pre-proADM). In the present description, all amino acid positions specified usually relate to the pre-proADM, which comprises the 185 amino acids. The peptide adrenomedullin (ADM) is a peptide which comprises 52 amino acids (SEQ ID No: 20) and which comprises the amino acids 95 to 146 of pre-proADM, from which it is formed by proteolytic cleavage. To date, substantially only a few fragments of the peptide fragments formed in the cleavage of the pre-proADM have been more exactly investigated, in particular the physiologically active peptides ADM and “PAMP”, a peptide comprising 20 amino acids (22-41), which follows the 21 amino acids of the signal peptide in pre-proADM. The discovery and characterization of ADM in 1993 triggered intensive research activity, the results of which have been summarized in various review articles, in the context of the present description, reference being made in particular to the articles to be found in an issue of “Peptides” devoted to ADM in particular (Takahashi 2001. Peptides 22: 1691; Eto 2001. Peptides 22: 1693-1711). A further review is Hinson et al. 2000 (Hinson et al. 2000. Endocrine Reviews 21(2):138-167). In the scientific investigations to date, it has been found, inter alia, that ADM may be regarded as a polyfunctional regulatory peptide. It is released into the circulation in an inactive form extended by glycine (Kitamura et al. 1998. Biochem Biophys Res Commun 244(2): 551-555). There is also a binding protein (Pio et al. 2001. The Journal of Biological Chemistry 276(15): 12292-12300), which is specific for ADM and probably likewise modulates the effect of ADM. Those physiological effects of ADM as well as of PAMP, which are of primary importance in the investigations to date, were the effects influencing blood pressure.

Hence, ADM is an effective vasodilator, and thus it is possible to associate the hypotensive effect with the particular peptide segments in the C-terminal part of ADM. It has furthermore been found that the above-mentioned physiologically active peptide PAMP formed from pre-proADM likewise exhibits a hypotensive effect, even if it appears to have an action mechanism differing from that of ADM (in addition to the above mentioned review articles Eto et al. 2001 and Hinson et al. 2000 see also Kuwasaki et al. 1997. FEBS Lett 414(1): 105-110; Kuwasaki et al. 1999. Ann. Clin. Biochem. 36: 622-628; Tsuruda et al. 2001 Life Sci. 69(2): 239-245 and EP-A2 0 622 458). It has furthermore been found, that the concentrations of ADM, which can be measured in the circulation and other biological liquids, are in a number of pathological states, significantly above the concentrations found in healthy control subjects. Thus, the ADM level in patients with congestive heart failure, myocardial infarction, kidney diseases, hypertensive disorders, diabetes mellitus, in the acute phase of shock and in sepsis and septic shock are significantly increased, although to different extents. The PAMP concentrations are also increased in some of said pathological states, but the plasma levels are lower relative to ADM (Eto 2001. Peptides 22: 1693-1711). It was reported that unusually high concentrations of ADM are observed in sepsis, and the highest concentrations in septic shock (Eto 2001. Peptides 22: 1693-1711; Hirata et al. Journal of Clinical Endocrinology and Metabolism 81(4): 1449-1453; Ehlenz et al. 1997. Exp Clin Endocrinol Diabetes 105: 156-162; Tomoda et al. 2001. Peptides 22: 1783-1794; Ueda et al. 1999. Am. J. Respir. Crit. Care Med. 160: 132-136 and Wang et al. 2001. Peptides 22: 1835-1840).

Plasma concentrations of ADM are elevated in patients with heart failure and correlate with disease severity (Hiravama et al. 1999. J Endocrinol 160: 297-303; Yu et al. 2001. Heart 86: 155-160). High plasma ADM is an independent negative prognostic indicator in these subjects (Povner et al. 2002. Pharmacol Rev 54: 233-246).

WO2004/097423 describes the use of an antibody against adrenomedullin for diagnosis, prognosis, and treatment of cardiovascular disorders. Treatment of diseases by blocking the ADM receptor are also described in the art, (e.g. WO2006/027147, PCT/EP2005/012844) said diseases may be sepsis, septic shock, cardiovascular diseases, infections, dermatological diseases, endocrinological diseases, metabolic diseases, gastroenterological diseases, cancer, inflammation, hematological diseases, respiratory diseases, muscle skeleton diseases, neurological diseases, urological diseases.

It is reported for the early phase of sepsis that ADM improves heart function and the blood supply in liver, spleen, kidney and small intestine. Anti-ADM-neutralizing antibodies neutralize the before mentioned effects during the early phase of sepsis (Wang et al. 2001. Peptides 22: 1835-1840).

For other diseases blocking of ADM may be beneficial to a certain extent. However, it might also be detrimental if ADM is totally neutralized, as a certain amount of ADM may be required for several physiological functions. In many reports it was emphasized, that the administration of ADM may be beneficial in certain diseases. In contrast thereto, in other reports ADM was reported as being life threatening when administered in certain conditions.

WO2013/072510 describes a non-neutralizing anti-ADM antibody for use in therapy of a severe chronical or acute disease or acute condition of a patient for the reduction of the mortality risk for said patient.

WO2013/072511 describes a non-neutralizing anti-ADM antibody for use in therapy of a chronical or acute disease or acute condition of a patient for prevention or reduction of organ dysfunction or organ failure.

WO2013/072512 describes a non-neutralizing anti-ADM antibody that is an ADM stabilizing antibody that enhances the half-life (t_{1/2} half retention time) of adrenomedullin in serum, blood, plasma. This ADM stabilizing antibody blocks the bioactivity of ADM to less than 80%.

WO2013/072513 describes a non-neutralizing anti-ADM antibody for use in therapy of an acute disease or condition of a patient for stabilizing the circulation.

WO2013/072514 describes a non-neutralizing anti-ADM antibody for regulating the fluid balance in a patient having a chronic or acute disease or acute condition.

WO2017/182561 describes methods for determining the total amount or active DPP3 in a sample of a patient for the diagnosis of a disease related to necrotic processes. It further describes a method of treatment of necrosis-related diseases by antibodies directed to DPP3.

WO2021/170838 describes a method for therapy guidance and/or therapy mornitoring and/or therapy stratification in patients with shock and patients running into shock by determining the level of DPP3 and if the level is below a threshold of preferably 50 ng/ml, then administereing an anti-ADM antibody.

High DPP3 blood levels are associated with higher organ dysfunction scores, the need of cardiovascular support and the development of myocardial dysfunction, refractory shock, acute kidney injury and increased short-term mortality. Based on these clinical associations, it is plausible that active DPP3 released in the blood of shock patients is a factor contributing to the deterioration of vascular tone in shock syndromes by disabling the vasopressor effects of endogenous Ang II (Malovan G, Hierzberger B, Suraci S, Schaefer M, Santos K, Jha S, Macheroux P. FEBS J. 2022 Mar. 12. doi: 10.1111/febs.16429.). In patients with shock the presence of endothelial dysfunction is detected with the help of the biomarker bio-ADM, whereas cardiac dysfunction is detected by measuring DPP3. DPP3 above a threshold has been used as exclusion criteria for the application of medicaments addressing a different pathway than DPP3, e.g., anti-ADM antibodies, in particular the anti-ADM antibody Adrecizumab, which is directed against the N-terminus of ADM. Adrecizumab therapy showed a better efficacy if patients with DPP3 above 50 ng/ml are excluded from treatment. However, it was the surprising finding of the present invention that if DPP3 is well below the threshold of 50 ng/ml, preferably below a threshold of 40 ng/ml or in the range between 22 ng/ml and 40 ng/ml patients treated with the anti-ADM antibody Adrecizumab have a higher chance to benefit from this treatment.

In other words, it is the surprising finding of the present invention, that in patients with shock and patients running into shock, the level of DPP3 in a bodily fluid sample is to be used for the therapy guidance and/or therapy mornitoring and/or therapy stratification with an anti-ADM antibody and/or anti-ADM antibody fragment and/or anti-ADM non-Ig scaffold if the level of DPP3 in a bodily fluid sample is below a threshold of 40 ng/ml or in the range between 22 ng/ml and 40 ng/ml.

### DESCRIPTION OF THE INVENTION

Subject-matter of the present invention is an anti-adrenomedullin (ADM) antibody or anti-ADM antibody fragment or anti-ADM non-Ig scaffold for use in therapy or prevention of shock in a patient, wherein said patient is characterized by having a level of DPP3 in a sample of bodily fluid below a threshold, wherein saidthreshold of DPP3 is </=40 ng/ml or is in the range between 22 ng/ml and 40 ng/ml and said anti-ADM antibody or anti-ADM fragment or anti-ADM non-Ig scaffold binds to the N-terminal part (amino acid 1-21) of ADM: YRQSMNNFQGLRSFGCRFGTC (SEQ ID No. 14).

One embodiment of the present application relates to an anti-ADM antibody or anti-ADM antibody fragment or anti-ADM non-Ig scaffold for use in therapy or prevention of shock in a patient, wherein said shock is selected from the group comprising shock due to hypovolemia, cardiogenic shock, obstructive shock and distributive shock, in particular cardiogenic shock or septic shock.

Another embodiment of the present application relates to an anti-ADM antibody or anti-ADM antibody fragment or anti-ADM non-Ig scaffold for use in therapy or prevention of shock in a patient, wherein
in case of cardiogenic shock said patient may have suffered an acute coronary syndrome (e.g. acute myocardial infarction) or wherein said patient has heart failure (e.g. acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g. burns, heat stroke) or third-space loss in the setting of pancreatitis, cirrhosis, intestinal obstruction, trauma, or
in case of obstructive shock said patient may have suffered a cardiac tamponade, tension pneumothorax, pulmonary embolism or aortic stenosis, or
in case of distributive shock said patient may have septic shock, neurogenic shock, anaphylactic shock or shock due to adrenal crisis.

One preferred embodiment of the present application relates to an anti-ADM antibody or anti-ADM antibody fragment or anti-ADM non-Ig scaffold for use in therapy or prevention of shock in a patient, wherein said threshold of DPP3 in a sample of bodily fluid of said patient is ≤30 ng/ml or in the range between 22 and 30 ng/mL.

One preferred embodiment of the present application relates to an anti-ADM antibody or anti-ADM antibody fragment or anti-ADM non-Ig scaffold for use in therapy or prevention of shock in a patient, wherein said threshold of DPP3 in a sample of bodily fluid of said patient is ≤25 ng/ml or in the range between 22 and 25 ng/mL.

One specific embodiment of the present application relates to an anti-ADM antibody or anti-ADM antibody fragment or anti-ADM non-Ig scaffold for use in therapy or prevention of shock in a patient, wherein the level of DPP3 is determined by contacting said sample of bodily fluid with a capture binder that binds specifically to DPP3.

Another embodiment of the present application relates to an anti-ADM antibody or anti-ADM antibody fragment or anti-ADM non-Ig scaffold for use in therapy or prevention of shock in a patient, wherein either the level of DPP3 protein and/or the level of active DPP3 is determined and compared to a threshold.

One embodiment of the present application relates to an anti-ADM antibody or anti-ADM antibody fragment or anti-ADM non-Ig scaffold for use in therapy or prevention of shock in a patient, wherein said patient is additionally characterized by having a level of ADM-NH₂ above a threshold. The level of ADM —NH₂ is measured in order to identify patients in shock.

One preferred embodiment of the present application relates to an anti-ADM antibody or anti-ADM antibody fragment or anti-ADM non-Ig scaffold for use in therapy or prevention of shock in a patient, wherein said threshold of ADM-NH₂ in a sample of bodily fluid of said patient is between 40 and 100 pg/mL, more preferred between 50 and 90 pg/mL, even more preferred between 60 and 80 pg/mL, most preferred said threshold is 70 pg/mL.

Another embodiment of the present application relates to an anti-ADM antibody or anti-ADM antibody fragment or anti-ADM non-Ig scaffold for use in therapy or prevention of shock in a patient, wherein the level of ADM-NH₂ is determined by contacting said sample of bodily fluid with a capture binder that binds specifically to ADM-NH₂.

Another preferred embodiment of the present application relates to an anti-ADM antibody or anti-ADM antibody fragment or anti-ADM non-Ig scaffold for use in therapy or prevention of shock in a patient, wherein the sample of bodily fluid of said patient is selected from the group of blood, serum, plasma, urine, cerebrospinal fluid (CSF), and saliva.

Another specific embodiment of the present application relates to an anti-ADM antibody or anti-ADM antibody fragment or anti-ADM non-Ig scaffold for use in therapy or prevention of shock in a patient, wherein said anti-ADM antibody or anti-ADM antibody fragment or anti-ADM non-Ig scaffold recognizes and binds to the N-terminal end (amino acid 1) of ADM-Gly and/or ADM-NH₂.

A further embodiment of the present application relates to an anti-ADM antibody or anti-ADM antibody fragment or anti-ADM non-Ig scaffold for use in therapy or prevention of shock in a patient, wherein said antibody, antibody fragment or non-Ig scaffold does not bind to the C-terminal portion of ADM, having the sequence amino acid 43-52 of ADM: PRSKISPQGY-NH₂ (SEQ ID NO: 24).

One embodiment of the present application relates to an anti-ADM antibody or anti-ADM antibody fragment or anti-ADM non-Ig scaffold for use in therapy or prevention of shock in a patient, wherein said antibody or fragment or scaffold blocks the bioactivity of ADM not more than 80%, preferably not more than 50%.

Another embodiment of the present application relates to an anti-ADM antibody or anti-ADM antibody fragment or anti-ADM non-Ig scaffold for use in therapy or prevention of shock in a patient, wherein said antibody or fragment is a monoclonal antibody or fragment that binds to ADM or an antibody fragment thereof, wherein the heavy chain comprises the sequences:

CDR1:
SEQ ID NO: 1
GYTFSRYW
CDR2:
SEQ ID NO: 2
ILPGSGST
CDR3:
SEQ ID NO: 3
TEGYEYDGFDY

and wherein the light chain comprises the sequences:

CDR1:
SEQ ID NO: 4
QSIVYSNGNTY
CDR2:
RVS
CDR3:
SEQ ID NO: 5
FQGSHIPYT.

Another embodiment of the present application relates to an anti-ADM antibody or anti-ADM antibody fragment or anti-ADM non-Ig scaffold for use in therapy or prevention of shock in a patient, wherein said antibody or fragment comprises a sequence selected from the group comprising as a VH region:

(AM-VH-C)
SEQ ID NO: 6
QVQLQQSGAELMKPGASVKISCKATGYTFSRYWIEWVKQRPGHGLEWIGEILPGSGSTNYNE
KFKGKATITADTSSNTAYMQLSSLTSEDSAVYYCTEGYEYDGFDYWGQGTTLTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPK
(AM-VH1)
SEQ ID NO: 7
QVQLVQSGAEVKKPGSSVKVSCKASGYTFSRYWISWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH2-E40)
SEQ ID NO: 8
QVQLVQSGAEVKKPGSSVKVSCKASGYTFSRYWIEWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH3-T26-E55)
SEQ ID NO: 9
QVQLVQSGAEVKKPGSSVKVSCKATGYTFSRYWISWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH4-T26-E40-E55)
SEQ ID NO: 10
QVQLVQSGAEVKKPGSSVKVSCKATGYTFSRYWIEWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK

and comprises a sequence selected from the group comprising the following sequence as a VL region:

(AM-VL-C)
SEQ ID NO: 11
DVLLSQTPLSLPVSLGDQATISCRSSQSIVYSNGNTYLEWYLQKPGQSPKLLIYRVSNRFSGVP
DRFSGSGSGTDFTLKISRVEAEDLGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
(AM-VL1)
SEQ ID NO: 12
DVVMTQSPLSLPVTLGQPASISCRSSQSIVYSNGNTYLNWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
(AM-VL2-E40)
SEQ ID NO: 13
DVVMTQSPLSLPVTLGQPASISCRSSQSIVYSNGNTYLEWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC.

Another embodiment of the present application relates to an anti-ADM antibody or anti-ADM antibody fragment or anti-ADM non-Ig scaffold for use in therapy or prevention of shock in a patient, wherein said antibody or fragment comprises the following sequence as a heavy chain:

SEQ ID NO: 32
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNH
YTQKSLSLSPGK

or a sequence that is >95% identical to it,
and comprises the following sequence as a light chain:

SEQ ID NO: 33
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC

or a sequence that is >95% identical to it.

Subject-matter of the present application is also a pharmaceutical formulation for use in therapy or prevention of shock of a patient comprising an anti-ADM antibody or an anti-ADM antibody fragment or anti-ADM non-Ig scaffold.

One embodiment of the present application relates to a pharmaceutical formulation for use in therapy or prevention of shock of a patient, wherein said pharmaceutical formulation is a solution, preferably a ready-to-use solution.

Another embodiment of the present application relates to a pharmaceutical formulation for use in therapy or prevention of shock of a patient, wherein said pharmaceutical formulation is in a freeze-dried state.

One embodiment of the present application relates to a pharmaceutical formulation for use in therapy or prevention of shock of a patient, wherein said pharmaceutical formulation is administered intra-muscular.

One preferred embodiment of the present application relates to a pharmaceutical formulation for use in therapy or prevention of shock of a patient, wherein said pharmaceutical formulation is administered intra-vascular.

Another embodiment of the present application relates to a pharmaceutical formulation for use in therapy or prevention of shock of a patient, wherein said pharmaceutical formulation is administered via infusion.

Another specific embodiment of the present application relates to a pharmaceutical formulation for use in therapy or prevention of shock of a patient, wherein said pharmaceutical formulation is to be administered systemically.

Another embodiment of the present application relates to a method of therapy or prevention of shock in a patient, the method comprising administering an anti-adrenomedullin (ADM) antibody or an anti-adrenomedullin antibody fragment or anti-ADM non-Ig scaffold to said patient, the method further comprising the steps:
determining the level of DPP3 in a sample of bodily fluid of said subject,
comparing said level of determined DPP3 to a pre-determined threshold, and
   wherein said patient is treated if said determined level of DPP3 is below said pre-determined threshold,
   wherein said pre-determined threshold of DPP3 is </=40 ng/ml, or in the range between 22 ng/ml and 40 ng/ml, and
   wherein said anti-ADM antibody or anti-ADM fragment or anti-ADM non-Ig scaffold binds to the N-terminal part (aa 1-21) of ADM: YRQSMNNFQGLRSFGCRFGTC (SEQ ID No. 14).

Another specific embodiment of the present application relates to a method of therapy or prevention of shock in a patient, the method comprising administering an anti-adrenomedullin (ADM) antibody or an anti-adrenomedullin antibody fragment or anti-ADM non-Ig scaffold to said patient, wherein the method is additionally comprising the steps:
determining the level of ADM-NH₂ in a sample of bodily fluid of said subject,
comparing said level of ADM-NH₂ to a pre-determined threshold,
   wherein said patient is treated if said determined level of ADM-NH₂ is above said pre-determined threshold level.

Subject-matter of the present invention is an anti-adrenomedullin (ADM) antibody or anti-ADM antibody fragment or anti-ADM non-Ig scaffold for use in the treatment or prevention of shock in a patient, wherein said patient is characterized by having a level of DPP3 in a sample of bodily fluid below a threshold, wherein said threshold of DPP3 is </=40 ng/ml, or in the range between 22 ng/ml and 40 ng/ml and said anti-ADM antibody or anti-ADM fragment or anti-ADM non-Ig scaffold binds to the N-terminal part (amino acid 1-21) of ADM: YRQSMNNFQGLRSFGCRFGTC (SEQ ID No. 14).

One embodiment of the present application relates to an anti-ADM antibody or anti-ADM antibody fragment or anti-ADM non-Ig scaffold for use in the treatment or prevention of shock in a patient, wherein said shock is selected from the group comprising shock due to hypovolemia, cardiogenic shock, obstructive shock and distributive shock, in particular cardiogenic shock or septic shock.

Another embodiment of the present application relates to an anti-ADM antibody or anti-ADM antibody fragment or anti-ADM non-Ig scaffold for use in the treatment or prevention of shock in a patient, wherein
in case of cardiogenic shock said patient may have suffered an acute coronary syndrome (e.g. acute myocardial infarction) or wherein said patient has heart failure (e.g. acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g. burns, heat stroke) or third-space loss in the setting of pancreatitis, cirrhosis, intestinal obstruction, trauma, or
in case of obstructive shock said patient may have suffered a cardiac tamponade, tension pneumothorax, pulmonary embolism or aortic stenosis, or
in case of distributive shock said patient may have septic shock, neurogenic shock, anaphylactic shock or shock due to adrenal crisis.

One preferred embodiment of the present application relates to an anti-ADM antibody or anti-ADM antibody fragment or anti-ADM non-Ig scaffold for use in the treatment or prevention of shock in a patient, wherein said threshold of DPP3 in a sample of bodily fluid of said patient is ≤30 ng/ml or in the range between 22 and 30 ng/mL.

One preferred embodiment of the present application relates to an anti-ADM antibody or anti-ADM antibody fragment or anti-ADM non-Ig scaffold for use in the treatment or prevention of shock in a patient, wherein said threshold of DPP3 in a sample of bodily fluid of said patient is ≤25 ng/ml or in the range between 22 and 25 ng/mL.

One specific embodiment of the present application relates to an anti-ADM antibody or anti-ADM antibody fragment or anti-ADM non-Ig scaffold for use in the treatment or prevention of shock in a patient, wherein the level of DPP3 is determined by contacting said sample of bodily fluid with a capture binder that binds specifically to DPP3.

Another embodiment of the present application relates to an anti-ADM antibody or anti-ADM antibody fragment or anti-ADM non-Ig scaffold for use in the treatment or prevention of shock in a patient, wherein either the level of DPP3 protein and/or the level of active DPP3 is determined and compared to a predetermined threshold.

One embodiment of the present application relates to an anti-ADM antibody or anti-ADM antibody fragment or anti-ADM non-Ig scaffold for use in the treatment or prevention of shock in a patient, wherein said patient is additionally characterized by having a level of ADM-NH₂ above a threshold.

One preferred embodiment of the present application relates to an anti-ADM antibody or anti-ADM antibody fragment or anti-ADM non-Ig scaffold for use in the treatment or prevention of shock in a patient, wherein said threshold of ADM-NH₂ in a sample of bodily fluid of said patient is between 40 and 100 pg/mL, more preferred between 50 and 90 pg/mL, even more preferred between 60 and 80 pg/mL, most preferred said threshold is 70 pg/mL.

Another embodiment of the present application relates to an anti-ADM antibody or anti-ADM antibody fragment or anti-ADM non-Ig scaffold for use in the treatment or prevention of shock in a patient, wherein the level of ADM-NH₂ is determined by contacting said sample of bodily fluid with a capture binder that binds specifically to ADM-NH₂.

Another preferred embodiment of the present application relates to an anti-ADM antibody or anti-ADM antibody fragment or anti-ADM non-Ig scaffold for use in the treatment or prevention of shock in a patient, wherein the sample of bodily fluid of said patient is selected from the group of blood, serum, plasma, urine, cerebrospinal fluid (CSF), and saliva.

In a preferred embodiment, the bio-ADM is measured from plasma. It is however typical in the technical lifecycle improvement of measurement of analytes that possibilities exist to measure such analytes in other—at least blood-based—matrices, not only plasma. For instance, in case of bio-ADM, another technology has been developed, which uses whole (EDTA−) blood called IB10 Sphingotest® bio-ADM (https://www.nexus-dx.com/wp-content/uploads/2020/07/bio-ADM-IFU-REV-A.pdfq. The IB10 Sphingotest® bio-ADM® is a rapid point-of-care (POC) immunoassay for the in vitro quantitative determination of human amidated adrenomedullin peptide (1-52), in the following referred to as bioactive adrenomedullin (bio-ADM), in human EDTA whole blood and plasma.

Another specific embodiment of the present application relates to an anti-ADM antibody or anti-ADM antibody fragment or anti-ADM non-Ig scaffold for use in the treatment or prevention of shock in a patient, wherein said anti-ADM antibody or anti-ADM antibody fragment or anti-ADM non-Ig scaffold recognizes and binds to the N-terminal end (amino acid 1) of ADM-Gly and/or ADM-NH₂.

A further embodiment of the present application relates to an anti-ADM antibody or anti-ADM antibody fragment or anti-ADM non-Ig scaffold for use in the treatment or prevention of shock in a patient, wherein said antibody, antibody fragment or non-Ig scaffold does not bind to the C-terminal portion of ADM, having the sequence amino acid 43-52 of ADM: PRSKISPQGY-NH₂ (SEQ ID NO: 24).

One embodiment of the present application relates to an anti-ADM antibody or anti-ADM antibody fragment or anti-ADM non-Ig scaffold for use in the treatment or prevention of shock in a patient, wherein said antibody or fragment or scaffold blocks the bioactivity of ADM not more than 80%, preferably not more than 50%.

Another embodiment of the present application relates to an anti-ADM antibody or anti-ADM antibody fragment or anti-ADM non-Ig scaffold for use in the treatment or prevention of shock in a patient, wherein said antibody or fragment is a monoclonal antibody or fragment that binds to ADM or an antibody fragment thereof, wherein the heavy chain comprises the sequences:

CDR1:
SEQ ID NO: 1
GYTFSRYW
CDR2:
SEQ ID NO: 2
ILPGSGST
CDR3:
SEQ ID NO: 3
TEGYEYDGFDY

and wherein the light chain comprises the sequences:

CDR1:
SEQ ID NO: 4
QSIVYSNGNTY
CDR2:
RVS
CDR3:
SEQ ID NO: 5
FQGSHIPYT.

Another embodiment of the present application relates to an anti-ADM antibody or anti-ADM antibody fragment or anti-ADM non-Ig scaffold for use in the treatment or prevention of shock in a patient, wherein said antibody or fragment comprises a sequence selected from the group comprising as a VH region:

(AM-VH-C)
SEQ ID NO: 6
QVQLQQSGAELMKPGASVKISCKATGYTFSRYWIEWVKQRPGHGLEWIGEILPGSGSTNYNE
KFKGKATITADTSSNTAYMQLSSLTSEDSAVYYCTEGYEYDGFDYWGQGTTLTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPK
(AM-VH1)
SEQ ID NO: 7
QVQLVQSGAEVKKPGSSVKVSCKASGYTFSRYWISWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH2-E40)
SEQ ID NO: 8
QVQLVQSGAEVKKPGSSVKVSCKASGYTFSRYWIEWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH3-T26-E55)
SEQ ID NO: 9
QVQLVQSGAEVKKPGSSVKVSCKATGYTFSRYWISWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
(AM-VH4-T26-E40-E55)
SEQ ID NO: 10
QVQLVQSGAEVKKPGSSVKVSCKATGYTFSRYWIEWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK

and comprises a sequence selected from the group comprising the following sequence as a VL region:

(AM-VL-C)
SEQ ID NO: 11
DVLLSQTPLSLPVSLGDQATISCRSSQSIVYSNGNTYLEWYLQKPGQSPKLLIYRVSNRFSGVP
DRFSGSGSGTDFTLKISRVEAEDLGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
(AM-VL1)
SEQ ID NO: 12
DVVMTQSPLSLPVTLGQPASISCRSSQSIVYSNGNTYLNWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
(AM-VL2-E40)
SEQ ID NO: 13
DVVMTQSPLSLPVTLGQPASISCRSSQSIVYSNGNTYLEWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC.

Another embodiment of the present application relates to an anti-ADM antibody or anti-ADM antibody fragment or anti-ADM non-Ig scaffold for use in the treatment or prevention of shock in a patient, wherein said antibody or fragment comprises the following sequence as a heavy chain:

SEQ ID NO: 32
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNH
YTQKSLSLSPGK

or a sequence that is >95% identical to it,
and comprises the following sequence as a light chain:

SEQ ID NO: 33
DVVLTQSPLSLPVTLGQPASISCRSSQSIVYSNGNTYLEWYLQRPGQSP
RLLIYRVSNRFSGVPDRFSGSGSGTDFTLKISRVEAEDVGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC

or a sequence that is >95% identical to it.

Another embodiment of the present application relates to an anti-adrenomedullin (ADM) antibody or anti-ADM antibody fragment or anti-ADM non-Ig scaffold for use in therapy or prevention of shock in a patient, wherein the anti-adrenomedullin (ADM) antibody or anti-ADM antibody fragment or anti-ADM non-Ig scaffold binds to the N-terminal part (amino acid 1-10) of ADM: YRQSMNNFQG (SEQ ID No. 26).

Another embodiment of the present application relates to an anti-adrenomedullin (ADM) antibody or an anti-adrenomedullin antibody fragment or anti-ADM non-Ig scaffold for use in therapy, wherein said antibody or fragment or scaffold exhibits a binding affinity to ADM of at least 10′ M by label-free surface plasmon resonance using a Biacore 2000 system.

Another embodiment of the present application relates to an anti-adrenomedullin (ADM) antibody or anti-ADM antibody fragment or anti-ADM non-Ig scaffold for use in therapy or prevention of shock in a patient, wherein the anti-ADM antibody or anti-ADM antibody fragment or anti-ADM non-Ig scaffold exhibits an affinity towards human ADM is between 1×10⁻⁹ to 3×10⁻⁹ M by label-free surface plasmon resonance using a Biacore 2000 system.

Another embodiment of the present application relates to an anti-adrenomedullin (ADM) antibody or anti-ADM antibody fragment or anti-ADM non-Ig scaffold for use in therapy or prevention of shock in a patient, wherein the anti-ADM antibody or anti-ADM antibody fragment or anti-ADM non-Ig scaffold is an IgG1 antibody.

Subject-matter of the present application is also a pharmaceutical formulation for use in the treatment or prevention of shock of a patient comprising an anti-ADM antibody or an anti-ADM antibody fragment or anti-ADM non-Ig scaffold.

One embodiment of the present application relates to a pharmaceutical formulation for use in the treatment or prevention of shock of a patient, wherein said pharmaceutical formulation is a solution, preferably a ready-to-use solution.

Another embodiment of the present application relates to a pharmaceutical formulation for use in the treatment or prevention of shock of a patient, wherein said pharmaceutical formulation is in a freeze-dried state.

One embodiment of the present application relates to a pharmaceutical formulation for use in the treatment or prevention of shock of a patient, wherein said pharmaceutical formulation is administered intra-muscular.

One preferred embodiment of the present application relates to a pharmaceutical formulation for use in the treatment or prevention of shock of a patient, wherein said pharmaceutical formulation is administered intra-vascular.

Another embodiment of the present application relates to a pharmaceutical formulation for use in the treatment or prevention of shock of a patient, wherein said pharmaceutical formulation is administered via infusion.

Another specific embodiment of the present application relates to a pharmaceutical formulation for use in the treatment or prevention of shock of a patient, wherein said pharmaceutical formulation is to be administered systemically.

Another embodiment of the present application relates to a method of treatment or prevention of shock in a patient, the method comprising administering an anti-adrenomedullin (ADM) antibody or an anti-adrenomedullin antibody fragment or anti-ADM non-Ig scaffold to said patient, the method further comprising the steps:
determining the level of DPP3 in a sample of bodily fluid of said subject,
comparing said level of determined DPP3 to a pre-determined threshold, and
   wherein said patient is treated if said determined level of DPP3 is below said pre-determined threshold,
   wherein said pre-determined threshold of DPP3 is </=40 ng/ml, or in the range between 22 ng/ml and 40 ng/ml, and
   wherein said anti-ADM antibody or anti-ADM fragment or anti-ADM non-Ig scaffold binds to the N-terminal part (aa 1-21) of ADM: YRQSMNNFQGLRSFGCRFGTC (SEQ ID No. 14).

Another specific embodiment of the present application relates to a method of treatment or prevention of shock in a patient, the method comprising administering an anti-adrenomedullin (ADM) antibody or an anti-adrenomedullin antibody fragment or anti-ADM non-Ig scaffold to said patient, wherein the method is additionally comprising the steps:
determining the level of ADM-NH₂ in a sample of bodily fluid of said subject,
comparing said level of ADM-NH₂ to a pre-determined threshold,
   wherein said patient is treated if said determined level of ADM-NH₂ is above said pre-determined threshold level.

In one embodiment of the invention either the level of DPP3 protein and/or the level of active DPP3 is determined and compared to a threshold level.

In a specific embodiment of the invention a threshold of DPP3 in a sample of bodily fluid of said patient is ≤30 ng/ml or in the range between 22 and 30 ng/mL.

In another specific embodiment of the invention a threshold of DPP3 in a sample of bodily fluid of said patient is ≤25 ng/ml or in the range between 22 and 25 ng/mL.

The level of DPP3 as the amount of DPP3 protein and/or DPP3 activity in a sample of bodily fluid of said subject may be determined by different methods, e.g., immunoassays, activity assays, mass spectrometric methods etc.

According to the present invention, any types of binding assays (immunoassays and analogous assays, which use other types of antigen-specific binders instead of antibodies), and b) DPP3 enzyme activity assays, which are specific for DPP3 by specifically capturing DPP3 from a sample using a specific binder (anti-DPP3 antibody or other type of binder) prior to determination of enzyme activity) may be used to determine the level of DPP3 ins said sample.

DPP3 activity can be measured by detection of cleavage products of DPP3 specific substrates. Known peptide hormone substrates include Leu-enkephalin, Met-enkephalin, endomorphin 1 and 2, valorphin, β-casomorphin, dynorphin, proctolin, ACTH (Adrenocorticotropic hormone) and MSH (melanocyte-stimulating hormone; Abramić et al. 2000, Baršun et al. 2007, Dhanda et al. 2008). The cleavage of mentioned peptide hormones as well as other untagged oligopeptides (e.g. Ala-Ala-Ala-Ala, Dhanda et al. 2008) can be monitored by detection of the respective cleavage products. Detection methods include, but are not limited to, HPLC analysis (e.g. Lee & Snyder 1982), mass spectrometry (e.g. Abramić et al. 2000), H1-NMR analysis (e.g. Vandenberg et al. 1985), capillary zone electrophoresis (CE; e.g. Baršun et al. 2007), thin layer chromatography (e.g. Dhanda et al. 2008) or reversed phase chromatography (e.g. Mazocco et al. 2006).

Detection of fluorescence due to hydrolysis of fluorogenic substrates by DPP3 is a standard procedure to monitor DPP3 activity. Those substrates are specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) coupled to a fluorophore. Fluorophores include but are not limited to β-naphtylamide (2-naphtylamide, βNA, 2NA), 4-methoxy-β-naphtylamide (4-methoxy-2-naphtylamide) and 7-amido-4-methylcoumarin (AMC, MCA; Abramić et al. 2000, Ohkubo et al. 1999). Cleavage of these fluorogenic substrates leads to the release of fluorescent β-naphtylamine or 7-amino-4-methylcoumarin respectively. In a liquid phase assay or an ECA substrate and DPP3 are incubated in for example a 96 well plate format and fluorescence is measured using a fluorescence detector (Ellis & Nuenke 1967). Additionally, DPP3 carrying samples can be immobilized and divided on a gel by electrophoresis, gels stained with fluorogenic substrate (e.g. Arg-Arg-βNA) and Fast Garnet GBC and fluorescent protein bands detected by a fluorescence reader (Ohkubo et al. 1999). The same peptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) can be coupled to chromophores, such as p-nitroanilide diacetate. Detection of color change due to hydrolysis of chromogenic substrates can be used to monitor DPP3 activity.

Another option for the detection of DPP3 activity is a Protease-Glo™ Assay (commercially available at Promega). In this embodiment of said method DPP3 specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) are coupled to aminoluciferin. Upon cleavage by DPP3, aminoluciferin is released and serves as a substrate for a coupled luciferase reaction that emits detectable luminescence.

In a preferred embodiment DPP3 activity is measured by addition of the fluorogenic substrate Arg-Arg-βNA and monitoring fluorescence in real time.

In a specific embodiment of said method for determining active DPP3 in a bodily fluid sample of a subject said capture binder reactive with DPP3 is immobilized on a solid phase.

The test sample is passed over the immobile binder, and DPP3, if present, binds to the binder and is itself immobilized for detection. A substrate may then be added, and the reaction product may be detected to indicate the presence or amount of DPP3 in the test sample. For the purposes of the present description, the term “solid phase” may be used to include any material or vessel in which or on which the assay may be performed and includes, but is not limited to: porous materials, nonporous materials, test tubes, wells, slides, agarose resins (e.g. Sepharose from GE Healthcare Life Sciences), magnetic particals (e.g. Dynabeads™ or Pierce™ magnetic beads from Thermo Fisher Scientific), etc.

In another embodiment of the invention, the level of DPP3 is determined by contacting said sample of bodily fluid with a capture binder that binds specifically to DPP3.

In another preferred embodiment of the invention, said capture binder for determining the level of DPP3 may be selected from the group of antibody, antibody fragment or non-IgG scaffold.

In a specific embodiment of the invention, said capture binder is an antibody.

The amount of DPP3 protein and/or DPP3 activity in a sample of bodily fluid of said subject may be determined for example by one of the following methods:
1. Luminescence immunoassay for the quantification of DPP3 protein concentrations (LIA) (Rehfeld et al., 2019 JALM 3(6): 943-953).

The LIA is a one-step chemiluminescence sandwich immunoassay that uses white high-binding polystyrene microtiter plates as solid phase. These plates are coated with monoclonal anti-DPP3 antibody AK2555 (capture antibody). The tracer anti-DPP3 antibody AK2553 is labeled with MA70-acridinium-NHS-ester and used at a concentration of 20 ng per well. Twenty microliters of samples (e.g. serum, heparin-plasma, citrate-plasma or EDTA-plasma derived from patients' blood) and calibrators are pipetted into coated white microtiter plates. After adding the tracer antibody AK2553, the microtiter plates are incubated for 3 h at room temperature and 600 rpm. Unbound tracer is then removed by 4 washing steps (350 μL per well). Remaining chemiluminescence is measured for is per well by using a microtiter plate luminometer. The concentration of DPP3 is determined with a 6-point calibration curve. Calibrators and samples are preferably run in duplicate.
2. Enzyme capture activity assay for the quantification of DPP3 activity (ECA) (Rehfeld et al., 2019 JALM 3(6): 943-953).

The ECA is a DPP3-specific activity assay that uses black high-binding polystyrene microtiter plates as solid phase. These plates are coated with monoclonal anti-DPP3 antibody AK2555 (capture antibody). Twenty microliters of samples (e.g. serum, heparin-plasma, citrate-plasma, EDTA-plasma, cerebrospinal fluid and urine) and calibrators are pipetted into coated black microtiter plates. After adding assay buffer (200 μL), the microtiter plates are incubated for 2 h at 22° C. and 600 rpm. DPP3 present in the samples is immobilized by binding to the capture antibody. Unbound sample components are removed by 4 washing steps (350 μL per well). The specific activity of immobilized DPP3 is measured by the addition of the fluorogenic substrate, Arg-Arg-β-Naphthylamide (Arg2-βNA), in reaction buffer followed by incubation at 37° C. for 1 h. DPP3 specifically cleaves Arg2-βNA into Arg-Arg dipeptide and fluorescent β-naphthylamine. Fluorescence is measured with a fluorometer using an excitation wavelength of 340 nm and emission is detected at 410 nm. The activity of DPP3 is determined with a 6-point calibration curve. Calibrators and samples are preferably run in duplicates.
3. Liquid-phase assay for the quantification of DPP3 activity (LAA) (modified from Jones et al., Analytical Biochemistry, 1982).

The LAA is a liquid phase assay that uses black non-binding polystyrene microtiter plates to measure DPP3 activity. Twenty microliter of samples (e.g. serum, heparin-plasma, citrate-plasma) and calibrators are pipetted into non-binding black microtiter plates. After addition of fluorogenic substrate, Arg2-βNA, in assay buffer (200 μL), the initial PNA fluorescence (T=0) is measured in a fluorimeter using an excitation wavelength of 340 nm and emission is detected at 410 nm. The plate is then incubated at 37° C. for 1 hour. The final fluorescence of (T=60) is measured. The difference between final and initial fluorescence is calculated. The activity of DPP3 is determined with a 6-point calibration curve. Calibrators and samples are preferably run in duplicates.

In a specific embodiment an assay is used for determining the level of DPP3, wherein the assay sensitivity of said assay is able to quantify the DPP3 of healthy subjects and is <20 ng/ml, preferably <30 ng/ml and more preferably <40 ng/ml.
4. Another immunoassay method for measuring DPP3 from a plasma of whole blood sample is available, IB10 Sphingotest® DPP3 (https://www.nexus-dx.com/wp-content/uploads/2020/11/DPP3-022-00072-IFU-REV-B_8x11.pdf).

The IB10 Sphingotest® DPP3 is a rapid point-of-care (POC) immunoassay for the in vitro quantitative determination of Dipeptidyl Peptidase 3 (DPP3) in human EDTA whole blood and plasma. The Nexus IB10 immunochemistry system combines chemistry with microfluidics and centrifugal flow to rapidly prepare a cell free plasma from whole blood that can then be moved through a channel to rehydrate, solubilize and mix with freeze dried immunoconjugates

In a specific embodiment, said binder exhibits a binding affinity to DPP3 of at least 10⁷ M⁻¹, preferred 10⁸ M⁻¹, more preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

In another embodiment of the invention, said sample of bodily fluid is selected from the group of whole blood, plasma, and serum.

Mature ADM, bio-ADM and ADM-NH₂ is used synonymously throughout this application and is a molecule according to SEQ ID No.: 20.

A bodily fluid according to the present invention is in one particular embodiment a blood sample. A blood sample may be selected from the group comprising whole blood, serum and plasma. In a specific embodiment of the method said sample is selected from the group comprising human citrate plasma, heparin plasma and EDTA plasma.

In a specific embodiment an assay is used for determining the level ADM-NH₂, wherein the assay sensitivity of said assay is able to quantify the mature ADM-NH₂ of healthy subjects and is <70 pg/ml, preferably <40 pg/ml and more preferably <10 pg/ml.

In a specific embodiment of the invention the threshold for ADM-NH₂ is between 40 and 100 pg/mL, more preferred between 50 and 90 pg/mL, even more preferred between 60 and 80, most preferred a threshold of 70 pg/ml is applied.

In a specific embodiment of the invention a threshold for plasma ADM-NH₂ is the 5 fold median concentration, preferably the 4 fold median concentration, more preferred the 3 fold median concentration, most preferred the 2 fold median concentration of a normal healthy population.

In a specific embodiment, said binder exhibits a binding affinity to ADM-NH₂ of at least 10⁷ M⁻¹, preferred 10⁸ M⁻¹, preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

To determine the affinity of the antibodies to Adrenomedullin, the kinetics of binding of Adrenomedullin to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare), (Lorenz et al. 2011. Antimicrob Agents Chemother. 55 (1): 165-173).

In a specific embodiment, said binder is selected from the group comprising an antibody or an antibody fragment or a non-Ig scaffold binding to ADM-NH₂.

In a specific embodiment an assay is used for determining the level of ADM-NH₂, wherein such assay is a sandwich assay, preferably a fully automated assay.

In one embodiment such assay for determining the level of the biomarkers (DPP3 and/or ADM-NH₂) is a sandwich immunoassay using any kind of detection technology including but not restricted to enzyme label, chemiluminescence label, electrochemiluminescence label, preferably a fully automated assay. In one embodiment of the diagnostic method such an assay is an enzyme labeled sandwich assay.

Examples of automated or fully automated assay comprise assays that may be used for one of the following systems: Roche Elecsys®, Abbott Architect®, Siemens Centauer®, Brahms Kryptor®, BiomerieuxVidas®, Alere Triage®.

A variety of immunoassays are known and may be used for the assays and methods of the present invention, these include: radioimmunoassays (“RIA”), homogeneous enzyme-multiplied immunoassays (“EMIT”), enzyme linked immunoadsorbent assays (“ELISA”), apoenzyme reactivation immunoassay (“ARIS”), dipstick immunoassays and immuno-chromatography assays.

In one embodiment of the invention such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, Biomerieux Vidas®, Alere Triage®.

In one embodiment of the invention it may be a so-called POC-test (point-of-care) that is a test technology, which allows performing the test within less than 1 hour near the patient without the requirement of a fully automated assay system. One example for this technology is the immunochromatographic test technology.

In a preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer, Encyclopedia of chemical technology, 4th ed., executive editor, J. I. Kroschwitz; editor, M. Howe-Grant, John Wiley & Sons, 1993, vol. 15, p. 518-562, incorporated herein by reference, including citations on pages 551-562). Preferred chemiluminescent dyes are acridiniumesters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In a specific embodiment at least one of said two binders is labeled in order to be detected.

The ADM-NH₂ levels of the present invention have been determined with the described ADM-NH₂ assay (Weber et al. 2017. JALM 2(2):1-4). The DPP3 levels of the present invention have been determined with the described DPP3-assays as outlined in the examples (Rehfeld et al. 2019. JALM 3(6): 943-953). The mentioned threshold values above might be different in other assays, if these have been calibrated differently from the assay systems used in the present invention. Therefore, the mentioned cut-off values above shall apply for such differently calibrated assays accordingly, taking into account the differences in calibration. One possibility of quantifying the difference in calibration is a method comparison analysis (correlation) of the assay in question with the respective biomarker assay used in the present invention by measuring the respective biomarker (e.g. bio-ADM, DPP3) in samples using both methods. Another possibility is to determine with the assay in question, given this test has sufficient analytical sensitivity, the median biomarker level of a representative normal population, compare results with the median biomarker levels as described in the literature and recalculate the calibration based on the difference obtained by this comparison. With the calibration used in the present invention, samples from normal (healthy) subjects have been measured: median plasma bio-ADM (mature ADM-NH₂) was 24.7 pg/ml, the lowest value 11 pg/ml and the 99^{th} percentile 43 pg/ml (Marino et al. 2014. Critical Care 18:R34). With the calibration used in the present invention, samples from 5,400 normal (healthy) subjects (swedish single-center prospective population-based Study (MPP-RES)) have been measured: median (interquartile range) plasma DPP3 was 14.5 ng/ml (11.3 ng/ml-19 ng/ml). It was shown that DPP3 concentrations strongly correlate with DPP3 activity in the blood (Rehfeld et al. 2019. J Appl Lab Med 3: 943-953; Deniau et al. 2019. Eur J Heart Fail 22: 290-299). Consequently, the level of active DPP3 may be determined using respective thresholds and threshold ranges that correspond to the threshold and threshold ranges used by determining the level of DPP3 protein.

In the present invention, the level of ADM —NH₂ is therefore measured in order to identify patients having an increased risk of running into shock.

In another specific embodiment of the invention, said shock is selected from the group comprising shock due to hypovolemia, cardiogenic shock, obstructive shock and distributive shock, in particular cardiogenic or septic shock.

In a specific embodiment of the invention, said shock is selected from the group comprising:
in case of cardiogenic shock said patient has suffered an acute coronary syndrome (e.g. acute myocardial infarction) or has heart failure (e.g. acute decompensated heart failure), myocarditis, arrhythmia, cardiomyopathy, valvular heart disease, aortic dissection with acute aortic stenosis, traumatic chordal rupture or massive pulmonary embolism, or
in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g. burns, heat stroke) or third-space loss in the setting of pancreatitis, cirrhosis, intestinal obstruction, trauma, or
in case of obstructive shock said patient may have suffered a cardiac tamponade, tension pneumothorax, pulmonary embolism or aortic stenosis, or
in case of distributive shock said patient has septic shock, neurogenic shock, anaphylactic shock or shock due to adrenal crisis.

Shock is characterized by decreased oxygen delivery and/or increased oxygen consumption or inadequate oxygen utilization leading to cellular and tissue hypoxia. It is a life-threatening condition of circulatory failure and most commonly manifested as hypotension (systolic blood pressure less than 90 mm Hg or MAP less than 65 mmHg). Shock is divided into four main types based on the underlying cause: hypovolemic, cardiogenic, obstructive, and distributive shock (Vincent and De Backer 2014. N. Engl. J. Med. 370(6): 583).

Hypovolemic shock is characterized by decreased intravascular volume and can be divided into two broad subtypes: hemorrhagic and non-hemorrhagic. Common causes of hemorrhagic hypovolemic shock include gastrointestinal bleed, trauma, vascular etiologies (e.g. ruptured abdominal aortic aneurysm, tumor eroding into a major blood vessel) and spontaneous bleeding in the setting of anticoagulant use. Common causes of non-hemorrhagic hypovolemic shock include vomiting, diarrhea, renal loss, skin losses/insensible losses (e.g. burns, heat stroke) or third-space loss in the setting of pancreatitis, cirrhosis, intestinal obstruction, trauma. For review see Koya and Paul 2018. Shock. StatPearls [Internet]. Treasure Island (FL): StatPearls Publishing; 2019-2018 Oct. 27.

Cardiogenic shock (CS) is defined as a state of critical endorgan hypoperfusion due to reduced cardiac output. Notably, CS forms a spectrum that ranges from mild hypoperfusion to profound shock. Established criteria for the diagnosis of CS are: (i) systolic blood pressure, ≤90 mmHg for ≥30 min or vasopressors required to achieve a blood pressure ≥90 mmHg; (ii) pulmonary congestion or elevated left-ventricular filling pressures; (iii) signs of impaired organ perfusion with at least one of the following criteria: (a) altered mental status; (b) cold, clammy skin; (c) oliguria (<0.5 mL/kg/h or <30 mL/h); (d) increased serum-lactate (Reynolds and Hochman 2008. Circulation 117: 686-697). Acute myocardial infarction (AMI) with subsequent ventricular dysfunction is the most frequent cause of CS accounting for approximately 80% of cases. Mechanical complications such as ventricular septal (4%) or free wall rupture (2%), and acute severe mitral regurgitation (7%) are less frequent causes of CS after AMI. (Hochman et al. 2000. J Am Coll Cardiol 36: 1063-1070). Non-AMI-related CS may be caused by decompensated valvular heart disease, acute myocarditis, arrhythmias, etc. with heterogeneous treatment options. This translates in 40 000 to 50 000 patients per year in the USA and 60 000 to 70 000 in Europe. Despite advances in treatment mainly by early revascularization with subsequent mortality reduction, CS remains the leading cause of death in AMI with mortality rates still approaching 40-50% according to recent registries and randomized trials (Goldberg et al. 2009. Circulation 119: 1211-1219). Obstructive shock is due to a physical obstruction of the great vessels or the heart itself. Several conditions can result in this form of shock (e.g. cardiac tamponade, tension pneumothorax, pulmonary embolism, aortic stenosis). For review see Koya and Paul 2018. Shock. StatPearls [Internet]. Treasure Island (FL): StatPearls Publishing; 2019-2018 Oct. 27.

According to the cause, there are four types of distributive shock: neurogenic shock (decreased sympathetic stimulation leading to decreased vasal tone), anaphylactic shock, septic shock and shock due to adrenal crisis. In addition to sepsis, distributive shock can be caused by systemic inflammatory response syndrome (SIRS) due to conditions other than infection such as pancreatitis, burns or trauma. Other causes include, toxic shock syndrome (TSS), anaphylaxis (a sudden, severe allergic reaction), adrenal insufficiency (acute worsening of chronic adrenal insufficiency, destruction or removal of the adrenal glands, suppression of adrenal gland function due to exogenous steroids, hypopituitarism and metabolic failure of hormone production), reactions to drugs or toxins, heavy metal poisoning, hepatic (liver) insufficiency and damage to the central nervous system. For review see Koya and Paul 2018. Shock. StatPearls [Internet]. Treasure Island (FL): StatPearls Publishing; 2019-2018 Oct. 27.

Refractory shock has been defined as requirement of noradrenaline infusion of >0.5 g/kg/min despite adequate volume resuscitation. Mortality in these patients may be as high as 94% and the assessment and management of these patients requires a much more aggressive approach for survival. The term “refractory shock” is used when the tissue perfusion cannot be restored with the initial corrective measures employed (e.g. vasopressors) and may therefore be referred to as “high vasopressor-dependent” or “vasopressor-resistant” shock (Udupa and Shetty 2018. Indian J Respir Care 7: 67-72). Patients with refractory shock may have features of inadequate perfusion such as hypotension (mean arterial blood pressure <65 mmHg), tachycardia, cold peripheries, prolonged capillary refill time, and tachypnea consequent to the hypoxia and acidosis. Fever may be seen in septic shock. Other signs of hypoperfusion such as altered sensorium, hyperlactatemia, and oliguria may also be seen. These well-known signs of shock are not helpful in identifying whether the problem is at the pump (heart) or circuitry (vessels and tissues). Different types of shock can coexist, and all forms of shock can become refractory, as evidenced by unresponsiveness to high-dose vasopressors (Udupa and Shetty 2018. Indian J Respir Care 7: 67-72).

Septic shock is a potentially fatal medical condition that occurs when sepsis, which is organ injury or damage in response to infection, leads to dangerously low blood pressure and abnormalities in cellular metabolism. The Third International Consensus Definitions for Sepsis and Septic Shock (Sepsis-3) defines septic shock as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia. This combination is associated with hospital mortality rates greater than 40% (Singer et al. 2016. JAMA. 315 (8): 801-10). The primary infection is most commonly caused by bacteria, but also may be by fungi, viruses or parasites. It may be located in any part of the body, but most commonly in the lungs, brain, urinary tract, skin or abdominal organs. It can cause multiple organ dysfunction syndrome (formerly known as multiple organ failure) and death. Frequently, people with septic shock are cared for in intensive care units. It most commonly affects children, immunocompromised individuals, and the elderly, as their immune systems cannot deal with infection as effectively as those of healthy adults. The mortality rate from septic shock is approximately 25-50%.

The term “prevention” or any grammatical variation thereof (e.g., prevent, preventing, and prevention etc.), as used herein, includes but is not limited to, delaying the onset of symptoms, preventing relapse to a disease, increasing latency between symptomatic episodes, or a combination thereof. Prevention, as used herein, does not require the complete absence of symptoms.

The efficacy of non-neutralizing antibody targeted against the N-terminus of ADM was investigated in a survival study in CLP-induced sepsis in mice. Pre-treatment with the non-neutralizing antibody resulted in decreased catecholamine infusion rates, kidney dysfunction, and ultimately improved survival (Struck et al. 2013. Intensive Care Med Exp 1(1):22; Wagner et al. 2013. Intensive Care Med Exp 1(1):21).

Due to these positive results, a humanized version of an N-terminal anti-ADM antibody, named Adrecizumab, has been developed for further clinical development. Beneficial effects of Adrecizumab on vascular barrier function and survival were recently demonstrated in preclinical models of systemic inflammation and sepsis (Geven et al. 2018. Shock 50(6):648-654). In this study, pre-treatment with Adrecizumab attenuated renal vascular leakage in endotoxemic rats as well as in mice with CLP-induced sepsis, which coincided with increased renal expression of the protective peptide Ang-1 and reduced expression of the detrimental peptide vascular endothelial growth factor. Also, pre-treatment with Adrecizumab improved 7-day survival in CLP-induced sepsis in mice from 10 to 50% for single and from 0 to 40% for repeated dose administration. Moreover, in a phase I study, excellent safety and tolerability was demonstrated (see Example 6): no serious adverse events were observed, no signal of adverse events occurring more frequently in Adrecizumab-treated subjects was detected and no relevant changes in other safety parameters were found (Geven et al. 2017. Intensive Care Med Exp 5 (Suppl 2): 0427). Of particular interest is the proposed mechanism of action of Adrecizumab. Both animal and human data reveal a potent, dose-dependent increase of circulating ADM following administration of this antibody. Based on pharmacokinetic data and the lack of an increase in MR-proADM (an inactive peptide fragment derived from the same prohormone as ADM), the higher circulating ADM levels cannot be explained by an increased production.

A mechanistic explanation for this increase could be that the excess of antibody in the circulation may drain ADM from the interstitium to the circulation, since ADM is small enough to cross the endothelial barrier, whereas the antibody is not (Geven et al. 2018. Shock. 50(2):132-140 and Voors et al (J. Eur J Heart Fail. 2019 February; 21(2):163-171)). In addition, binding of the antibody to ADM leads to a prolongation of ADM's half-life. Even though NT-ADM antibodies partially inhibit ADM-mediated signalling, a large increase of circulating ADM results in an overall “net” increase of ADM activity in the blood compartment, where it exerts beneficial effects on ECs (predominantly barrier stabilization), whereas ADMs detrimental effects on VSMCs (vasodilation) in the interstitium are reduced.

The invention is not limited to the use of Adrecizumab specifically. There is no reason to doubt that what is true for Adrecizumab will also be true for antibodies sharing main essential features (in particular affinity and epitope specificity). Antibodies that target the same region must be expected to have the same technical effect, provided they have the same affinity and same or very comparable structural features (size, shape, etc.).

Throughout the specification the “antibodies”, or “antibody fragments” or “non-Ig scaffolds” in accordance with the invention are capable to bind ADM, and thus are directed against ADM, and thus can be referred to as “anti-ADM antibodies”, “anti-ADM antibody fragments”, or “anti-ADM non-Ig scaffolds”.

The term “antibody” generally comprises monoclonal and polyclonal antibodies and binding fragments thereof, in particular Fc-fragments as well as so called “single-chain-antibodies” (Bird et al. 1988), chimeric, humanized, in particular CDR-grafted antibodies, and dia or tetrabodies (Holliger et al. 1993). Also comprised are immunoglobulin-like proteins that are selected through techniques including, for example, phage display to specifically bind to the molecule of interest contained in a sample. In this context the term “specific binding” refers to antibodies raised against the molecule of interest or a fragment thereof. An antibody is considered to be specific, if its affinity towards the molecule of interest or the aforementioned fragment thereof is at least preferably 50-fold higher, more preferably 100-fold higher, most preferably at least 1000-fold higher than towards other molecules comprised in a sample containing the molecule of interest. It is well known in the art how to make antibodies and to select antibodies with a given specificity.

In one embodiment of the invention the anti-Adrenomedullin (ADM) antibody or anti-adrenomedullin antibody fragment or anti-ADM non-Ig scaffold is monospecific.

Monospecific anti-adrenomedullin (ADM) antibody or monospecific anti-adrenomedullin antibody fragment or monospecific anti-ADM non-Ig scaffold means that said antibody or antibody fragment or non-Ig scaffold binds to one specific region encompassing at least 5 amino acids within the target ADM. Monospecific anti-Adrenomedullin (ADM) antibody or monospecific anti-adrenomedullin antibody fragment or monospecific anti-ADM non-Ig scaffold are anti-adrenomedullin (ADM) antibodies or anti-adrenomedullin antibody fragments or anti-ADM non-Ig scaffolds that all have affinity for the same antigen. Monoclonal antibodies are monospecific, but monospecific antibodies may also be produced by other means than producing them from a common germ cell.

Said anti-ADM antibody or antibody fragment binding to ADM or non-Ig scaffold binding to ADM may be a non-neutralizing anti-ADM antibody or antibody fragment binding to ADM or non-Ig scaffold binding to ADM.

In a specific embodiment said anti-ADM antibody, anti-ADM antibody fragment or anti-ADM non-Ig scaffold is a non-neutralizing antibody, fragment or non-Ig scaffold. A neutralizing anti-ADM antibody, anti-ADM antibody fragment or anti-ADM non-Ig scaffold would block the bioactivity of ADM to nearly 100%, to at least more than 90%, preferably to at least more than 95%.

In contrast, a non-neutralizing anti-ADM antibody, or anti-ADM antibody fragment or anti-ADM non-Ig scaffold blocks the bioactivity of ADM less than 100%, preferably to less than 95%, preferably to less than 90%, more preferred to less than 80% and even more preferred to less than 50%. This means that bioactivity of ADM is reduced to less than 100%, to 95% or less but not more, to 90% or less but not more, to 80% or less but not more, to 50% or less but not more This means that the residual bioactivity of ADM bound to the non-neutralizing anti-ADM antibody, or anti-ADM antibody fragment or anti-ADM non-Ig scaffold would be more than 0%, preferably more than 5%, preferably more than 10%, more preferred more than 20%, more preferred more than 50%.

In this context (a) molecule(s), being it an antibody, or an antibody fragment or a non-Ig scaffold with “non-neutralizing anti-ADM activity”, collectively termed here for simplicity as “non-neutralizing” anti-ADM antibody, antibody fragment, or non-Ig scaffold, that e.g. blocks the bioactivity of ADM to less than 80%, is defined as
a molecule or molecules binding to ADM, which upon addition to a culture of an eukaryotic cell line, which expresses functional human recombinant ADM receptor composed of CRLR (calcitonin receptor like receptor) and RAMP3 (receptor-activity modifying protein 3), reduces the amount of cAMP produced by the cell line through the action of parallel added human synthetic ADM peptide, wherein said added human synthetic ADM is added in an amount that in the absence of the non-neutralizing antibody to be analyzed, leads to half-maximal stimulation of cAMP synthesis, wherein the reduction of cAMP by said molecule(s) binding to ADM takes place to an extent, which is not more than 80%, even when the non-neutralizing molecule(s) binding to ADM to be analyzed is added in an amount, which is 10-fold more than the amount, which is needed to obtain the maximal reduction of cAMP synthesis obtainable with the non-neutralizing antibody to be analyzed.

The same definition applies to the other ranges; 95%, 90%, 50% etc.

An antibody or fragment according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length.

Full-length immunoglobulin heavy chains are generally about 50 Kd or 446 amino acid in length. Light chains are encoded by a variable region gene at the NH₂-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia et al. 1987. Eur. J. Immunol. 17:105; Huston et al. 1988. Proc. Natd. Acad. Sci. U.S.A., 85:5879-5883; Bird et al. 1988. Science 242:423-426; Hood et al. 1984, Immunology, Benjamin, N.Y., 2nd ed.; Hunkapiller and Hood 1986. Nature 323:15-16). An immunoglobulin light or heavy chain variable region includes a framework region interrupted by three hypervariable regions, also called complementarity determining regions (CDR's) (see, Sequences of Proteins of Immunological Interest, E. Kabat et al. 1983, U.S. Department of Health and Human Services). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art, e.g., see U.S. Pat. No. 5,807,715. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDR's. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). A human antibody is an antibody wherein the light and heavy chain genes are of human origin. Human antibodies can be generated using methods known in the art. Human antibodies can be produced by immortalizing a human B cell secreting the antibody of interest. Immortalization can be accomplished, for example, by EBV infection or by fusing a human B cell with a myeloma or hybridoma cell to produce a trioma cell. Human antibodies can also be produced by phage display methods (see, e.g. WO91/17271; WO92/001047; WO92/20791), or selected from a human combinatorial monoclonal antibody library (see the Morphosys website). Human antibodies can also be prepared by using transgenic animals carrying a human immunoglobulin gene (for example, see WO93/12227; WO 91/10741).

Thus, the anti-ADM antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)₂-fragments, scFv-fragments, multimerized multivalent or/and multi-specific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulins and numerous others.

In addition to anti-ADM antibodies other biopolymer scaffolds are well known in the art to complex a target molecule and have been used for the generation of highly target specific biopolymers. Examples are aptamers, spiegelmers, anticalins and conotoxins. For illustration of antibody formats please see FIG. 1a, 1b and 1c.

In a preferred embodiment the anti-ADM antibody format is selected from the group comprising Fv fragment, scFv fragment, Fab fragment, scFab fragment, F(ab)₂ fragment and scFv-Fc Fusion protein.

In another preferred embodiment the antibody format is selected from the group comprising scFab fragment, Fab fragment, scFv fragment and bioavailability optimized conjugates thereof, such as PEGylated fragments. One of the most preferred formats is the scFab format.

Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US 2010/0028995), fibronectin scaffolds (e.g. described in EP 1 266 025; lipocalin-based scaffolds (e.g. described in WO 2011/154420); ubiquitin scaffolds (e.g. described in WO 2011/073214), transferrin scaffolds (e.g. described in US 2004/0023334), protein A scaffolds (e.g. described in EP 2 231 860), ankyrin repeat based scaffolds (e.g. described in WO 2010/060748), microproteins preferably microproteins forming a cysteine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1 941 867).

In one embodiment of the invention anti-ADM antibodies according to the present invention may be produced as outlined in Example 1 by synthesizing fragments of ADM as antigens. Thereafter, binder to said fragments are identified using the below described methods or other methods as known in the art.

Humanization of murine antibodies may be conducted according to the following procedure: For humanization of an antibody of murine origin the antibody sequence is analyzed for the structural interaction of framework regions (FR) with the complementary determining regions (CDR) and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro and Fransson 2008. Humanization of antibodies. Front Biosci. 2008 Jan. 1; 13:1619-33).

In a preferred embodiment the ADM antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is scFab format.

In another preferred embodiment, the anti-ADM antibody, anti-ADM antibody fragment, or anti-ADM non-Ig scaffold is a full-length antibody, antibody fragment, or non-Ig scaffold.

In a preferred embodiment the anti-ADM antibody or an anti-ADM antibody fragment or anti-ADM non-Ig scaffold is directed to and can bind to an epitope of at least 5 amino acids in length contained in ADM.

In a more preferred embodiment, the anti-ADM antibody or an anti-ADM antibody fragment or anti-ADM non-Ig scaffold is directed to and can bind to an epitope of at least 4 amino acids in length contained in ADM.

In one specific embodiment of the invention the anti-ADM antibody or anti-ADM antibody fragment binding to adrenomedullin or anti-ADM non-Ig scaffold binding to adrenomedullin is provided for use in therapy or prevention of shock in a patient, wherein said antibody or fragment or scaffold is not ADM-binding-Protein-1 (complement factor H).

In one specific embodiment of the invention the anti-Adrenomedullin (ADM) antibody or anti-ADM antibody fragment binding to adrenomedullin or anti-ADM non-Ig scaffold binding to adrenomedullin is provided for use in therapy or prevention of shock in a patient, wherein said antibody or fragment or scaffold binds to a region of preferably at least 4, or at least 5 amino acids within the sequence of amino acid 1-21 of mature human ADM: YRQSMNNFQGLRSFGCRFGTC SEQ ID No.: 22.

In a preferred embodiment of the present invention said anti-ADM antibody or anti-adrenomedullin antibody fragment or anti-ADM non-Ig scaffold binds to a region or epitope of ADM that is located in the N-terminal part (amino acid 1-21) of adrenomedullin.

In another preferred embodiment said anti-ADM-antibody or anti-ADM antibody fragment or anti-ADM non-Ig scaffold recognizes and binds to a region or epitope within amino acids 1-14 of adrenomedullin: YRQSMNNFQGLRSF (SEQ ID No.: 25) that means to the N-terminal part (amino acid 1-14) of adrenomedullin.

In another preferred embodiment said anti-ADM-antibody or anti-adrenomedullin antibody fragment or anti-ADM non-Ig scaffold recognizes and binds to a region or epitope within amino acids 1-10 of adrenomedullin: YRQSMNNFQG (SEQ ID No.: 26); that means to the N-terminal part (amino acid 1-10) of adrenomedullin.

In another preferred embodiment said anti-ADM antibody or anti-ADM antibody fragment or anti-ADM non-Ig scaffold recognizes and binds to a region or epitope within amino acids 1-6 of adrenomedullin: YRQSMN (SEQ ID No.: 27); that means to the N-terminal part (amino acid 1-6) of adrenomedullin. As stated above said region or epitope comprises preferably at least 4 or at least 5 amino acids in length.

In another preferred embodiment said anti-ADM antibody or anti-ADM antibody fragment or anti-ADM non-Ig scaffold recognizes and binds to the N-terminal end (amino acid 1) of adrenomedullin. N-terminal end means that the amino acid 1, that is “Y” of SEQ ID No. 20, 22 or 23, respectively and is mandatory for binding. The antibody or fragment or scaffold would neither bind N-terminal extended nor N-terminal modified Adrenomedullin nor N-terminal degraded adrenomedullin. This means in another preferred embodiment said anti-ADM-antibody or anti-ADM antibody fragment or anti-ADM non-Ig scaffold binds only to a region within the sequence of mature ADM if the N-terminal end of ADM is free. In said embodiment the anti-ADM antibody or anti-ADM antibody fragment or non-Ig scaffold would not bind to a region within the sequence of mature ADM if said sequence is e.g. comprised within pro-ADM.

For the sake of clarity, the numbers in brackets for specific regions of ADM like “N-terminal part (amino acid 1-21)” is understood by a person skilled in the art that the N-terminal part of ADM consists of amino acids 1-21 of the mature ADM sequence.

In another specific embodiment pursuant to the invention the herein provided anti-ADM antibody or anti-ADM antibody fragment or anti-ADM non-Ig scaffold does not bind to the C-terminal portion of ADM, i.e. the amino acid 43-52 of ADM: PRSKISPQGY-NH₂ (SEQ ID No.: 24).

An epitope, also known as antigenic determinant, is the part of an antigen that is recognized by the immune system, specifically by antibodies. For example, the epitope is the specific piece of the antigen to which an antibody binds. The part of an antibody that binds to the epitope is called a paratope. The epitopes of protein antigens are divided into two categories, conformational epitopes and linear epitopes, based on their structure and interaction with the paratope.

Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontiguous amino acid residues. A linear or a sequential epitope is an epitope that is recognized by antibodies by its linear sequence of amino acids, or primary structure and is formed by the 3-D conformation adopted by the interaction of contiguous amino acid residues.

In one specific embodiment it is preferred to use an anti-ADM antibody or anti-ADM antibody fragment or anti-ADM non-Ig scaffold according to the present invention, wherein said anti-ADM antibody or said anti-ADM antibody fragment or anti-ADM non-Ig scaffold leads to an increase of the ADM level or ADM immunoreactivity in serum, blood, plasma of at least 10%, preferably at least 50%, more preferably >50%, most preferably >100%.

In one specific embodiment it is preferred to use an anti-ADM antibody or anti-ADM antibody fragment or anti-ADM non-Ig scaffold according to the present invention, wherein said anti-ADM antibody or said anti-ADM antibody fragment or anti-ADM non-Ig scaffold is an ADM stabilizing antibody or an ADM stabilizing antibody fragment or an ADM stabilizing non-Ig scaffold that enhances the half-life (t_{1/2}; half retention time) of adrenomedullin in serum, blood, plasma at least 10%, preferably at least 50%, more preferably >50%, most preferably >100%.

The half-life (half retention time) of ADM may be determined in human serum, blood or plasma in absence and presence of an ADM stabilizing antibody or an ADM stabilizing antibody fragment or an ADM stabilizing non-Ig scaffold, respectively, using an immunoassay for the quantification of ADM.

The following steps may be conducted:
ADM may be diluted in human citrate plasma in absence and presence of an ADM stabilizing antibody or an adrenomedullin stabilizing antibody fragment or an adrenomedullin stabilizing non-Ig scaffold, respectively, and may be incubated at 24° C.
Aliquots are taken at selected time points (e.g. within 24 hours) and degradation of ADM may be stopped in said aliquots by freezing at −20° C.
The quantity of ADM may be determined by a hADM immunoassay directly, if the selected assay is not influenced by the stabilizing antibody. Alternatively, the aliquot may be treated with denaturing agents (like HCl) and, after clearing the sample (e.g. by centrifugation) the pH can be neutralized and the ADM-quantified by an ADM immunoassay. Alternatively, non-immunoassay technologies (e.g. RP-HPLC) can be used for ADM-quantification.
The half-life of ADM is calculated for ADM incubated in absence and presence of an ADM stabilizing antibody or an adrenomedullin stabilizing antibody fragment or an adrenomedullin stabilizing non-Ig scaffold, respectively.
The enhancement of half-life is calculated for the stabilized ADM in comparison to ADM that has been incubated in absence of an ADM stabilizing antibody or an adrenomedullin stabilizing antibody fragment or an adrenomedullin stabilizing non-Ig scaffold.

A two-fold increase of the half-life of ADM is an enhancement of half-life of 100%.

Half-life (half retention time) is defined as the period over which the concentration of a specified chemical or drug takes to fall to half its baseline concentration in the specified fluid or blood.

An assay that may be used for the determination of the half-life (half retention time) of adrenomedullin in serum, blood, plasma is described in Example 3.

In a preferred embodiment said anti-ADM antibody, anti-ADM antibody fragment or anti-ADM non-Ig scaffold is a non-neutralizing antibody, fragment or scaffold. A neutralizing anti-ADM antibody, anti-ADM antibody fragment or anti-ADM non-Ig scaffold would block the bioactivity of ADM to nearly 100%, to at least more than 90%, preferably to at least more than 95%. In other words, this means that said non-neutralizing anti-ADM antibody, anti-ADM antibody fragment or anti-ADM non-Ig scaffold blocks the bioactivity of ADM to less than 100%, preferably less than 95% preferably less than 90%.

In an embodiment wherein said non-neutralizing anti-ADM antibody, anti-ADM antibody fragment or anti-ADM non-Ig scaffold blocks the bioactivity of ADM to less than 95% an anti-ADM antibody, anti-ADM antibody fragment or anti-ADM non-Ig scaffold that would block the bioactivity of ADM to more than 95% would be outside of the scope of said embodiment. This means in one embodiment that the bioactivity is reduced to 95% or less but not more, preferably to 90% or less, more preferably to 80% or less, more preferably to 50% or less but not more.

In one embodiment of the invention the non-neutralizing antibody is an antibody binding to a region of at least 5 amino acids within the sequence of amino acid 1-21 of mature human ADM (SEQ ID No.: 14), or an antibody binding to a region of at least 5 amino acids within the sequence of amino acid 1-19 of mature murine ADM (SEQ ID No.: 17).

In another preferred embodiment of the invention the non-neutralizing antibody is an antibody binding to a region of at least 4 amino acids within the sequence of amino acid 1-21 of mature human ADM (SEQ ID No.: 14), or an antibody binding to a region of at least 5 amino acids within the sequence of amino acid 1-19 of mature murine ADM (SEQ ID No.: 17).

In a specific embodiment according to the present invention a non-neutralizing anti-ADM antibody or anti-ADM antibody fragment or ADM non-Ig scaffold is used, wherein said anti-ADM antibody or an anti-ADM antibody fragment blocks the bioactivity of ADM to less than 80%, preferably less than 50% (of baseline values). It has to be understood that said limited blocking of the bioactivity (meaning reduction of the bioactivity) of ADM occurs even at excess concentration of the antibody, fragment or scaffold, meaning an excess of the antibody, fragment or scaffold in relation to ADM. Said limited blocking is an intrinsic property of the ADM binder itself in said specific embodiment. This means that said antibody, fragment or scaffold has a maximal inhibition of 80% or 50% respectively. In a preferred embodiment said anti-ADM antibody, anti-ADM antibody fragment or anti-ADM non-Ig scaffold would block the bioactivity/reduce the bioactivity of anti-ADM to at least 5%. The stated above means that approximately 20% or 50% or even 95% residual ADM bioactivity remains present, respectively.

Thus, in accordance with the present invention the provided anti-ADM antibodies, anti-ADM antibody fragments, and anti-ADM non-Ig scaffolds do not neutralize the respective ADM bioactivity.

The bioactivity is defined as the effect that a substance takes on a living organism or tissue or organ or functional unit in vivo or in vitro (e.g. in an assay) after its interaction. In case of ADM bioactivity this may be the effect of ADM in a human recombinant ADM receptor cAMP functional assay. Thus, according to the present invention bioactivity is defined via an ADM receptor cAMP functional assay. The following steps may be performed in order to determine the bioactivity of ADM in such an assay:
Dose response curves are performed with ADM in said human recombinant ADM receptor cAMP functional assay.
The ADM concentration of half-maximal cAMP stimulation may be calculated.
At constant half-maximal cAMP-stimulating ADM concentrations dose response curves (up to 100 μg/ml final concentration) are performed by an ADM stabilizing antibody or ADM stabilizing antibody fragment or ADM stabilizing non-Ig scaffold, respectively.

A maximal inhibition in said ADM bioassay of 50% means that said anti-ADM antibody or said anti-ADM antibody fragment or said anti-ADM non-Ig scaffold, respectively, blocks the bioactivity of ADM to 50% of baseline values. A maximal inhibition in said ADM bioassay of 80% means that said anti-ADM antibody or said anti-adrenomedullin antibody fragment or said anti-adrenomedullin non-Ig scaffold, respectively, blocks the bioactivity of ADM to 80%. This is in the sense of blocking the ADM bioactivity to not more than 80%. This means approximately 20% residual ADM bioactivity remains present.

However, by the present specification and in the above context the expression “blocks the bioactivity of ADM” in relation to the herein disclosed anti-ADM antibodies, anti-ADM antibody fragments, and anti-ADM non-Ig scaffolds should be understood as mere decreasing the bioactivity of ADM from 100% to 20% remaining ADM bioactivity at maximum, preferably decreasing the ADM bioactivity from 100% to 50% remaining ADM bioactivity; but in any case there is ADM bioactivity remaining that can be determined as detailed above.

The bioactivity of ADM may be determined in a human recombinant Adrenomedullin receptor cAMP functional assay (Adrenomedullin Bioassay) according to Example 2.

In a preferred embodiment a modulating anti-ADM antibody or a modulating anti-ADM antibody fragment or a modulating anti-ADM non-Ig scaffold is used in therapy or prevention of shock in a patient.

A “modulating” anti-ADM antibody or a modulating anti-ADM antibody fragment or a modulating anti-ADM non-Ig scaffold is an antibody or antibody fragment or non-Ig scaffold that enhances the half-life (t_{1/2} half retention time) of adrenomedullin in serum, blood, plasma at least 10%, preferably at least, 50%, more preferably >50%, most preferably >100% and blocks the bioactivity of ADM to less than 80%, preferably less than 50% and said anti-ADM antibody, anti-ADM antibody fragment or anti-ADM non-Ig scaffold would block the bioactivity of ADM to at least 5%. These values related to half-life and blocking of bioactivity have to be understood in relation to the before-mentioned assays in order to determine these values. This is in the sense of blocking the ADM bioactivity of not more than 80% or not more than 50%, respectively.

Such a modulating anti-ADM antibody or modulating anti-ADM antibody fragment or a modulating anti-ADM non-Ig scaffold offers the advantage that the dosing of the administration is facilitated. The combination of partially blocking or partially reducing ADM bioactivity and increase of the in vivo half-life (increasing the ADM bioactivity) leads to beneficial simplicity of anti-ADM antibody or an anti-ADM antibody fragment or anti-ADM non-Ig scaffold dosing. In a situation of excess of endogenous ADM (maximal stimulation, late sepsis phase, shock, hypodynamic phase) the activity lowering effect is the major impact of the antibody or fragment or scaffold, limiting the (negative) effect of ADM. In case of low or normal endogenous ADM concentrations, the biological effect of anti-ADM antibody or anti-ADM antibody fragment or anti-ADM non-Ig scaffold is a combination of lowering (by partially blocking) and increase by increasing the ADM half-life. Thus, the non-neutralizing and modulating anti-ADM antibody or anti-ADM antibody fragment or anti-ADM non-Ig scaffold acts like an ADM bioactivity buffer in order to keep the bioactivity of ADM within a certain physiological range.

In a specific embodiment of the invention the antibody is a monoclonal antibody or a fragment thereof. In one embodiment of the invention the anti-ADM antibody or the anti-ADM antibody fragment is a human or humanized antibody or derived therefrom. In one specific embodiment one or more (murine) CDR's are grafted into a human antibody or antibody fragment.

Subject matter of the present invention in one aspect is a human or humanized CDR-grafted antibody or antibody fragment thereof that binds to ADM, wherein the human or humanized CDR-grafted antibody or antibody fragment thereof comprises an antibody heavy chain (H chain) comprising:

(SEQ ID No.: 1)
GYTFSRYW,
(SEQ ID No.: 2)
ILPGSGST
and/or
(SEQ ID No.: 3)
TEGYEYDGFDY

and/or further comprises an antibody light chain (L chain) comprising:

(SEQ ID No.: 4)
QSIVYSNGNTY,
RVS
(not part of the Sequencing Listing)
and/or
(SEQ ID No.: 5)
FQGSHIPYT.

In one specific embodiment of the invention subject matter of the present invention is a human or humanized monoclonal antibody that binds to ADM or an antibody fragment thereof that binds to ADM wherein the heavy chain comprises at least one CDR selected from the group comprising:

(SEQ ID No.: 1)
GYTFSRYW,
(SEQ ID No.: 2)
ILPGSGST
and/or
(SEQ ID No.: 3)
TEGYEYDGFDY

and wherein the light chain comprises at least one CDR selected from the group comprising:

(SEQ ID No.: 4)
QSIVYSNGNTY,
RVS
(not part of the Sequencing Listing)
and/or
(SEQ ID No.: 5)
FQGSHIPYT.

In a more specific embodiment of the invention subject matter of the invention is a human monoclonal antibody that binds to ADM or an antibody fragment thereof that binds to ADM wherein the heavy chain comprises the sequences:

(SEQ ID No.: 1)
GYTFSRYW,
(SEQ ID No.: 2)
ILPGSGST
and/or
(SEQ ID No.: 3)
TEGYEYDGFDY

and wherein the light chain comprises the sequences:

(SEQ ID No.: 4)
QSIVYSNGNTY,
RVS
(not part of the Sequencing Listing)
and/or
(SEQ ID No.: 5)
FQGSHIPYT.

In a very specific embodiment, the anti-ADM antibody has a sequence selected from the group comprising: SEQ ID No. 6, 7, 8, 9, 10, 11, 12, 13, 32 and 33.

The anti-ADM antibody or anti-ADM antibody fragment or anti-ADM non-Ig scaffold according to the present invention exhibits an affinity towards human ADM in such that affinity constant is greater than 10⁻⁷ M, preferred 10⁻⁸ M, preferred affinity is greater than 10⁻⁹ M, most preferred higher than 10⁻¹⁰ M. A person skilled in the art knows that it may be considered to compensate lower affinity by applying a higher dose of compounds and this measure would not lead out-of-the-scope of the invention. The affinity constants may be determined according to the method as described in Example 1.

Subject matter of the present invention is a human or humanized monoclonal antibody or fragment that binds to ADM or an antibody fragment thereof for use in therapy or prevention of shock in a patient according to the present invention, wherein said antibody or fragment comprises a sequence selected from the group comprising:

(AM-VH-C)
SEQ ID NO: 6
QVQLQQSGAELMKPGASVKISCKATGYTFSRYWIEWVKQRPGHGLEWIG
EILPGSGSTNYNEKFKGKATITADTSSNTAYMQLSSLTSEDSAVYYCTE
GYEYDGFDYWGQGTTLTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPK
(AM-VH1)
SEQ ID NO: 7
QVQLVQSGAEVKKPGSSVKVSCKASGYTFSRYWISWVRQAPGQGLEWMG
RILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPK
(AM-VH2-E40)
SEQ ID NO: 8
QVQLVQSGAEVKKPGSSVKVSCKASGYTFSRYWIEWVRQAPGQGLEWMG
RILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPK
(AM-VH3-T26-E55)
SEQ ID NO: 9
QVQLVQSGAEVKKPGSSVKVSCKATGYTFSRYWISWVRQAPGQGLEWMG
EILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPK
(AM-VH4-T26-E40-E55)
SEQ ID NO: 10
QVQLVQSGAEVKKPGSSVKVSCKATGYTFSRYWIEWVRQAPGQGLEWMG
EILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPK
(AM-VL-C)
SEQ ID NO: 11
DVLLSQTPLSLPVSLGDQATISCRSSQSIVYSNGNTYLEWYLQKPGQSP
KLLIYRVSNRFSGVPDRFSGSGSGTDFTLKISRVEAEDLGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(AM-VL1)
SEQ ID NO: 12
DVVMTQSPLSLPVTLGQPASISCRSSQSIVYSNGNTYLNWFQQRPGQSP
RRLIYRVSNRDSGVPDRFSGSGSGTDFTLKISRVEAEDVGVYYCFQGSH
IPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(AM-VL2-E40)
SEQ ID NO: 13
DVVMTQSPLSLPVTLGQPASISCRSSQSIVYSNGNTYLEWFQQRPGQSP
RRLIYRVSNRDSGVPDRFSGSGSGTDFTLKISRVEAEDVGVYYCFQGSH
IPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

Another embodiment of the invention relates to a human or humanized monoclonal antibody or fragment that binds to ADM or an antibody fragment thereof for use in therapy or prevention of shock in a patient, wherein said antibody or fragment comprises the following sequence as a heavy chain:

SEQ ID NO: 32
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFScSVMHEALHNHY
TQKSLSLSPGK

and comprises the following sequence as a light chain:

SEQ ID NO: 33
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC.

In a specific embodiment of the invention the antibody comprises the following sequence as a heavy chain:

SEQ ID NO: 32
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNH
YTQKSLSLSPGK

or a sequence that is >95% identical to it, preferably >98%, preferably >99% and comprises the following sequence as a light chain:

SEQ ID NO: 33
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC

or a sequence that is >95% identical to it, preferably >98%, preferably >99%.

To assess the identity between two amino acid sequences, a pairwise alignment is performed. Identity defines the percentage of amino acids with a direct match in the alignment.

In a specific embodiment of the invention the antibody comprises the following sequence as a heavy chain:

SEQ ID NO: 32
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNH
YTQKSLSLSPGK

or a sequence that comprises CDR-sequences 100% identical to SEQ ID No.: 1, SEQ ID No.: 2 and/or SEQ ID No.: 3 and is >95% identical to SEQ ID NO: 32, preferably >98%, preferably >99% and comprises the following sequence as a light chain:

SEQ ID NO: 33
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC

or a sequence that comprises CDR-sequences 100% identical to SEQ ID No.: 4 and/or SEQ ID No.: 5 and is >95% identical to SEQ ID NO: 33, preferably >98%, preferably >99%.

In embodiments of the present invention, the anti-ADM antibody or anti-ADM antibody fragment for use in the treatment or prevention of shock in a patient, may be administered in a dose of at least 0.5 mg/Kg body weight, particularly at least 1.0 mg/kg body weight, more particularly, from 1.0 to 20.0 mg/kg body weight, e.g., from 2.0 to 10 mg/kg body weight, from 2.0 to 8.0 mg/kg body weight, or from 2.0 to 5.0 mg/kg body weight.

The term “pharmaceutical formulation” as used herein refers to a preparation which is in such form as to permit the biological activity of an active ingredient contained therein to be effective, and which contains no additional components which are unacceptably toxic to a subject to which the formulation would be administered.

The present invention also relates to a pharmaceutical formulation comprising a therapeutically effective dose of the active ingredient, in combination with at least one pharmaceutically acceptable excipient.

“Pharmaceutically acceptable excipient” refers to an excipient that does not produce an adverse, allergic or other untoward reaction when administered to a subject. It includes in addition to a therapeutic protein, carriers, various diluents, fillers, salts, buffers, stabilizers, solubilizers, and other materials well known in the art. The characteristics of the carrier will depend on the route of administration.

With the above context, the following consecutively numbered embodiments provide further specific aspects of the invention:
1. Anti-adrenomedullin (ADM) antibody or anti-ADM antibody fragment or anti-ADM non-Ig scaffold for use in therapy or prevention of shock in a patient, wherein said patient is characterized by having a level of dipeptidyl peptidase 3 (DPP3) in a sample of bodily fluid below a threshold, wherein said threshold of DPP3 is </=40 ng/ml, or in the range between 22 ng/ml and 40 ng/ml, and said anti-ADM antibody or anti-ADM fragment or anti-ADM non-Ig scaffold binds to the N-terminal part (amino acid 1-21) of ADM: YRQSMNNFQGLRSFGCRFGTC (SEQ ID No. 14).
2. Anti-adrenomedullin (ADM) antibody or anti-ADM antibody fragment or anti-ADM non-Ig scaffold for use in therapy or prevention of shock in a patient according to embodiment 1, wherein said shock is selected from the group comprising shock due to hypovolemia, cardiogenic shock, obstructive shock and distributive shock, in particular cardiogenic shock or septic shock.
3. Anti-adrenomedullin (ADM) antibody or anti-ADM antibody fragment or anti-ADM non-Ig scaffold for use in therapy or prevention of shock in a patient according to embodiment 1 and 2, wherein
   in case of cardiogenic shock said patient may have suffered an acute coronary syndrome (e.g. acute myocardial infarction) or wherein said patient has heart failure (e.g. acute decompensated heart failure), myocarditis, arrhythmia, cardiomyopathy, valvular heart disease, aortic dissection with acute aortic stenosis, traumatic chordal rupture or massive pulmonary embolism, or
   in case of hypovolemic shock said patient may have suffered a hemorrhagic disease including gastrointestinal bleed, trauma, vascular etiologies (e.g. ruptured abdominal aortic aneurysm, tumor eroding into a major blood vessel) and spontaneous bleeding in the setting of anticoagulant use or a non-hemorrhagic disease including vomiting, diarrhea, renal loss, skin losses/insensible losses (e.g. burns, heat stroke) or third-space loss in the setting of pancreatitis, cirrhosis, intestinal obstruction, trauma, or
   in case of obstructive shock said patient may have suffered a cardiac tamponade, tension pneumothorax, pulmonary embolism or aortic stenosis, or
   in case of distributive shock said patient may have septic shock, neurogenic shock, anaphylactic shock or shock due to adrenal crisis.
4. Anti-adrenomedullin (ADM) antibody or anti-ADM antibody fragment or anti-ADM non-Ig scaffold for use in therapy or prevention of shock in a patient according to embodiments 1 to 3, wherein said threshold of DPP3 in a sample of bodily fluid of said patient is ≤30 ng/ml or in the range between 22 and 30 ng/mL.
5. Anti-adrenomedullin (ADM) antibody or anti-ADM antibody fragment or anti-ADM non-Ig scaffold for use in therapy or prevention of shock in a patient according to embodiment 4, wherein said threshold of DPP3 in a sample of bodily fluid of said patient is ≤25 ng/ml or in the range between 22 and 25 ng/mL.
6. Anti-adrenomedullin (ADM) antibody or anti-ADM antibody fragment or anti-ADM non-Ig scaffold for use in therapy or prevention of shock in a patient according to embodiments 1 to 5, wherein the level of DPP3 is determined by contacting said sample of bodily fluid with a capture binder that binds specifically to DPP3.
7. Anti-adrenomedullin (ADM) antibody or anti-ADM antibody fragment or anti-ADM non-Ig scaffold for use in therapy or prevention of shock in a patient according to embodiment 6, wherein the capture binder is an antibody.
8. Anti-adrenomedullin (ADM) antibody or anti-ADM antibody fragment or anti-ADM non-Ig scaffold for use in the treatment or prevention of shock in a patient according to embodiments 1 to 7, wherein either the level of DPP3 protein and/or the level of active DPP3 is determined and compared to a predetermined threshold.
9. Anti-adrenomedullin (ADM) antibody or anti-ADM antibody fragment or anti-ADM non-Ig scaffold for use in therapy or prevention of shock in a patient according to embodiments 1 to 8, wherein the level of DPP3 is determined with an immunoassay, in particular a sandwich immunoassay.
10. Anti-adrenomedullin (ADM) antibody or anti-ADM antibody fragment or anti-ADM non-Ig scaffold for use in therapy or prevention of shock in a patient according to embodiments 1 to 8, wherein said level of DPP3 is DPP3 activity, wherein the method for determining DPP3 activity in a bodily fluid sample of said subject comprises the steps:
   contacting said sample with a capture-binder that binds specifically to full-length DPP3,
   separating DPP3 bound to said capture binder,
   adding substrate of DPP3 to said separated DPP3,
   quantifying of said DPP3 activity by measuring and quantifying the conversion of a substrate of DPP3.
11. Anti-adrenomedullin (ADM) antibody or anti-ADM antibody fragment or anti-ADM non-Ig scaffold for use in therapy or prevention of shock in a patient according to embodiments 1 to 10, wherein said patient is additionally characterized by having a level of ADM-NH₂ above a threshold.
12. Anti-adrenomedullin (ADM) antibody or anti-ADM antibody fragment or anti-ADM non-Ig scaffold for use in therapy or prevention of shock in a patient according to embodiment 11, wherein said threshold of ADM-NH₂ in a sample of bodily fluid of said patient is between 40 and 100 pg/mL, more preferred between 50 and 90 pg/mL, even more preferred between 60 and 80 pg/mL, most preferred said threshold is 70 pg/mL.
13. Anti-adrenomedullin (ADM) antibody or anti-ADM antibody fragment or anti-ADM non-Ig scaffold for use in therapy or prevention of shock in a patient according to embodiment 12, wherein said threshold of ADM-NH₂ in a sample of bodily fluid of said patient is 70 pg/mL.
14. Anti-adrenomedullin (ADM) antibody or anti-ADM antibody fragment or anti-ADM non-Ig scaffold for use in therapy or prevention of shock in a patient according to embodiments 1 to 13, wherein the bodily fluid is selected from whole blood, plasma and serum.
15. Anti-adrenomedullin (ADM) antibody or anti-ADM antibody fragment or anti-ADM non-Ig scaffold for use in therapy or prevention of shock in a patient according to embodiment 14, wherein the bodily fluid is plasma.
16. Anti-adrenomedullin (ADM) antibody or anti-ADM antibody fragment or anti-ADM non-Ig scaffold for use in therapy or prevention of shock in a patient according to embodiments 11-15, wherein the level of ADM-NH₂ is determined by contacting said sample of bodily fluid with a capture binder that binds specifically to ADM-NH₂.
17. Anti-adrenomedullin (ADM) antibody or anti-ADM antibody fragment or anti-ADM non-Ig scaffold for use in therapy or prevention of shock in a patient according to embodiment 16, wherein the capture binder is an antibody.
18. Anti-adrenomedullin (ADM) antibody or anti-ADM antibody fragment or anti-ADM non-Ig scaffold for use in therapy or prevention of shock in a patient according to embodiments 1 to 17, wherein said anti-ADM antibody or anti-ADM antibody fragment or anti-ADM non-Ig scaffold recognizes and binds to the N-terminal end (amino acid 1) of ADM-Gly and/or ADM-NH₂.
19. Anti-adrenomedullin (ADM) antibody or anti-ADM antibody fragment or anti-ADM non-Ig scaffold for use in therapy or prevention of shock in a patient according to embodiments 1 to 18, wherein said antibody, antibody fragment or non-Ig scaffold does not bind to the C-terminal portion of ADM, having the sequence amino acid 43-52 of ADM: PRSKISPQGY-NH₂ (SEQ ID NO: 24).
20. Anti-adrenomedullin (ADM) antibody or anti-ADM antibody fragment or anti-ADM non-Ig scaffold for use in therapy or prevention of shock in a patient according to embodiments 1 to 19, wherein said antibody or fragment or scaffold blocks the bioactivity of ADM not more than 80%, preferably not more than 50%.
21. Anti-adrenomedullin (ADM) antibody or anti-ADM antibody fragment or anti-ADM non-Ig scaffold for use in therapy or prevention of shock in a patient according to embodiments 1 to 20, wherein said antibody or fragment is a monoclonal antibody or fragment that binds to ADM or an antibody fragment thereof, wherein the heavy chain comprises the sequences:

CDR1:
SEQ ID NO: 1
GYTFSRYW
CDR2:
SEQ ID NO: 2
ILPGSGST
CDR3:
SEQ ID NO: 3
TEGYEYDGFDY

and wherein the light chain comprises the sequences:

CDR1:
SEQ ID NO: 4
QSIVYSNGNTY
CDR2:
RVS
CDR3:
SEQ ID NO: 5
FQGSHIPYT.

22. Anti-adrenomedullin (ADM) antibody or anti-ADM antibody fragment or anti-ADM non-Ig scaffold for use in therapy or prevention of shock in a patient according to embodiments 1 to 21, wherein said antibody or fragment comprises a sequence selected from the group comprising as a VH region:

(AM-VH-C)
SEQ ID NO: 6
QVQLQQSGAELMKPGASVKISCKATGYTFSRYWIEWVKQRPGHGLEWIGEILPGSGST
NYNEKFKGKATITADTSSNTAYMQLSSLTSEDSAVYYCTEGYEYDGFDYWGQGTTLTV
SSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPK
(AM-VH1)
SEQ ID NO: 7
QVQLVQSGAEVKKPGSSVKVSCKASGYTFSRYWISWVRQAPGQGLEWMGRILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPK
(AM-VH2-E40)
SEQ ID NO: 8
QVQLVQSGAEVKKPGSSVKVSCKASGYTFSRYWIEWVRQAPGQGLEWMGRILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPK
(AM-VH3-T26-E55)
SEQ ID NO: 9
QVQLVQSGAEVKKPGSSVKVSCKATGYTFSRYWISWVRQAPGQGLEWMGEILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPK
(AM-VH4-T26-E40-E55)
SEQ ID NO: 10
QVQLVQSGAEVKKPGSSVKVSCKATGYTFSRYWIEWVRQAPGQGLEWMGEILPGSGS
TNYAQKFQGRVTITADESTSTAYMELSSLRSEDTAVYYCTEGYEYDGFDYWGQGTTVT
VSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKRVEPK

and comprises a sequence selected from the group comprising the following sequence as a VL region:

(AM-VL-C)
SEQ ID NO: 11
DVLLSQTPLSLPVSLGDQATISCRSSQSIVYSNGNTYLEWYLQKPGQSPKLLIYRVSNRF
SGVPDRFSGSGSGTDFTLKISRVEAEDLGVYYCFQGSHIPYTFGGGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
(AM-VL1)
SEQ ID NO: 12
DVVMTQSPLSLPVTLGQPASISCRSSQSIVYSNGNTYLNWFQQRPGQSPRRLIYRVSNRD
SGVPDRFSGSGSGTDFTLKISRVEAEDVGVYYCFQGSHIPYTFGQGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
(AM-VL2-E40)
SEQ ID NO: 13
DVVMTQSPLSLPVTLGQPASISCRSSQSIVYSNGNTYLEWFQQRPGQSPRRLIYRVSNRD
SGVPDRFSGSGSGTDFTLKISRVEAEDVGVYYCFQGSHIPYTFGQGTKLEIKRTVAAPSV
FIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC.

23. Anti-adrenomedullin (ADM) antibody or anti-ADM antibody fragment or anti-ADM non-Ig scaffold for use in therapy or prevention of shock in a patient according to embodiments 1 to 22, wherein said antibody or fragment comprises the following sequence as a heavy chain:

SEQ ID NO: 32
QVQLVQSGAEVKKPGSSVKVSCKASGYTFSRYWIEWVRQAPGQGLEWIGEILPGSGSTN
YNQKFQGRVTITADTSTSTAYMELSSLRSEDTAVYYCTEGYEYDGFDYWGQGTTVTVSS
ASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQ
QGNVFSCSVMHEALHNHYTQKSLSLSPGK

or a sequence that is >95% identical to it,
and comprises the following sequence as a light chain:

SEQ ID NO: 33
DVVLTQSPLSLPVTLGQPASISCRSSQSIVYSNGNTYLEWYLQRPGQSPRLLIYRVSNRFSG
VPDRFSGSGSGTDFTLKISRVEAEDVGVYYCFQGSHIPYTFGGGTKLEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC

or a sequence that is >95% identical to it.
24. Anti-adrenomedullin (ADM) antibody or anti-ADM antibody fragment or anti-ADM non-Ig scaffold for use in therapy or prevention of shock in a patient according to embodiments 1-23, wherein the anti-adrenomedullin (ADM) antibody or anti-ADM antibody fragment or anti-ADM non-Ig scaffold binds to the N-terminal part (amino acid 1-10) of ADM: YRQSMNNFQG (SEQ ID No. 26).
25. Anti-adrenomedullin (ADM) antibody or an anti-adrenomedullin antibody fragment or anti-ADM non-Ig scaffold for use in therapy according to any of embodiments 1-24, wherein said antibody or fragment or scaffold exhibits a binding affinity to ADM of at least 10⁻⁷ M by label-free surface plasmon resonance using a Biacore 2000 system.
26. Anti-adrenomedullin (ADM) antibody or anti-ADM antibody fragment or anti-ADM non-Ig scaffold for use in therapy or prevention of shock in a patient according to embodiment 25, wherein the anti-ADM antibody or anti-ADM antibody fragment or anti-ADM non-Ig scaffold exhibits an affinity towards human ADM is between 1×10⁻⁹ to 3×10⁻⁹ M by label-free surface plasmon resonance using a Biacore 2000 system.
27. Anti-adrenomedullin (ADM) antibody or anti-ADM antibody fragment or anti-ADM non-Ig scaffold for use in therapy or prevention of shock in a patient according to embodiments 1-26, wherein the anti-ADM antibody or anti-ADM antibody fragment is an IgG1 antibody.
28. Pharmaceutical formulation for use in therapy or prevention of shock of a patient comprising an anti-adrenomedullin (ADM) antibody or an anti-ADM antibody fragment or anti-ADM non-Ig scaffold according to any of embodiments 1-27.
29. Pharmaceutical formulation for use in therapy or prevention of shock of a patient according to embodiment 28, wherein said pharmaceutical formulation is a solution, preferably a ready-to-use solution.
30. Pharmaceutical formulation for use in therapy or prevention of shock of a patient according to embodiments 28 and 29, wherein said pharmaceutical formulation is in a freeze-dried state.
31. Pharmaceutical formulation for use in therapy or prevention of shock of a patient according to embodiments 28 and 30, wherein said pharmaceutical formulation is administered intra-muscular.
32. Pharmaceutical formulation for use in therapy or prevention of shock of a patient according to embodiments 28 and 31, wherein said pharmaceutical formulation is administered intra-vascular.
33. Pharmaceutical formulation for use in therapy or prevention of shock of a patient according to embodiments 28 and 32, wherein said pharmaceutical formulation is administered via infusion.
34. Pharmaceutical formulation for use in therapy or prevention of shock of a patient according to embodiments 28 and 33, wherein said pharmaceutical formulation is to be administered systemically.

### Methods for Obtaining Monoclonal Antibodies

In all of the following embodiments, the term monoclonal antibody is meant to include monoclonal antibodies, as well as fragments of monoclonal antibodies, such as the ones detailed herein, more particularly monoclonal antibodies.

### Hybridoma

In a further aspect, the antibody according to the present invention is a monoclonal antibody obtainable by a method comprising:
i) fusing antibody-secreting cells from an animal previously immunized with an antigen with myeloma cells to obtain a multitude of hybridomas,
ii) isolating from said multitude of hybridomas a hybridoma producing a desired monoclonal antibody.

In certain embodiments, the antibody according to the present invention is a monoclonal antibody obtainable by isolating from a multitude of hybridomas a hybridoma producing a desired monoclonal antibody, wherein said multitude of hybridomas were produced by fusing antibody-secreting cells from an animal previously immunized with an antigen with myeloma cells to obtain multitude of hybridomas.

A desired monoclonal antibody is in particular a monoclonal antibody binding the antigen, in particular with a binding affinity of at least 10⁷ M⁻¹, preferred 10⁸ M⁻¹, more preferred affinity is greater than 10⁹ M⁻¹, most preferred greater than 10¹⁰ M⁻¹.

In certain embodiments of the method for obtaining an antibody, in step i) the animal is a mammal, particularly a rabbit, a mouse or a rat, more particularly a mouse, more particularly a Balb/c mouse.

In certain embodiments of the method for obtaining an antibody, in step i) the antibody-secreting cell is a splenocyte, more particularly an activated B-cell.

In certain embodiments of the method for obtaining an antibody, in step i) fusing involves the use of polyethylene glycol.

In certain embodiments of the method for obtaining an antibody, in step i) the myeloma is derived from a mammal, in certain embodiments from the same species of mammal from which the multitude of antibody-secreting cells is obtained. In certain specific embodiments of the method for obtaining an antibody, in step i) the myeloma cells are of the cell line SP2/0.

In certain embodiments of the method for obtaining an antibody, said fusing in step i) comprises PEG-assisted fusion, Sendai virus-assisted fusion or electric current-assisted fusion.

In certain embodiments of the method for obtaining an antibody, said isolating in step ii) comprises performing an antibody capture assay, an antigen capture assay, and/or a functional screen.

In certain embodiments of the method for obtaining an antibody, in step ii) isolating the hybridoma producing a desired monoclonal antibody may involve cloning and re-cloning the hybridomas using the limiting-dilution technique.

In one embodiment, said antibody capture assay comprises
a) binding an antigen to a substrate, particularly a solid substrate,
b) allowing the produced antibodies to bind to the antigen,
c) removing unbound antibodies by washing,
d) detecting bound antibodies.

In one embodiment, said antigen capture assay comprises
a) binding the produced antibodies to a substrate, particularly a solid substrate,
b) allowing antigen to bind to said antibodies,
c) removing unbound antigen by washing,
d) detecting bound antigen;
or said antigen capture assay comprises
a) allowing an antigen to bind the produced antibodies to form an antibody-antigen complex,
b) binding said antibody-antigen complex to a substrate, particularly a solid substrate,
c) removing unbound antigen by washing,
d) detecting bound antigen.

In one embodiment, said isolating of step ii) comprises performing an enzyme-linked immunosorbent assay, fluorescence-activated cell sorting, cell staining, immunoprecipitation, and/or a western blot.

In one embodiment, said detecting of the antibody or the antigen is accomplished with an immunoassay.

In one embodiment, the animal is a transgenic animal, in particular a transgenic mouse (wherein in particular the mouse immunoglobulin (Ig) gene loci have been replaced with human loci within the transgenic animal genome), such as HuMabMouse or XenoMouse.

In one embodiment, the antigen comprises a peptide as described herein in Table 1, or Table 6 respectively, which in certain embodiments (in particular for immunization) may be conjugated to a protein, particularly a serum protein, more particularly a serum albumin, more particularly BSA.

In a preferred embodiment, the antibody according to the present invention is a monoclonal antibody obtainable by a method comprising:
i) fusing splenocytes cells from a Balb/c mouse previously immunized with a peptide as described herein in Table 1 or 6 with SP2/0 myeloma cells using polyethylene glycol, to obtain a multitude of hybridomas,
ii) isolating from said multitude of hybridomas a hybridoma producing a desired monoclonal antibody;
   more preferably, the method comprises
growing hybridomas for a first period (in particular 2 weeks) in HAT medium [RPMI 1640 culture medium supplemented with 20% fetal calf serum and HAT-Supplement]
followed replacing HAT medium with HT Medium for a multitude of passages (in particular 3)
followed by returning to the normal cell culture medium for a second time period, in particular until the end of three weeks after fusion
primary screening of Cell culture supernatants for antigen-specific IgG antibodies
propagating microcultures of cells that tested positive in 4)
retesting Cell culture supernatants of microcultures for antigen-specific IgG antibodies
cloning and re-cloning cultures that tested positive in 6), using the limiting-dilution technique
optionally determining the isotypes of clones obtained from 7)
optionally purifying antibodies via Protein A

### Phage Display

In a further aspect, the antibody according to the present invention is a monoclonal antibody obtainable by a method comprising:
i) isolating at least one antibody having affinity to an antigen from an antibody gene library;
ii) generating at least one cell strain expressing said at least one antibody;
iii) isolating the at least one antibody from a culture of the at least one cell strain obtained in step ii).

An antibody having affinity to an antigen is in particular an antibody with a binding affinity of at least 10⁷ M⁻¹, preferred 10⁸ M⁻¹, more preferred affinity is greater than 10⁹ M⁻¹, most preferred greater than 10¹⁰ M⁻¹.

In a certain embodiment, the antibody according to the present invention is a monoclonal antibody obtainable by isolating at least one antibody from a culture derived from at least one cell strain which expressed at least one antibody having affinity to an antigen from an antibody gene library.

In one embodiment, the antigen comprises a peptide as described herein in Table 1, or Table 6 respectively, which in certain embodiments may be bound to a solid phase.

In certain embodiments of the method for obtaining an antibody, in step i) the antibody gene library is a naive antibody gene library, particularly a human naive antibody gene library, more particularly in said library the antibodies are presented via phage display, i.e. on phages comprising a nucleotide sequence encoding for such respective antibody; more particularly the library HAL 7, HAL 8, or HAL 9, more particularly a library comprising the human naive antibody gene libraries HAL7/8.

In certain embodiments of the method for obtaining an antibody, in step i) screening comprises the use of an antigen, particularly an antigen containing a tag, more particularly a biotin tag, linked thereto via two different spacers. In particular embodiments, such panning strategy includes a mix of panning rounds with non-specifically bound antigen and antigen bound specifically via the tag, in the case of a biotin tag, bound to streptavidin. In this way, the background of non-specific binders may be minimized.

In certain embodiments of the method for obtaining an antibody, in step i), in embodiments wherein the library is a phage display library, the antibody is isolated by isolating a phage presenting said antibody (and comprising a nucleotide sequence encoding for the antibody).

In certain embodiments of the method for obtaining an antibody, in step ii) said cell strain is generated via introduction of a nucleotide sequence encoding for the antibody), in embodiments wherein the library in step i) is a phage display library, the isolated phage from step i) may be used to produce a bacterial strain, e.g. an E. coli strain, expressing the antibody.

In certain embodiments of the method for obtaining an antibody, in step iv); in embodiments wherein the library in step i) is a phage display library and wherein a bacterial strain is produced in step ii), antibody may be isolated from the supernatant of the culture.

It is understood that, as used in describing the methods for obtaining an antibody, the term “one antibody” in the expression “at least one antibody” in particular may include more than one antibody molecule of antibodies having the same amino acid sequence. This understanding applies, mutatis mutandis, to the term “one cell strain”.

In certain embodiments of the method for obtaining an antibody, more than one antibody (referring to a multitude of antibodies having distinct amino acid sequences, respectively) is isolated in step i) and accordingly more than one cell strain is generated in step ii). Such method may involve the selection of clones that are positive for binding to the antigen, e.g. via a binding assay, e.g. an ELISA assay involving the antigen, and cells positive for binding to the antigen may be isolated to produce monoclonal cell strains.

In a preferred embodiment, the antibody according to the present invention is a monoclonal antibody obtainable by a method comprising:
i) isolating at least one antibody having affinity to an antigen from an antibody gene library comprising the human naive antibody gene libraries HAL7/8, by eluting phages carrying said antibody from the library;
ii) generating at least one E. coli cell strain expressing said at least one antibody;
iii) isolating the at least one antibody from the supernantant a culture of the at least one E. coli cell strain obtained in step ii).

In a further aspect, an antibody fragment according to the present invention is produced by a method in volving enzymatic digestion of an antibody. In certain embodiments, this method produces e.g. Fab or F(ab)2 antibody fragments. In certain embodiments, this method involves digestion with pepsin or papain, which are optionally immobilized on a surface.

In certain embodiments, antibodies may be humanized by CDR-grafting, in particular by a process involving the steps:
extracting RNA from hybridomas expressing an antibody of interest (e.g. obtained by a method as described herein);
amplifying said extracted RNA via RT-PCR, in particular with primer sets specific for the heavy and light chains of the antibody of interest, to obtain to obtain a DNA product;
further amplifying said DNA product via PCR, in particular using semi-nested primer sets specific for antibody variable regions;
determining the sequence of the DNA product;
aligning said sequence with homologous human framework sequences to determine a humanized sequence for the variable heavy chain and the variable light chain sequences (of the desired antibody).

In certain embodiments, antibodies may be humanized by aligning the sequence of a DNA product that was obtained by amplifying RNA extracted from hybridomas expressing an antibody of interest via RT-PCR, in particular with primer sets specific for the heavy and light chains of the antibody of interest and further amplifying the DNA obtained therefrom via PCR, in particular using semi-nested primer sets specific for antibody variable regions, with homologous human framework sequences to determine a humanized sequence for the variable heavy chain and the variable light chain sequences (of the desired antibody).

In certain embodiments, antibodies may be humanized by
determining the complementary determining regions (CDR), which may be accomplished by analyzing the structural interaction of framework regions (FR) with the complementary determining regions (CDR) and the antigen;
translplanting said CDR sequences into a human framework region.

In certain embodiments, antibodies may be humanized by translplanting CDR sequences, which may preferably have been determined by analyzing the structural interaction of framework regions (FR) with the complementary determining regions (CDR) and the antigen, into a human framework region.

In certain embodiments variations in the amino acid sequence of the CDRs or FRs may be introduced to maintain structural interactions with the antigen (which may otherwise be abolished by introducing the human FR sequences), for instance by a random approach using phage display libraries or via directed approach guided by molecular modeling.

The DNA sequences encoding for antibodies determined as detailed herein can be transferred by known genetic engineering techniques into cells and used for production of the antibody.

### Producing Antibodies

In a further aspect, the antibody according to the present invention is a monoclonal antibody obtainable by the methods described herein, produced by a method comprising:
culturing a cell strain comprising a nucleotide sequence encoding for the antibody;
isolating the antibody from said culture.

In a further certain aspect, the antibody according to the present invention is a monoclonal antibody obtainable by the methods described herein, produced by isolating the antibody from a culture of a cell strain comprising a nucleotide sequence encoding for said antibody.

In certain embodiments of said method, the cell strain is produced as described herein above and may comprise bacterial cells, such as gram-negative bacteria, e.g. E. coli, Proteus mirabilis, or Pseudomonas putidas, gram-positive bacteria, e.g. Bacillus brevis, Bacillus subtilis, Bacillus megaterium, Lactobacilli such as Lactobacillus zeae/casei or Lactobacillus paracasei, or Streptomyces, such as Streptomyces lividans; eucariotic cells such as yest, e.g. Pichia pastoris, Saccharomyces cerevisiae, Hansenula polymorpha, Schizosaccharomyces pombe, Schwanniomyces occidentalis, Kluyveromyces lactis, or Yarrowia lipolytica; fugi, such as filamentous fungi, e.g. of the genus Trichoderma of Aspergillus, such as A. niger (e.g. subgenus A. awamori) and Aspergillus oryzae, Trichoderma reesei, Chrysosporium, such as C. lucknowense; protozoae, such as Leishmania, e.g. L. tarentolae; insect cells, such as insect cells transfected a Baculovirus, e.g. AcNPV, such as insect cell lines from Spodoptera frugiperda, e.g. Sf-9 or Sf-21, Drosophila melanogaster, e.g. DS2, or Trichopulsia ni, e.g. High Five cells (BTI-TN-5B1-4); mammalian cells such as hamster, e.g. Chinese hamster ovary such as K1−, DukX B11−, DG44, Lec13, or BHK, mouse, e.g. mouse myeloma such as NSO, Homo sapiens, e.g. Per.C6, AGE1.HN, HEK293.

In certain embodiments of said method, the cells may be hybridoma cells, e.g. as described herein.

In certain embodiments of said method, culturing may take place in a static suspension culture, an agitated suspension culture, a membrane-based culture, a matrix-based culture or a high cell density bioreactor; a vessel for such culturing may be selected from the group comprising a T-flask, a roller culture, a spinner culture, a stirred tank bioreactor, an airlift bioreactor, a static membrane-based or matrix-based culture system, a suspension bioreactor, a fluidized bed bioreactor, a ceramic bioreactor, a perfusion system, a hollow fiber bioreactor.

In certain embodiments of said method, the cells may be immobilized on a matrix.

A high cell density bioreactor is in particular a culture system capable of generating cell densities greater than 10\hat{ }8 cells/ml.

In a further aspect, the antibody according to the present invention is a monoclonal antibody obtainable by the methods described herein, produced by a method comprising:
generating a transgenic plant or animal comprising a nucleotide sequence encoding for the antibody;
isolating the antibody from said plant or animal or a secretion or product of said plant or animal.

In a certain further aspect, the antibody according to the present invention is a monoclonal antibody obtainable by the methods described herein, produced by isolating the antibody from a transgenic plant or transgenic animal or a secretion or product of a transgenic plant or transgenic animal having a nucleotide sequence encoding for the antibody.

Said animal may e.g. be selected from a chicken, a mouse, a rat, a rabbit, a cow, a goat, a sheep, a pig; said secretion or product may e.g. be milk or an egg. Said plant may e.g. be selected from tobacco (N. tabacum or N. benthamiana), duckweed (Lemna minor), Chlamydomonas reinhardtii, rice, Arabidopsis thaliana, alfalfa (Medicago sativa), lettuce, maize.

The antibodies can in certain embodiments be isolated by physicochemical fractionation, e.g. size exclusion chromatography, precipitation, e.g. using ammonium sulphate, ion exchange chromatography, immobilized metal chelate chromatography gel filtration, zone electrophoresis; based on their classification e.g. binding to bacterial proteins A, G, or L, jacalin; antigen-specific affinity purification via immobilized ligands/antigens; if necessary, low molecular weight components can be removed by methods like dialysis, desalting, and diafiltration.

In some embodiments the antibody is encoded by a nucleotide sequence where the nucleotide sequence is a reverse transcription of an amino acid sequence from an antibody produced by one of the processes described herein.

### FIGURE DESCRIPTION

FIG. 1a: Illustration of antibody formats—Fv and scFv-Variants.

FIG. 1b: Illustration of antibody formats—heterologous fusions and bifunctional antibodies.

FIG. 1c: Illustration of antibody formats—bivalental antibodies and bispecific antibodies.

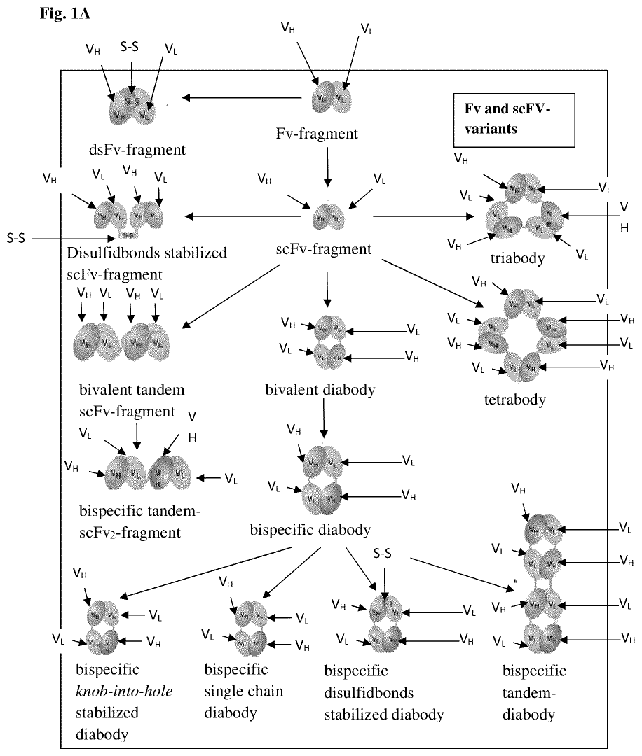

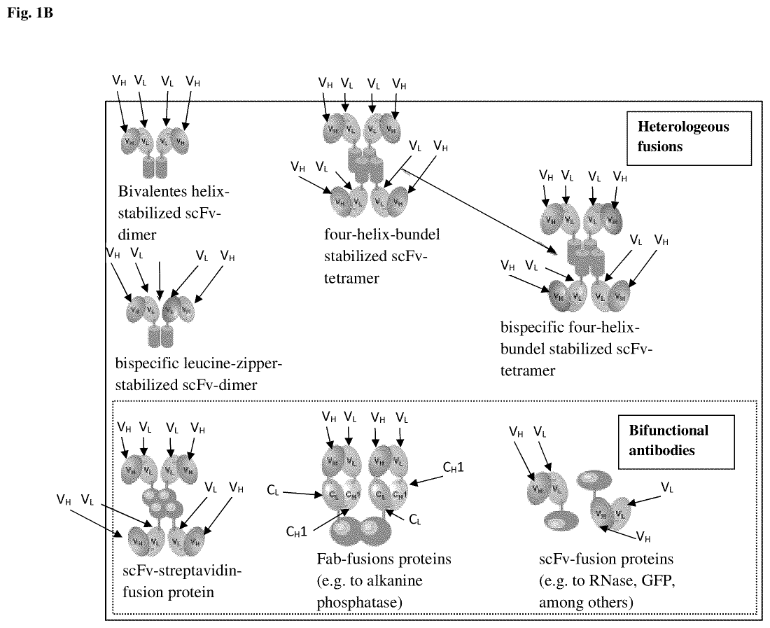

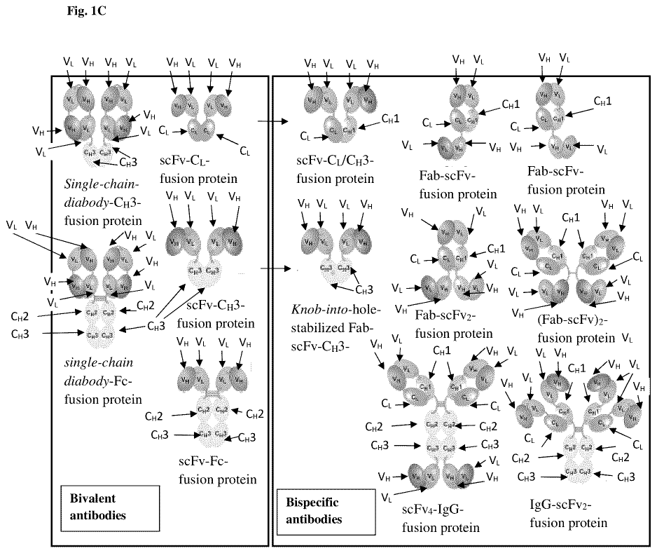

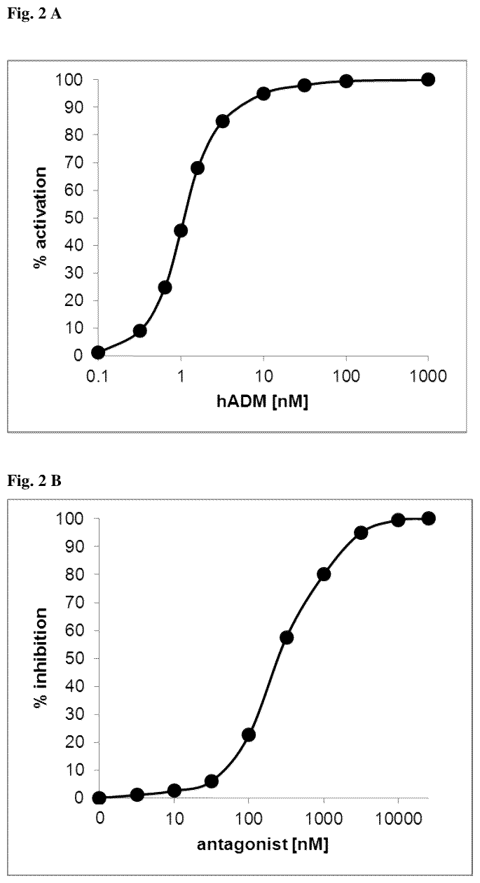

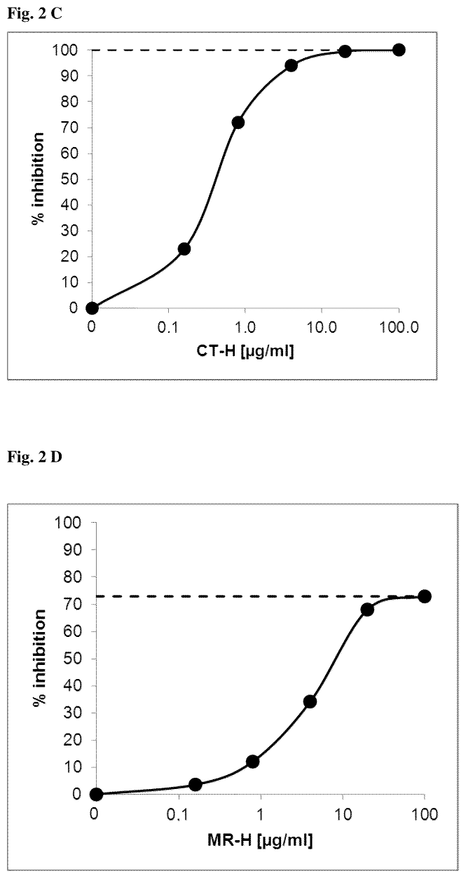

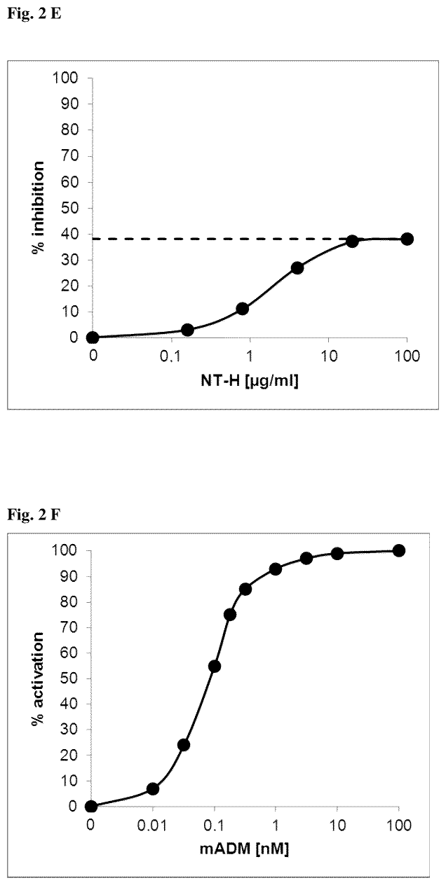

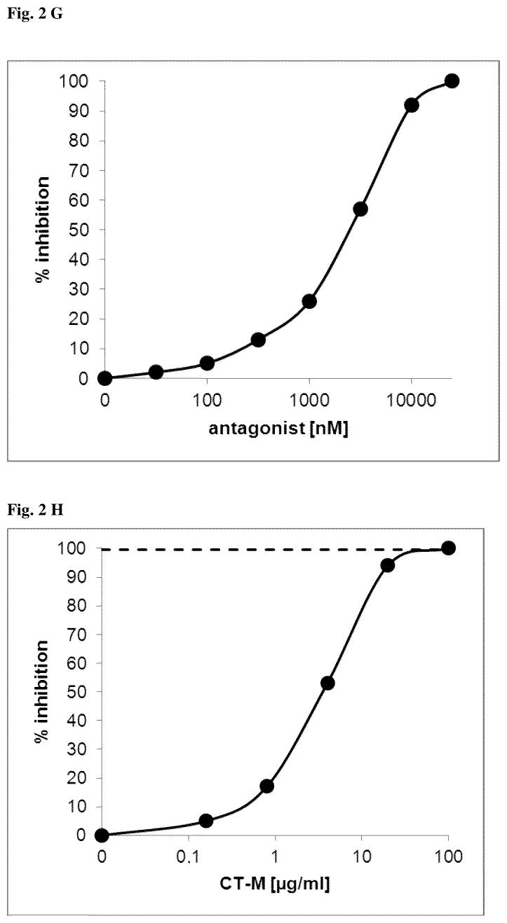

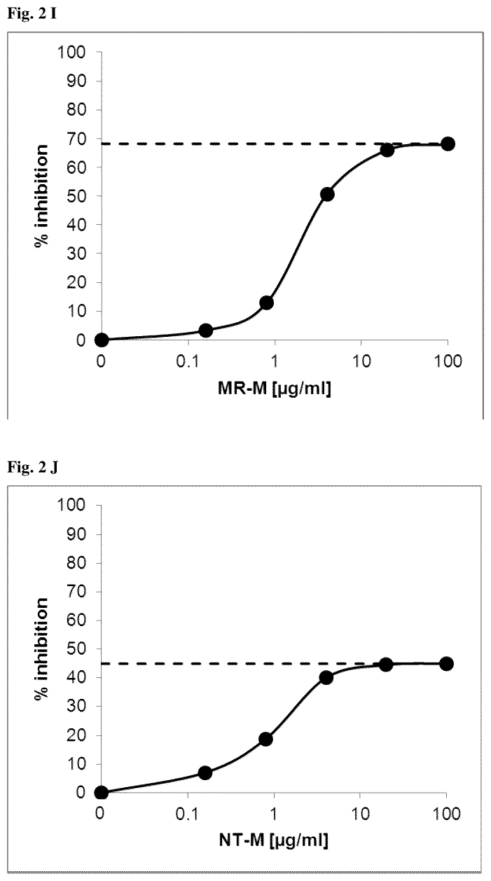

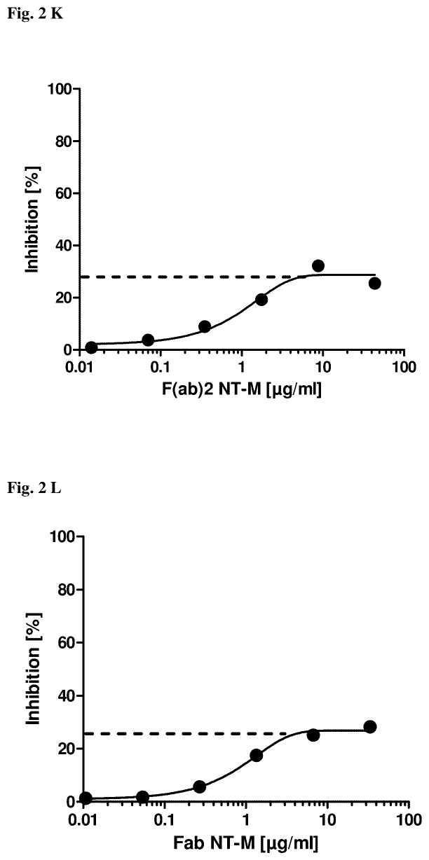

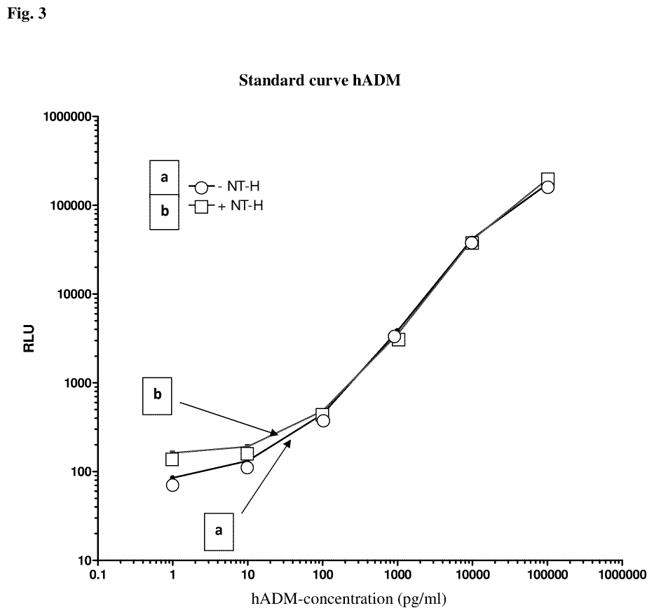

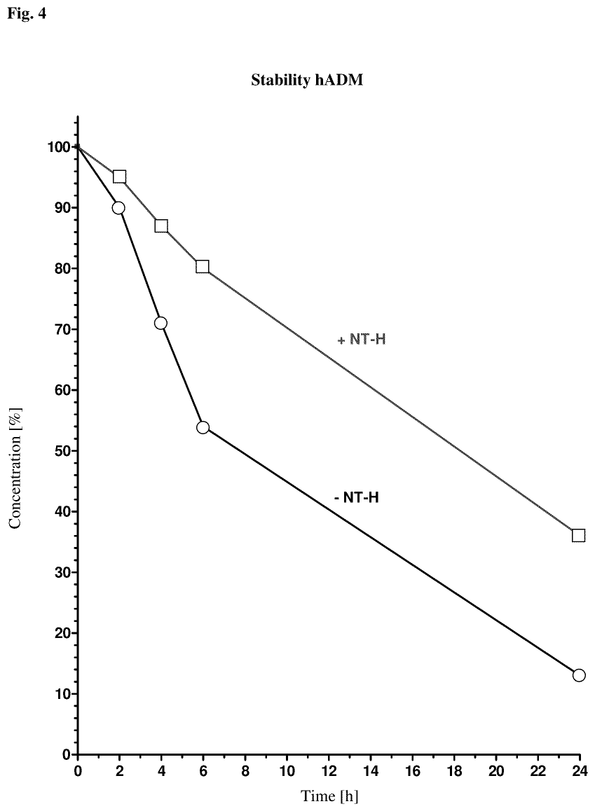

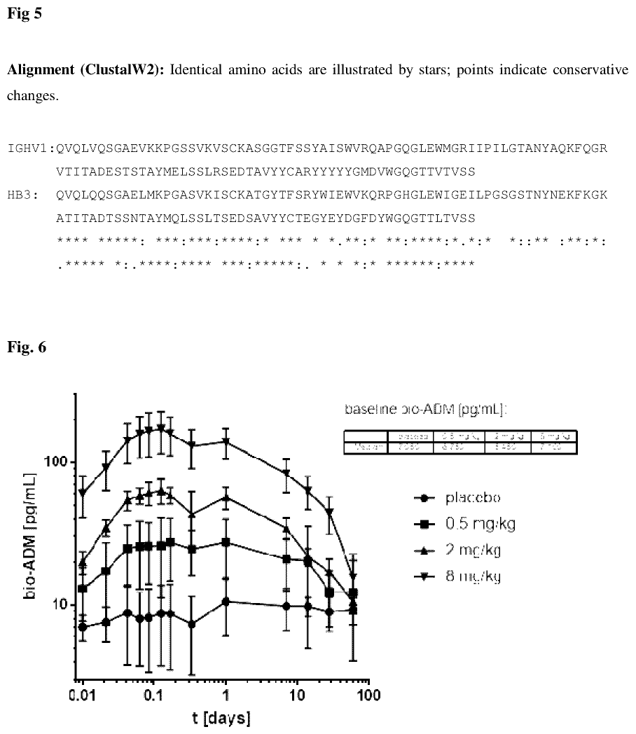

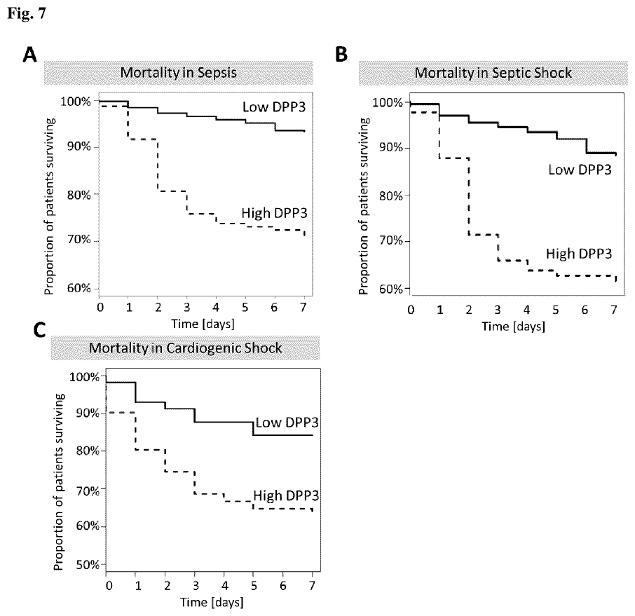

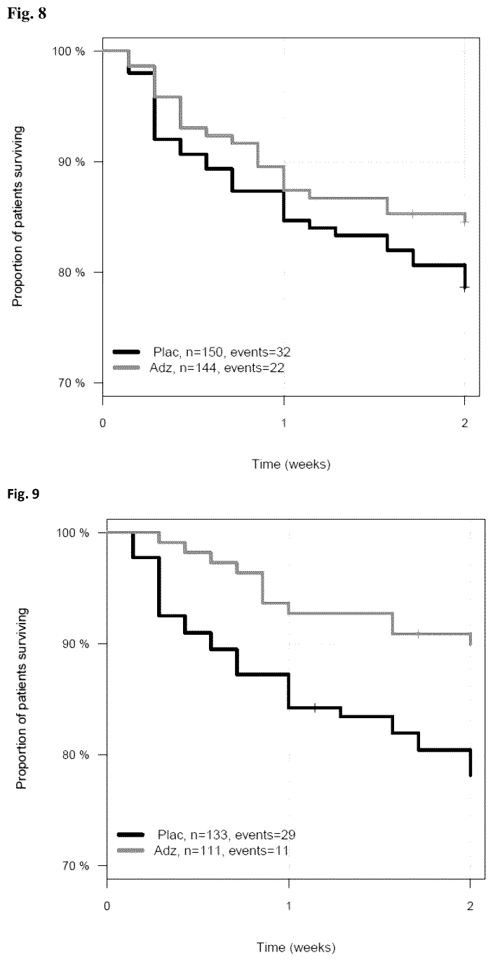

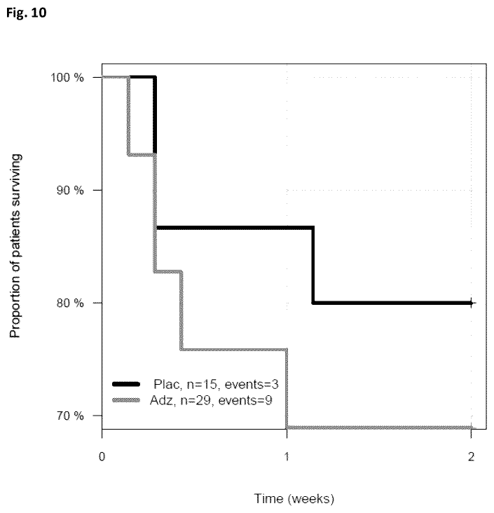

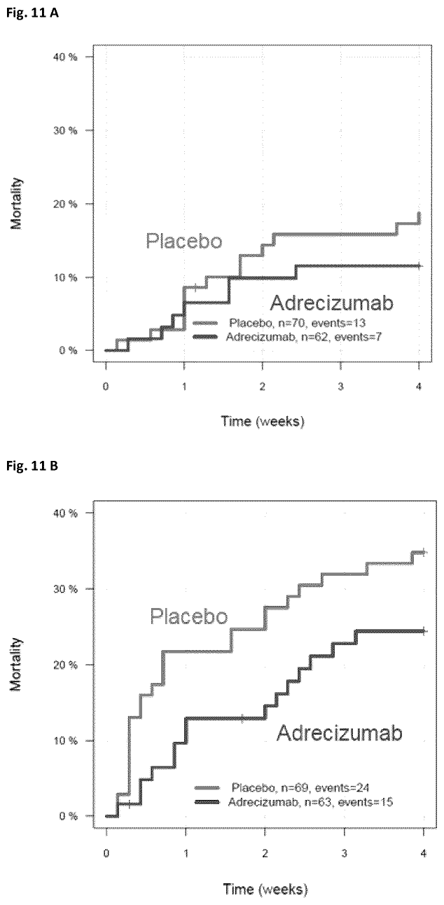

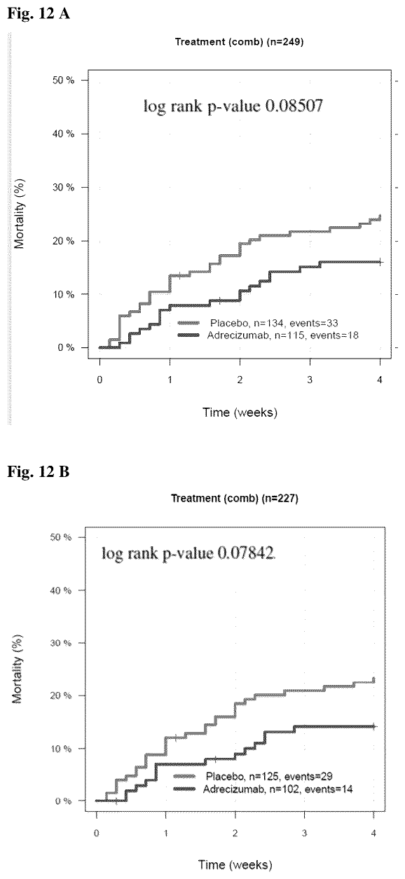

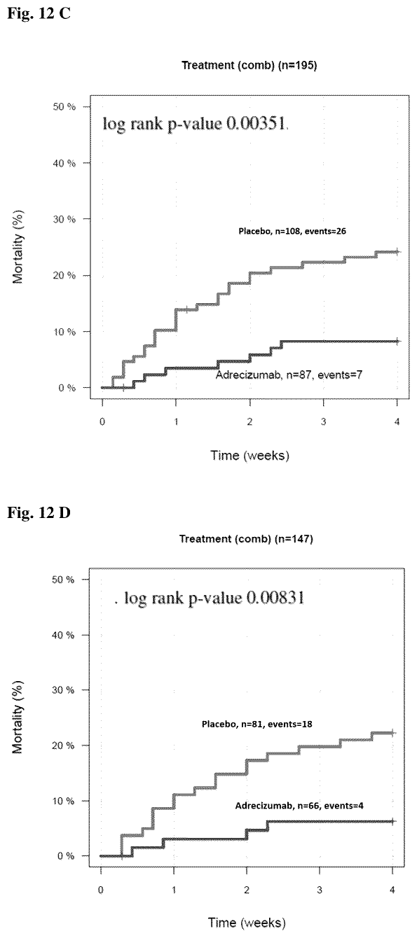

FIG. 2: Dose-response and dose-inhibition curves
a: Dose response curve of human ADM. Maximal cAMP stimulation was adjusted to 100% activation.
b: Dose/inhibition curve of human ADM 22-52 (ADM-receptor antagonist) in the presence of 5.63 nM hADM.
c: Dose/inhibition curve of CT-H in the presence of 5.63 nM hADM.
d: Dose/inhibition curve of MR-H in the presence of 5.63 nM hADM.
e: Dose/inhibition curve of NT-H in the presence of 5.63 nM hADM.
f: Dose response curve of mouse ADM. Maximal cAMP stimulation was adjusted to 100% activation.
g: Dose/inhibition curve of human ADM 22-52 (ADM-receptor antagonist) in the presence of 0.67 nM mADM.
h: Dose/inhibition curve of CT-M in the presence of 0.67 nM mADM.
i: Dose/inhibition curve of MR-M in the presence of 0.67 nM mADM.
j: Dose/inhibition curve of NT-M in the presence of 0.67 nM mADM.
k: Shows the inhibition of ADM by F(ab)2 NT-M and by Fab NT-M.
l: shows the inhibition of ADM by F(ab)2 NT-M and by Fab NT-M.

FIG. 3: This figure shows a typical hADM dose/signal curve. And an hADM dose signal curve in the presence of 100 μg/mL antibody NT-H.

FIG. 4: This figure shows the stability of hADM in human plasma (citrate) in absence and in the presence of NT-H antibody.

FIG. 5: Alignment of the Fab with homologous human framework sequences.

FIG. 6: ADM-concentration in healthy human subjects after NT-H application at different doses up to 60 days.

FIG. 7: Kaplan-Meier survival plots in relation to low (<40.5 ng/mL) and high (≥40.5 ng/mL) DPP3 concentrations. (A) 7-day survival of patients with sepsis in relation to DPP3 plasma concentration; (B) 7-day survival of patients with cardiogenic shock in relation to DPP3 plasma concentrations; (C) 7-day survival of patients with septic shock in relation to DPP3 plasma concentration.

FIG. 8: Kaplan-Meier survival plot for all patients (14-day mortality of patients treated with placebo (Plac) or the N-terminal ADM antibody Adrecizumab (Adz)

FIG. 9: Kaplan-Meier survival plot for patients with DPP3<50 ng/mL (14-day mortality of patients treated with placebo (Plac) or the N-terminal ADM antibody Adrecizumab (Adz)

FIG. 10: Kaplan-Meier survival plot for patients with DPP3 >50 ng/mL (14-day mortality of patients treated with placebo (Plac) or the N-terminal ADM antibody Adrecizumab (Adz)

FIG. 11: Kaplan Meier Plots for the Adrecizumab treatment effect on 28-day mortality in patients with high and low pre-dose bio-ADM concentrations. Investigated populations: PP and PP/DPP3≤70. FIG. 11a shows PP/DPP3≤70—Patients with bio-ADM between 70 and 182 pg/mL (below median and FIG. 11b shows patients with bio-ADM above 182 pg/mL (above median). Calculated HR with 95% CI and log rank p-values (2-sided) are as follows: PP below median: HR=0.630 [0.276-1.44], log rank p=0.270; PP above median: HR=0.870 [0.499-1.52], log rank p=0.634; PP/DPP3≤70 below median: HR=0.607 [0.242-1.52], log rank p=0.284; PP/DPP3≤70 above median: HR=0.623 [0.327-1.19], log rank p=0.151.

FIG. 12: Efficacy of Adrecizumab treatment using different DPP3 threshold values: Kaplan-Meier survival plot (28-day mortality) for patients treated with placebo or the N-terminal ADM antibody Adrecizumab with inclusion of patients (A) with DPP3<50 ng/mL, (B) with DPP3<40 ng/ml, (C) with DPP3<30 ng/ml and (D) with DPP3<22 ng/ml, respectively.

### EXAMPLES

### Example 1—Generation of Antibodies and Determination of their Affinity Constants

Several human and murine antibodies were produced and their affinity constants were determined (see tables 1 and 2). It should be emphasized that the antibodies, antibody fragments and non-Ig scaffolds of the example portion in accordance with the invention are binding to ADM, and thus should be considered as anti-ADM antibodies/antibody fragments/non-Ig scaffolds.

### Peptides/Conjugates for Immunization:

Peptides for immunization were synthesized, see Table 1, (JPT Technologies, Berlin, Germany) with an additional N-terminal Cystein (if no Cystein is present within the selected ADM-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio.

### Mouse Monoclonal Antibody Production:

A Balb/c mouse was immunized with 100 μg Peptide-BSA-Conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intra-venous injection. Splenocytes from the immunized mouse and cells of the myeloma cell line SP2/0 were fused with 1 ml 50% polyethylene glycol for 30s at 37° C. After washing, the cells were seeded in 96-well cell culture plates. Hybrid clones were selected by growing in HAT medium [RPMI 1640 culture medium supplemented with 20% fetal calf serum and HAT-Supplement]. After two weeks the HAT medium is replaced with HT Medium for three passages followed by returning to the normal cell culture medium. The cell culture supernatants were primary screened for antigen specific IgG antibodies three weeks after fusion. The positive tested microcultures were transferred into 24-well plates for propagation. After retesting, the selected cultures were cloned and re-cloned using the limiting-dilution technique and the isotypes were determined (see also Lane, R. D. 1985. J. Immunol. Meth. 81: 223-228; Ziegler et al. 1996. Horm. Metab. Res. 28: 11-1).

Antibodies were produced via standard antibody production methods (Marx et al, 1997. Monoclonal Antibody Production, ATLA 25, 121) and purified via Protein A. The antibody purities were >95% based on SDS gel electrophoresis analysis.

### Human Antibodies:

Human Antibodies were produced by means of phage display according to the following procedure: The human naive antibody gene libraries HAL7/8 were used for the isolation of recombinant single chain F-Variable domains (scFv) against adrenomedullin peptide. The antibody gene libraries were screened with a panning strategy comprising the use of peptides containing a biotin tag linked via two different spacers to the adrenomedullin peptide sequence. A mix of panning rounds using non-specifically bound antigen and streptavidin bound antigen were used to minimize background of non-specific binders. The eluted phages from the third round of panning have been used for the generation of monoclonal scFv expressing E. coli strains. Supernatant from the cultivation of these clonal strains has been directly used for an antigen ELISA testing (see also Hust et al. 2011. Journal of Biotechnology 152, 159-170; Scütte et al. 2009. PLoS One 4, e6625). Positive clones have been selected based on positive ELISA signal for antigen and negative for streptavidin coated micro titer plates. For further characterizations the scFv open reading frame has been cloned into the expression plasmid pOPE107 (Hust et al., J. Biotechn. 2011), captured from the culture supernatant via immobilized metal ion affinity chromatography and purified by a size exclusion chromatography.

Affinity Constants: To determine the affinity of the antibodies to ADM, the kinetics of binding of ADM to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare). (Lorenz et al. 2011. Antimicrob Agents Chemother. 55(1): 165-173).

The monoclonal antibodies were raised against the below depicted ADM regions of human and murine ADM, respectively. The following table represents a selection of obtained antibodies used in further experiments. Selection was based on target region:

TABLE 1
immunization peptides

| | | | | Affinity |
|---|---|---|---|---|
| Sequence | | ADM | | constants |
| Number | Antigen/Immunogen | Region | Designation | Kd (M) |
| SEQ ID: 14 | YRQSMNNFQGLRSFGCRFGTC | 1-21 | NT-H | 5.9 × 10⁻⁹ |
| SEQ ID: 15 | CTVQKLAHQIYQ | 21-32 | MR-H | 2 × 10⁻⁹ |
| SEQ ID: 16 | CAPRSKISPQGY-NH₂ | C-42-52 | CT-H | 1.1 × 10⁻⁹ |
| SEQ ID: 17 | YRQSMNQGSRSNGCRFGTC | 1-19 | NT-M | 3.9 × 10⁻⁹ |
| SEQ ID: 18 | CTFQKLAHQIYQ | 19-31 | MR-M | 4.5 × 10⁻¹⁰ |
| SEQ ID: 19 | CAPRNKISPQGY-NH₂ | C-40-50 | CT-M | 9 × 10⁻⁹ |

The following is a list of further obtained monoclonal antibodies:

TABLE 2

| | | | | max inhibition |
|---|---|---|---|---|
| | | Clone | Affinity | bioassay (%) |
| Target | Source | number | (M) | (see example 2) |
| NT-M | Mouse | ADM/63 | 5.8 × 10⁻⁹ | 45 |
| | Mouse | ADM/364 | 2.2 × 10⁻⁸ | 48 |
| | Mouse | ADM/365 | 3.0 × 10⁻⁸ | |
| | Mouse | ADM/366 | 1.7 × 10⁻⁸ | |
| | Mouse | ADM/367 | 1.3 × 10⁻⁸ | |
| | Mouse | ADM/368 | 1.9 × 10⁻⁸ | |
| | Mouse | ADM/369 | 2.0 × 10⁻⁸ | |
| | Mouse | ADM/370 | 1.6 × 10⁻⁸ | |
| | Mouse | ADM/371 | 2.0 × 10⁻⁸ | |
| | Mouse | ADM/372 | 2.5 × 10⁻⁸ | |
| | Mouse | ADM/373 | 1.8 × 10⁻⁸ | |
| | Mouse | ADM/377 | 1.5 × 10⁻⁸ | |
| | Mouse | ADM/378 | 2.2 × 10⁻⁸ | |
| | Mouse | ADM/379 | 1.6 × 10⁻⁸ | |
| | Mouse | ADM/380 | 1.8 × 10⁻⁸ | |
| | Mouse | ADM/381 | 2.4 × 10⁻⁸ | |
| | Mouse | ADM/382 | 1.6 × 10⁻⁸ | |
| | Mouse | ADM/383 | 1.8 × 10⁻⁸ | |
| | Mouse | ADM/384 | 1.7 × 10⁻⁸ | |
| | Mouse | ADM/385 | 1.7 × 10⁻⁸ | |
| | Mouse | ADM/403 | 1.2 × 10⁻⁸ | |
| | Mouse | ADM/395 | 1.2 × 10⁻⁸ | |
| | Mouse | ADM/396 | 3.0 × 10⁻⁸ | |
| | Mouse | ADM/397 | 1.5 × 10⁻⁸ | |
| MR-M | Mouse | ADM/38 | 4.5 × 10⁻¹⁰ | 68 |
| MR-M | Mouse | ADM/39 | 5.9 × 10⁻⁹ | 72 |
| CT-M | Mouse | ADM/65 | 9.0 × 10⁻⁹ | 100 |
| CT-M | Mouse | ADM/66 | 1.6 × 10⁻⁸ | 100 |
| NT-H | Mouse | ADM/33 | 5.9 × 10⁻⁸ | 38 |
| NT-H | Mouse | ADM/34 | 1.6 × 10⁻⁸ | 22 |
| MR-H | Mouse | ADM/41 | 1.2 × 10⁻⁸ | 67 |
| MR-H | Mouse | ADM/42 | <1 × 10⁻⁸ | |
| MR-H | Mouse | ADM/43 | 2.0 × 10⁻⁹ | 73 |
| MR-H | Mouse | ADM/44 | <1 × 10⁻⁸ | |
| CT-H | Mouse | ADM/15 | <1 × 10⁻⁸ | |
| CT-H | Mouse | ADM/16 | 1.1 × 10⁻⁹ | 100 |
| CT-H | Mouse | ADM/17 | 3.7 × 10⁻⁹ | 100 |
| CT-H | Mouse | ADM/18 | <1 × 10⁻⁸ | |
| hADM | Phage display | ADM/A7 | <1 × 10⁻⁸ | |
| | Phage display | ADM/B7 | <1 × 10⁻⁸ | |
| | Phage display | ADM/C7 | <1 × 10⁻⁸ | |
| | Phage display | ADM/G3 | <1 × 10⁻⁸ | |
| | Phage display | ADM/B6 | <1 × 10⁻⁸ | |
| | Phage display | ADM/B11 | <1 × 10⁻⁸ | |
| | Phage display | ADM/D8 | <1 × 10⁻⁸ | |
| | Phage display | ADM/D11 | <1 × 10⁻⁸ | |
| | Phage display | ADM/G12 | <1 × 10⁻⁸ | |

Generation of antibody fragments by enzymatic digestion: The generation of Fab and F(ab)₂ fragments was done by enzymatic digestion of the murine full-length antibody NT-M. Antibody NT-M was digested using a) the pepsin-based F(ab)₂ Preparation Kit (Pierce 44988) and b) the papain-based Fab Preparation Kit (Pierce 44985). The fragmentation procedures were performed according to the instructions provided by the supplier. Digestion was carried out in case of F(ab)₂-fragmentation for 8 h at 37° C. The Fab-fragmentation digestion was carried out for 16 h, respectively.

Procedure for Fab Generation and Purification: The immobilized papain was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample where added to the spin column tube containing the equilibrated Immobilized Papain. Incubation time of the digestion reaction was done for 16 h on a tabletop rocker at 37° C. The column was centrifuged at 5000×g for 1 minute to separate digest from the Immobilized Papain. Afterwards the resin was washed with 0.5 ml PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 ml of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Coulter and Harris 1983. J. Immunol. Meth. 59, 199-203; Lindner et al. 2010. Cancer Res. 70, 277-87; Kaufmann et al. 2010. PNAS. 107, 18950-5; Chen et al. 2010. PNAS. 107, 14727-32; Uvsal et al. 2009 J. Exp. Med. 206, 449-62; Thomas et al. 2009. J. Exp. Med. 206, 1913-27; Kong et al. 2009 J. Cell Biol. 185, 1275-840).

Procedure for generation and purification of F(ab′)₂ Fragments: The immobilized Pepsin was equilibrated by washing the resin with 0.5 ml of Digestion Buffer and centrifuging the column at 5000×g for 1 minute. The buffer was discarded afterwards. The desalting column was prepared by removing the storage solution and washing it with digestion buffer, centrifuging it each time afterwards at 1000×g for 2 minutes. 0.5 ml of the prepared IgG sample where added to the spin column tube containing the equilibrated Immobilized Pepsin. Incubation time of the digestion reaction was done for 16 h on a tabletop rocker at 37° C. The column was centrifuged at 5000×g for 1 minute to separate digest from the Immobilized Papain. Afterwards the resin was washed with 0.5 mL PBS and centrifuged at 5000×g for 1 minute. The wash fraction was added to the digested antibody that the total sample volume was 1.0 ml. The NAb Protein A Column was equilibrated with PBS and IgG Elution Buffer at room temperature. The column was centrifuged for 1 minute to remove storage solution (contains 0.02% sodium azide) and equilibrated by adding 2 mL of PBS, centrifuge again for 1 minute and the flow-through discarded. The sample was applied to the column and resuspended by inversion. Incubation was done at room temperature with end-over-end mixing for 10 minutes. The column was centrifuged for 1 minute, saving the flow-through with the Fab fragments. (References: Mariani et al. 1991. Mol. Immunol. 28: 69-77; Beale 1987. Exp Comp Immunol 11:287-96; Ellerson et al. 1972. FEBS Letters 24(3):318-22; Kerbel and Elliot 1983. Meth Enzymol 93:113-147; Kulkarni et al. 1985. Cancer Immunol Immunotherapy 19:211-4; Lamovi 1986. Meth Enzymol 121:652-663; Parham et al. 1982. J Immunol Meth 53:133-73; Raychaudhuri et al. 1985. Mol Immunol 22(9):1009-19; Rousseaux et al. 1980. Mol Immunol 17:469-82; Rousseaux et al. 1983. J Immunol Meth 64:141-6; Wilson et al. 1991. J Immunol Meth 138:111-9).

NT-H-Antibody Fragment Humanization: The antibody fragment was humanized by the CDR-grafting method (Jones et al. 1986. Nature 321, 522-525).

The Following Steps where Done to Achieve the Humanized Sequence:

Total RNA extraction: Total RNA was extracted from NT-H hybridomas using the Qiagen kit. First-round RT-PCR: QIAGEN® OneStep RT-PCR Kit (Cat No. 210210) was used. RT-PCR was performed with primer sets specific for the heavy and light chains. For each RNA sample, 12 individual heavy chain and 11 light chain RT-PCR reactions were set up using degenerate forward primer mixtures covering the leader sequences of variable regions. Reverse primers are located in the constant regions of heavy and light chains. No restriction sites were engineered into the primers.

Reaction Setup: 5×QIAGEN® OneStep RT-PCR Buffer 5.0 μl, dNTP Mix (containing 10 mM of each dNTP) 0.8 μl, Primer set 0.5 μl, QIAGEN® OneStep RT-PCR Enzyme Mix 0.8 μl, Template RNA 2.0 μl, RNase-free water to 20.0 μl, Total volume 20.0 μl PCR condition: Reverse transcription: 50° C., 30 min; Initial PCR activation: 95° C., 15 min Cycling: 20 cycles of 94° C., 25 sec; 54° C., 30 sec; 72° C., 30 sec; Final extension: 72° C., 10 min Second-round semi-nested PCR: The RT-PCR products from the first-round reactions were further amplified in the second-round PCR. 12 individual heavy chain and 11 light chain RT-PCR reactions were set up using semi-nested primer sets specific for antibody variable regions.

Reaction Setup: 2×PCR mix 10 μl; Primer set 2 μl; First-round PCR product 8 l; Total volume 20 l; Hybridoma Antibody Cloning Report PCR condition: Initial denaturing of 5 min at 95° C.; 25 cycles of 95° C. for 25 sec, 57° C. for 30 sec, 68° C. for 30 sec; Final extension is 10 min 68° C.

After PCR was finished, PCR reaction samples were run onto agarose gel to visualize DNA fragments amplified. After sequencing more than 15 cloned DNA fragments amplified by nested RT-PCR, several mouse antibody heavy and light chains have been cloned and appear correct. Protein sequence alignment and CDR analysis identifies one heavy chain and one light chain. After alignment with homologous human framework sequences the resulting humanized sequence for the variable heavy chain is the following: see FIG. 5. As the amino acids on positions 26, 40 and 55 in the variable heavy chain and amino acid on position 40 in the variable light are critical to the binding properties, they may be reverted to the murine original. The resulting candidates are depicted below. (Padlan 1991. Mol. Immunol. 28, 489-498; Harris and Bajorath. 1995. Protein Sci. 4, 306-310).

Annotation for the antibody fragment sequences (SEQ ID No.: 6-13; 32 and 33): bold and underlined are the CDR 1, 2, 3 in italic are constant regions; hinge regions are highlighted with bold letters; framework point mutation have a grey letter-background.

(AM-VH-C)
SEQ ID No.: 6
QVQLQQSGAELMKPGASVKISCKATGYTFSRYWIEWVKQRPGHGLEWIGEILPGSGSTNYN
EKFKGKATITADTSSNTAYMQLSSLTSEDSAVYYCTEGYEYDGFDYWGQGTTLTVSSASTKGPSVF
PLAPSSKSTSGGTAALGCLVKDYFPEPVTVSWNSGALTSGVHTFPAVLQSSGLYSLSSVVTVPSSSLGT
QTYICNVNHKPSNTKVDKRVEPK
(AM-VH1)
SEQ ID No.: 7
QVQLVQSGAEVKKPGSSVKVSCKASGYTFSRYWISWVRQAPGQGLEWMGRILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VH2-E40)
SEQ ID No.: 8
QVQLVQSGAEVKKPGSSVKVSCKASGYTFSRYWIEWVRQAPGQGLEWMGRILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VH3-T26-E55)
SEQ ID No.: 9
QVQLVQSGAEVKKPGSSVKVSCKATGYTFSRYWISWVRQAPGQGLEWMGEILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VH4-T26-E40-E55)
SEQ ID No.: 10
QVQLVQSGAEVKKPGSSVKVSCKATGYTESRYWIEWVRQAPGQGLEWMGEILPGSGSTNY
AQKFQGRVTITADESTSTAYMELSSLRSEDTAVYYCTEGYEYDGFDYWGQGTTVTVSSASTKGPSV
FPLAPSSKSTSGGTAALGCLVKDYFPEPVTVSWNSGALTSGVHTFPAVLQSSGLYSLSSVVTVPSSSLG
TQTYICNVNHKPSNTKVDKRVEPK
(AM-VL-C)
SEQ ID No.: 11
DVLLSQTPLSLPVSLGDQATISCRSSQSIVYSNGNTYLEWYLQKPGQSPKLLIYRVSNRFSGVP
DRFSGSGSGTDFTLKISRVEAEDLGVYYCFQGSHIPYTFGGGTKLEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGEC
(AM-VL1)
SEQ ID No.: 12
DVVMTQSPLSLPVTLGQPASISCRSSQSIVYSNGNTYLNWFQQRPGQSPRRLIYRVSNRDSGV
PDRFSGSGSGTDFTLKISRVEAEDVGVYYCFQGSHIPYTFGQGTKLEIKRTVAAPSVFIFPPSD
EQLKSGTASVVCLLNNFYPREAKVQWKVDNALQSGNSQESVTEQDSKDSTYSLSSTLTLSKA
DYEKHKVYACEVTHQGLSSPVTKSFNRGEC
(AM-VL2-E40)
SEQ ID No.: 13
DVVMTQSPLSLPVTLGQPASISCRSSQSIVYSNGNTYLEWFQQRPGQSPRRLIYRVSNRDSGV
PDRFSGSGSGTDFTLKISRVEAEDVGVYYCFQGSHIPYTFGQGTKLEIKRTVAAPSVFIFPPSD
EQLKSGTASVVCLLNNFYPREAKVQWKVDNALQSGNSQESVTEQDSKDSTYSLSSTLTLSKA
DYEKHKVYACEVTHQGLSSPVTKSFNRGEC
(Adrecizumab heavy chain)
SEQ ID NO: 32
QVQLVQSGAEVKKPGSSVKVSCKASGYTFSRYWIEWVRQAPGQGLEWIGEILPGSGSTNYN
QKFQGRVTITADTSTSTAYMELSSLRSEDTAVYYCTEGYEYDGFDYWGQGTTVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDELTKNQVSLTCLVKG
FYPSDIAVEWESNGQPENNYKTTPPVLD
SDGSFFLYSKLTVDKSRWQQGNVFSCSVMHEALHNHYTQKSLSLSPGK
(Adrecizumab light chain)
SEQ ID NO: 33
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKSFNRGEC

### Example 2—Effect of Selected Anti-ADM-Antibodies on Anti-ADM-Bioactivity

The effect of selected ADM-antibodies on ADM-bioactivity was tested in a human recombinant Adrenomedullin receptor cAMP functional assay (Adrenomedullin Bioassay).

Testing of Antibodies Targeting Human or Mouse Adrenomedullin in Human Recombinant Adrenomedullin Receptor cAMP Functional Assay (Adrenomedullin Bioassay)

Materials: Cell line CHO-KI, Receptor Adrenomedullin (CRLR+RAMP3), Receptor Accession Number Cell line: CRLR: U17473; RAMP3: AJ001016

CHO-KI cells expressing human recombinant adrenomedullin receptor (FAST-027C) grown prior to the test in media without antibiotic were detached by gentle flushing with PBS-EDTA (5 mM EDTA), recovered by centrifugation and resuspended in assay buffer (KRH: 5 mM KCl, 1.25 mM MgSO₄, 124 mM NaCl, 25 mM HEPES, 13.3 mM Glucose, 1.25 mM KH₂PO₄, 1.45 mM CaCl₂, 0.5 g/l BSA).

Dose response curves were performed in parallel with the reference agonists (hADM or mADM).

Antagonist test (96 well): For antagonist testing, 6 μl of the reference agonist (human (5.63 nM) or mouse (0.67 nM) adrenomedullin) was mixed with 6 W of the test samples at different antagonist dilutions; or with 6 μl buffer. After incubation for 60 min at room temperature, 12 μl of cells (2,500 cells/well) were added. The plates were incubated for 30 min at room temperature. After addition of the lysis buffer, percentage of DeltaF will be estimated, according to the manufacturer specification, with the HTRF kit from Cis-Bio International (cat n° 62AM2 PEB) hADM 22-52 was used as reference antagonist.

Antibodies Testing cAMP-HTRF Assay

The anti-h-ADM antibodies (NT-H, MR-H, CT-H) were tested for antagonist activity in human recombinant adrenomedullin receptor (FAST-027C) cAMP functional assay in the presence of 5.63 nM Human ADM 1-52, at the following final antibody concentrations: 100 μg/ml, 20 μg/ml, 4 μg/ml, 0.8 μg/ml, 0.16 μg/ml.

The anti-m-ADM antibodies (NT-M, MR-M, CT-M) were tested for antagonist activity in human recombinant ADM receptor (FAST-027C) cAMP functional assay in the presence of 0.67 nM Mouse ADM 1-50, at the following final antibody concentrations: 100 μg/ml, 20 μg/ml, 4 μg/ml, 0.8 μg/ml, 0.16 μg/ml. Data were plotted relative inhibition vs. antagonist concentration (see FIGS. 2 a to 2 l). The maximal inhibition by the individual antibody is given in table 3.

TABLE 3
maximal inhibition of bio-ADM activity

| | Maximal inhibition |
|---|---|
| | of ADM bioactivity |
| Antibody | (ADM-Bioassay) (%) |
| NT-H | 38 |
| MR-H | 73 |
| CT-H | 100 |
| NT-M FAB | 26 |
| NT-M FAB2 | 28 |
| NT-M | 45 |
| MR-M | 66 |
| CT-M | 100 |
| Non specific mouse IgG | 0 |

### Example 3—Stabilization of hADM by the Anti-ADM Antibody

The stabilizing effect of human ADM by human ADM antibodies was tested using a hADM immunoassay.

### Immunoassay for the Quantification of Human Adrenomedullin

The technology used was a sandwich coated tube luminescence immunoassay, based on Acridinium ester labelling.

Labelled compound (tracer): 100 μg (100 μl) CT-H (1 mg/ml in PBS, pH 7.4, AdrenoMed AG Germany) was mixed with 10 μl Acridinium NHS-ester (1 mg/ml in acetonitrile, InVent GmbH, Germany) (EP 0353971) and incubated for 20 min at room temperature. Labelled CT-H was purified by Gel-filtration HPLC on Bio-Sil® SEC 400-5 (Bio-Rad Laboratories, Inc., USA) The purified CT-H was diluted in (300 mmol/L potassium phosphate, 100 mmol/L NaCl, 10 mmol/L Na-EDTA, 5 g/L Bovine Serum Albumin, pH 7.0). The final concentration was approx. 800.000 relative light units (RLU) of labelled compound (approx. 20 ng labeled antibody) per 200 μL. Acridiniumester chemiluminescence was measured by using an AutoLumat LB 953 (Berthold Technologies GmbH & Co. KG).

Solid phase: Polystyrene tubes (Greiner Bio-One International AG, Austria) were coated (18 h at room temperature) with MR-H (AdrenoMed AG, Germany) (1.5 μg MR-H/0.3 mL 100 mmol/L NaCl, 50 mmol/L TRIS/HCl, pH 7.8). After blocking with 5% bovine serum albumin, the tubes were washed with PBS, pH 7.4 and vacuum dried.

Calibration: The assay was calibrated, using dilutions of hADM (BACHEM AG, Switzerland) in 250 mmol/L NaCl, 2 g/L Triton X-100, 50 g/L Bovine Serum Albumin, 20 tabs/L Protease Inhibitor Cocktail (Roche Diagnostics AG, Switzerland).

hADM Immunoassay: 50 μl of sample (or calibrator) was pipetted into coated tubes, after adding labeled CT-H (200 μl), the tubes were incubated for 4 h at 4° C. Unbound tracer was removed by washing 5 times (each 1 ml) with washing solution (20 mM PBS, pH 7.4, 0.1% Triton X-100).

Tube-bound chemiluminescence was measured by using the LB 953: FIG. 3 shows a typical hADM dose/signal curve. And an hADM dose signal curve in the presence of 100 μg/mL antibody NT-H. NT-H did not affect the described hADM immunoassay.

Stability of human Adrenomedullin: Human ADM was diluted in human Citrate plasma (final concentration 10 nM) and incubated at 24° C. At selected time points, the degradation of hADM was stopped by freezing at −20° C. The incubation was performed in absence and presence of NT-H (100 μg/ml). The remaining hADM was quantified by using the hADM immunoassay described above.

FIG. 4 shows the stability of hADM in human plasma (citrate) in absence and in the presence of NT-H antibody. The half-life of hADM alone was 7.8 h and in the presence of NT-H, the half-life was 18.3 h. (2.3 times higher stability).

### Example 4—Sepsis Mortality

### a) Early Treatment of Sepsis

Animal model: 12-15 week-old male C57l/6 mice (Charles River Laboratories, Germany) were used for the study. Peritonitis had been surgically induced under light isofluran anesthesia. Incisions were made into the left upper quadrant of the peritoneal cavity (normal location of the cecum). The cecum was exposed and a tight ligature was placed around the cecum with sutures distal to the insertion of the small bowel. One puncture wound was made with a 24-gauge needle into the cecum and small amounts of cecal contents were expressed through the wound. The cecum was replaced into the peritoneal cavity and the laparotomy site was closed. Finally, animals were returned to their cages with free access to food and water. 500 μl saline were given s.c. as fluid replacement.

Application and dosage of the compound (NT-M, MR-M, CT-M): Mice were treated immediately after CLP (early treatment). CLP is the abbreviation for cecal ligation and puncture (CLP).

Study groups: Three compounds were tested versus: vehicle and versus control compound treatment. Each group contained 5 mice for blood drawing after 1 day for BUN (serum blood urea nitrogen test) determination. Ten further mice per each group were followed over a period of 4 days.

Group Treatment (10l/g bodyweight) dose/Follow-Up:
1 NT-M, 0.2 mg/ml survival over 4 days
2 MR-M, 0.2 mg/ml survival over 4 days
3 CT-M, 0.2 mg/ml survival over 4 days
4 non-specific mouse IgG, 0.2 mg/ml survival over 4 days
5 control—PBS 10 μl/g bodyweight survival over 4 days

Clinical chemistry: Blood urea nitrogen (BUN) concentrations for renal function were measured baseline and day 1 after CLP. Blood samples were obtained from the cavernous sinus with a capillary under light ether anaesthesia. Measurements were performed by using an AU 400 Olympus Multianalyser. The 4-day mortality and the average BUN concentrations are given in table 4.

TABLE 4
4-day mortality and BUN concentrations

| | survival | BUN pre CLP | BUN day |
|---|---|---|---|
| 4-day mortality | (%) | (mM) | 1 (mM) |
| PBS | 0 | 8.0 | 23.2 |
| non-specific mouse IgG | 0 | 7.9 | 15.5 |
| CT-M | 10 | 7.8 | 13.5 |
| MR-M | 30 | 8.1 | 24.9 |
| NT-M | 70 | 8.8 | 8.2 |

It can be seen from Table 4 that the NT-M antibody reduced mortality considerably. After 4 days 70% of the mice survived when treated with NT-M antibody. When treated with MR-M antibody 30% of the animals survived and when treated with CT-M antibody 10% of the animals survived after 4 days. In contrast thereto all mice were dead after 4 days when treated with unspecific mouse IgG. The same result was obtained in the control group where PBS (phosphate buffered saline) was administered to mice. The blood urea nitrogen or BUN test is used to evaluate kidney function, to help diagnose kidney disease, and to monitor patients with acute or chronic kidney dysfunction or failure. The results of the S-BUN Test revealed that the NT-M antibody was the most effective to protect the kidney.

### b) Late Treatment of Sepsis

Animal model: 12-15 week-old male C57Bl/6 mice (Charles River Laboratories, Germany) were used for the study. Peritonitis had been surgically induced under light isofluran anesthesia. Incisions were made into the left upper quadrant of the peritoneal cavity (normal location of the cecum). The cecum was exposed and a tight ligature was placed around the cecum with sutures distal to the insertion of the small bowel. One puncture wound was made with a 24-gauge needle into the cecum and small amounts of cecal contents were expressed through the wound. The cecum was replaced into the peritoneal cavity and the laparotomy site was closed. Finally, animals were returned to their cages with free access to food and water. 500 μl saline were given s.c. as fluid replacement.

Application and dosage of the compound (NT-M FAB2): NT-M FAB2 was tested versus: vehicle and versus control compound treatment. Treatment was performed after full development of sepsis, 6 hours after CLP (late treatment). Each group contained 4 mice and were followed over a period of 4 days.

Group Treatment (10 μl/g bodyweight) dose/Follow-Up:
1 NT-M, FAB2 0.2 mg/ml survival over 4 days
2 control non-specific mouse IgG, 0.2 mg/ml survival over 4 days
3 vehicle:—PBS 101/g bodyweight survival over 4 days

TABLE 5
4-day mortality

| 4 day mortality | survival (%) |
|---|---|
| PBS | 0 |
| Non-specific mouse IgG | 0 |
| NT-M FAB2 | 75 |

It can be seen from Table 5 that the NT-M FAB 2 antibody reduced mortality considerably. After 4 days 75% of the mice survived when treated with NT-M FAB 2 antibody. In contrast thereto all mice were dead after 4 days when treated with non-specific mouse IgG. The same result was obtained in the control group where PBS (phosphate buffered saline) was administered to mice.

### Example 5—Administration of NT-H in Healthy Humans

The study was conducted in healthy male subjects as a randomized, double-blind, placebo-controlled, study with single escalating doses of NT-H antibody administered as intravenous (i.v.) infusion in 3 sequential groups of 8 healthy male subjects each (1st group 0.5 mg/kg, 2nd group 2 mg/kg, 3rd group 8 mg/kg) of healthy male subjects (n=6 active, n=2 placebo for each group). The main inclusion criteria were written informed consent, age 18-35 years, agreement to use a reliable way of contraception and a BMI between 18 and 30 kg/m². Subjects received a single i.v. dose of NT-H antibody (0.5 mg/kg; 2 mg/kg; 8 mg/kg) or placebo by slow infusion over a 1-hour period in a research unit. The baseline ADM-values in the 4 groups did not differ. Median ADM values were 7.1 pg/mL in the placebo group, 6.8 pg/mL in the first treatment group (0.5 mg/kg), 5.5 pg/mL in second treatment group (2 mg/kg) and 7.1 pg/mL in the third treatment group (8 mg/mL). The results show, that ADM-values rapidly increased within the first 1.5 hours after administration of NT-H antibody in healthy human individuals, then reached a plateau and slowly declined (FIG. 6).

### Example 6—Methods for the Measurement of DPP3 Protein and DPP3 Activity

Generation of antibodies and determination DPP3 binding ability: Several murine antibodies were produced and screened by their ability of binding human DPP3 in a specific binding assay (see Table 6).

Peptides/conjugates for immunization: DPP3 peptides for immunization were synthesized, see Table 6, (JPT Technologies, Berlin, Germany) with an additional N-terminal cystein (if no cystein is present within the selected DPP3-sequence) residue for conjugation of the peptides to Bovine Serum Albumin (BSA). The peptides were covalently linked to BSA by using Sulfolink-coupling gel (Perbio-science, Bonn, Germany). The coupling procedure was performed according to the manual of Perbio. Recombinant GST-hDPP3 was produced by USBio (United States Biological, Salem, MA, USA).

Immunization of mice, immune cell fusion and screening: Balb/c mice were intraperitoneally (i.p.) injected with 84 μg GST-hDPP3 or 100 μg DPP3-peptide-BSA-conjugates at day 0 (emulsified in TiterMax Gold Adjuvant), 84 μg or 100 μg at day 14 (emulsified in complete Freund's adjuvant) and 42 μg or 50 μg at day 21 and 28 (in incomplete Freund's adjuvant). At day 49 the animal received an intravenous (i.v.) injection of 42 μg GST-hDPP3 or 50 μg DPP3-peptide-BSA-conjugates dissolved in saline. Three days later the mice were sacrificed and the immune cell fusion was performed.

Splenocytes from the immunized mice and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium [RPMI 1640 culture medium supplemented with 20% fetal calf serum and HAT-Supplement]. After one week, the HAT medium was replaced with HT Medium for three passages followed by returning to the normal cell culture medium. The cell culture supernatants were primarily screened for recombinant DPP3 binding IgG antibodies two weeks after fusion. Therefore, recombinant GST-tagged hDPP3 (USBiologicals, Salem, USA) was immobilized in 96-well plates (100 ng/well) and incubated with 50 μl cell culture supernatant per well for 2 hours at room temperature. After washing of the plate, 50 μl/well POD-rabbit anti mouse IgG was added and incubated for 1 h at RT. After a next washing step, 50 μl of a chromogen solution (3.7 mM o-phenylen-diamine in citrate/hydrogen phosphate buffer, 0.012% H₂O₂) were added to each well, incubated for 15 minutes at RT and the chromogenic reaction stopped by the addition of 50 μl 4N sulfuric acid. Absorption was detected at 490 mm.

The positive tested microcultures were transferred into 24-well plates for propagation. After retesting the selected cultures were cloned and re-cloned using the limiting-dilution technique and the isotypes were determined.

### Mouse Monoclonal Antibody Production

Antibodies raised against GST-tagged human DPP3 or DPP3-peptides were produced via standard antibody production methods (Marx et al. 1997) and purified via Protein A. The antibody purities were ≥90% based on SDS gel electrophoresis analysis.

Characterization of Antibodies—Binding to hDPP3 and/or Immunization Peptide

To analyze the capability of DPP3/immunization peptide binding by the different antibodies and antibody clones a binding assay was performed:

Solid phase: Recombinant GST-tagged hDPP3 (SEQ ID NO. 34) or a DPP3 peptide (immunization peptide, SEQ ID NO. 35) was immobilized onto a high binding microtiter plate surface (96-Well polystyrene microplates, Greiner Bio-One international AG, Austria, 1 μg/well in coupling buffer [50 mM Tris, 100 mM NaCl, pH7.8], 1 h at RT). After blocking with 5% bovine serum albumin, the microplates were vacuum dried.

Labelling procedure (tracer): 100 μg (100 μl) of the different antiDPP3 antibodies (detection antibody, 1 mg/ml in PBS, pH 7.4) were mixed with 10 μl acridinium NHS-ester (1 mg/ml in acetonitrile, InVent GmbH, Germany; EP 0 353 971) and incubated for 30 min at room temperature. Labelled antiDPP3 antibody was purified by gel-filtration HPLC on Shodex Protein 5 μm KW-803 (Showa Denko, Japan). The purified labelled antibody was diluted in assay buffer (50 mmol/1 potassium phosphate, 100 mmol/1 NaCl, 10 mmol/l Na₂-EDTA, 5 g/l bovine serum albumin, 1 g/l murine IgG, 1 g/l bovine IgG, 50 μmol/l amastatin, 100 μmol/l leupeptin, pH 7.4). The final concentration was approx. 5-7*10⁶ relative light units (RLU) of labelled compound (approx. 20 ng labelled antibody) per 200 μl. acridinium ester chemiluminescence was measured by using a Centro LB 960 luminometer (Berthold Technologies GmbH & Co. KG).

hDPP3 binding assay: the plates were filled with 200 μl of labelled and diluted detection antibody (tracer) and incubated for 2-4 h at 2-8° C. Unbound tracer was removed by washing 4 times with 350 μl washing solution (20 mM PBS, pH 7.4, 0.1% Triton X-100). Well-bound chemiluminescence was measured by using the Centro LB 960 luminometer (Berthold Technologies GmbH & Co. KG).

Characterization of Antibodies—hDPP3-Inhibition Analysis

To analyze the capability of DPP3 inhibition by the different antibodies and antibody clones a DPP3 activity assay with known procedure (Jones et al., 1982) was performed. Recombinant GST-tagged hDPP3 was diluted in assay buffer (25 ng/ml GST-DPP3 in 50 mM Tris-HCl, pH7.5 and 100 μM ZnCl₂) and 200 μl of this solution incubated with 10 μg of the respective antibody at room temperature. After 1 hour of pre-incubation, fluorogenic substrate Arg-Arg-βNA (20 μl, 2 mM) was added to the solution and the generation of free PNA over time was monitored using the Twinkle LB 970 microplate fluorometer (Berthold Technologies GmbH & Co. KG) at 37° C. Fluorescence of PNA is detected by exciting at 340 nm and measuring emission at 410 nm. Slopes (in RFU/min) of increasing fluorescence of the different samples are calculated. The slope of GST-hDPP3 with buffer control is appointed as 100% activity. The inhibitory ability of a possible capture-binder is defined as the decrease of GST-hDPP3 activity by incubation with said capture-binder in percent.

The following table represents a selection of obtained antibodies and their binding rate in Relative Light Units (RLU) as well as their relative inhibitory ability (%; table 6). The monoclonal antibodies raised against the below depicted DPP3 regions, were selected by their ability to bind recombinant DPP3 and/or immunization peptide, as well as by their inhibitory potential.

All antibodies raised against the GST-tagged, full-length form of recombinant hDPP3 show a strong binding to immobilized GST-tagged hDPP3. Antibodies raised against the SEQ ID NO.: 35 peptide bind as well to GST-hDPP3. The SEQ ID NO.: 35 antibodies also strongly bind to the immunization peptide.

TABLE 6
list of antibodies raised against full-length or sequences of hDPP3 and their ability to
bind hDPP3 (SEQ ID NO.: 34) or immunization peptide (SEQ ID NO.: 35) in RLU, as well as
the maximum inhibition of recombinant GST-hDPP3.

| | | | | hDPP3 | immunization | Max. |
|---|---|---|---|---|---|---|
| Sequence | | hDPP3 | | binding | peptide | inhibition |
| number | Antigen/Immunogen | region | Clone | [RLU] | binding [RLU] | of hDPP3 |
| SEQ ID | GST tagged recombinant | 1-737 | 2552 | 3.053.621 | 0 | 65% |
| NO.: 34 | FL-hDPP3 | | 2553 | 3.777.985 | 0 | 35% |
| | | | 2554 | 1.733.815 | 0 | 30% |
| | | | 2555 | 3.805.363 | 0 | 25% |
| SEQ ID | CETVINPETGEQIQSWYRSGE | 474-493 | 1963 | 141.822 | 2.163.038 | 60% |
| NO.: 35 | | | 1964 | 100.802 | 2.041.928 | 60% |
| | | | 1965 | 99.493 | 1.986.794 | 70% |
| | | | 1966 | 118.097 | 1.990.702 | 65% |
| | | | 1967 | 113.736 | 1.909.954 | 70% |
| | | | 1968 | 105.696 | 2.017.731 | 65% |
| | | | 1969 | 82.558 | 2.224.025 | 70% |

The development of a luminescence immunoassay for the quantification of DPP3 protein concentrations (DPP3-LIA) as well as an enzyme capture activity assay for the quantification of DPP3 activity (DPP3-ECA) have been described recently (Rehfeld et al. 2019. JALM 3(6): 943-953), which is incorporated here in its entirety by reference.

### Example 7—DPP3 in Septic and Cardiogenic Shock

DPP3 concentration in plasma of patients with sepsis/septic shock and cardiogenic shock was determined and related to the short term-mortality of the patients.

### a) Study Cohort—Sepsis/Septic Shock

In 574 plasma samples from patients of the Adrenomedullin and Outcome in Severe Sepsis and Septic Shock (AdrenOSS-1) study DPP3 was measured. AdrenOSS-1 is a prospective, observational, multinational study including 583 patients admitted to the intensive care unit with sepsis or septic shock (Hollinger et al., 2018 Aug. 22; 3(6): 1424-1433). 292 patients were diagnosed with septic shock.

### b) Study Cohort—Cardiogenic Shock

Plasma samples from 108 patients that were diagnosed with cardiogenic shock were screened for DPP3. Blood was drawn within 6 h from detection of cardiogenic shock. Mortality was followed for 7 days.

hDPP3 immunoassay: An immunoassay (LIA) or an enzyme activity assay (ECA) detecting the amount of human DPP3 (LIA) or the activity of human DPP3 (ECA), respectively, was used for determining the DPP3 level in patient plasma. Antibody immobilization, labelling and incubation were performed as described in Rehfeld et al. (Rehfeld et al. 2019. JALM 3(6): 943-953).

Results: Short-term patients' survival in sepsis patients was related to the DPP3 plasma concentration at admission. Patients with DPP3 plasma concentration above 40.5 ng/mL (3rd Quartile) had an increased mortality risk compared to patients with DPP3 plasma concentrations below this threshold (FIG. 7A). Applying this cut-off to the sub-cohort of septic shock patients, revealed an even more pronounced risk for short-term mortality in relation to high DPP3 plasma concentrations (FIG. 7B). When the same cut-off is applied to patients with cardiogenic shock, also an increased risk for short-term mortality within 7 days is observed in patients with high DPP3 (FIG. 7C).

### Example 8—NT-ADM Antibodies in Patients with Septic Shock (AdrenOSS-2)

AdrenOSS-2 is a double-blind, placebo-controlled, randomized, multicenter, proof of concept and dose-finding phase II clinical trial to investigate the safety, tolerability and efficacy of the N-terminal ADM antibody named Adrecizumab in patients with septic shock and elevated adrenomedullin (Geven et al. BMJ Open 2019; 9:e024475). In total, 301 patients with septic shock and bio-ADM concentration >70 pg/mL were randomized (2:1:1) to treatment with a single intravenous infusion over approximately 1 hour with either placebo (n=152), adrecizumab 2 ng/kg (n=72) or adrecizumab 4 ng/kg (n=77). All-cause mortality within 28 (90) days after inclusion was 25.8% (34.8%). Mean age was 68.4 years and 61% were male. For the per protocol analysis, n=294 patients remained eligible, and 14-day all-cause mortality rate was 18.5%.

In patients treated with Adrecizumab (both doses combined, per protocol population), a trend to lower short-term mortality (14 days post admission) was observed compared to placebo (Hazard ratio (HR) 0.701 [0.408-1.21], p=0.100) (FIG. 8). Surprisingly, in patients with a DPP3 concentration on admission below 50 ng/mL, the treatment effect was more pronounced (n=244, HR 0.426, p=0.007) (FIG. 9), while in patients with an elevated DPP3 (above 50 ng/mL, n=44), outcome was comparable between Adrecizumab and placebo (HR 1.69, p=0.209) (FIG. 10).

Treatment effects for different DPP3 thresholds (14-day mortality) are summarized in table 7.

TABLE 7
Hazard risks (HR) for 14-day mortality with
different pre-dose DPP3 concentrations

| | | | p-value |
|---|---|---|---|
| | n | HR | (1-sided, log rank) |
| all | 294 | 0.701 | 0.100 |
| DPP3 <90 | 273 | 0.606 | 0.049 |
| DPP3 <70 | 261 | 0.546 | 0.027 |
| DPP3 <60 | 254 | 0.449 | 0.008 |
| DPP3 <50 | 244 | 0.426 | 0.007 |
| DPP3 <40 | 227 | 0.385 | 0.005 |

### Example 9: Treatment Effects in Patients with Bio-ADM Below 70 μg/mL

To show that the anti-ADM treatment works also in patients with bio-ADM levels lower than 70 μg/mL, it was investigated in the AdrenOSS-2 study cohort, whether in the included patient population (all with bio-ADM >70 μg/mL) patients would exhibit a different treatment effect depending on their pre-dose bio-ADM concentration. No such interaction was detected which in other words means, that the beneficial treatment effect was detectable for all patients independent of their pre-dose bio-ADM concentration. This finding strongly suggests, that treatment works also in patients with bio-ADM levels lower than 70 μg/mL, with the likely (and acceptable) restriction that it such effect might not be expected in patients with bio-ADM concentrations in the healthy normal range.

P-value for interaction term between treatment and pre-dose bio-ADM:
Per Protocol population: p=0.279
Per Protocol population/with pre-dose DPP3<70 ng/mL: p=0.150

This is further illustrated in Kaplan Meier Plots, where the patient populations were separated by their pre-dose bio-ADM concentration (below/above bio-ADM median) (see FIG. 11a and FIG. 11b).

### Example 10—Low DPP3 Thresholds in Sepsis and Septic Shock (AdrenOSS-1)

The AdrenOSS-1 study as described in example 7 was used to analyze, whether patients with DPP3 levels well below 50 ng/ml at baseline show an increase in DPP3 plasma concentration above 50 ng/ml in the following days.

### Results:

The DPP3 plasma levels in septic shock patients (n=292) at baseline (day 1, DPP3.d1) were statistically analysed with the aim of determining a threshold were patients show an increase in DPP3 plasma concentration above 50 ng/ml on the following days. The DPP3 concentration of 50 ng/ml reflects a threshold above which patients a) are beyond the DPP3 upper limit of normal, b) have a high organ dysfunction and mortality rate (Blet et al. 2021. Crit Care 25(1): 61) and c) have been shown to have a lower treatment effect for the N-terminal ADM-antibody Adrecizumab (WO2021/170838).

TABLE 8
DPP3 plasma levels in septic shock patients

| DPP3 concentration thresholds | DPP3.d1 < 50 | DPP3.d1 < 40 | DPP3.d1 < 30 | DPP3.d1 < 22 |
|---|---|---|---|---|
| at baseline (d 1) | ng/ml | ng/ml | ng/ml | ng/ml |
| DPP3 below 50 ng/ml during | 201 | 187 | 149 | 104 |
| ICU stay | | | | |
| rising DPP3 above 50 ng/ml | 22 | 14 | 7 | 1 |
| during ICU stay | | | | |
| Total number of patients | 223 | 201 | 156 | 105 |
| % of patients with rising DPP3 | 10% | 7% | 4% | 1% |
| above 50 ng/ml during ICU stay | | | | |
| on days 2 and 3 | | | | |

Different DPP3 threshold values at baseline (d1) were analysed for the percentage of patients with rising DPP3 plasma concentrations above 50 ng/ml in the following days (day 2 and day 3). Table 8 shows that the lower the DPP3 plasma concentration at baseline (DPP3.d1), the lower the percentage of patients with an increase in DPP3 above 50 ng/ml in the following days. In this septic shock population, 223 and 156 patients have a DPP3 concentration below <50 or <30 ng/ml at baseline, respectively. For 156 patients below 30 ng/ml at baseline, 7 (4%) patients show a DPP3 concentration rise above the DPP3 threshold of 50 ng/ml in the following days. On the other hand, among the 67 septic shock patients with DPP3 plasma concentrations between 30 and 50 ng/ml at baseline, 15 (22.4%) patients increased their DPP3 plasma levels above 50 ng/ml in the following days. As a consequence, a low DPP3 threshold (well below 50 ng/ml) is suitable to show, that DPP3 levels will increase during follow-up. As it was shown that patients with DPP3 concentrations above 50 ng/ml will have a significantly reduced efficacy for treatment with an anti-ADM antibody (Adrecizumab), these results suggest, that lower threshold levels for DPP3 may be used for a treatment decision for anti-ADM antibodies (Adrecizumab) therapy at baseline in patients with septic shock. The low DPP3 concentration threshold at baseline (d1) ensures, that the DPP3 pathological pathway (which is associated with high short-term organ dysfunction and mortality) is not the predominant pathway in the selected septic shock population. Therefore, this septic shock population with DPP3 concentrations below the above-mentioned threshold ranges at baseline may have a higher treatment effect from, e.g., anti-ADM antibody therapy (Adrecizumab).

In a second step, the septic shock population with bio-ADM plasma concentrations above 70 μg/ml were analyzed. Bio-ADM concentrations above 70 μg/ml have been associated with sepsis severity, development of organ dysfunction, including vasopressor/inotrope dependency (Marino et al. 2014. Critical Care 18: R34; Caironi et al. 2017. Chest 152(2):312-320; Mebazaa et al. 2018. Crit Care 22: 354). Different DPP3 threshold values at baseline (dl) were analysed for the percentage of patients with rising DPP3 plasma concentrations above 50 ng/ml in the following days (day 2 and day 3). Table 9 again shows that the lower the DPP3 plasma concentration at baseline (DPP3.d1), the lower the percentage of septic shock patients with high bio-ADM with an increase in DPP3 above 50 ng/ml in the following days. In this septic shock and high bio-ADM population, 154 and 100 patients have a DPP3 concentration below <50 or <30 ng/ml at baseline, respectively. For 100 patients below 30 ng/ml at baseline, 4 (4%) patients show a DPP3 concentration rise above the DPP3 threshold of 50 ng/ml in the following 2 days. On the other hand, among the 54 septic shock patients with DPP3 plasma concentrations between 30 and 50 ng/ml at baseline, 13 (24.1%) patients increased their DPP3 plasma levels above 50 ng/ml in the following days.

TABLE 9
DPP3 plasma levels in septic shock patients with a bio-ADM level above 70 pg/ml

| DPP3 concentration cut | DPP3.d1 < 50 | DPP3.d1 < 40 | DPP3.d1 < 30 | DPP3.d1 < 22 |
|---|---|---|---|---|
| offs at baseline (d 1) | ng/ml | ng/ml | ng/ml | ng/ml |
| DPP3 below 50 ng/ml during | 137 | 125 | 96 | 61 |
| ICU stay | | | | |
| rising DPP3 above 50 ng/ml | 17 | 10 | 4 | 0 |
| during ICU stay | | | | |
| Total number of patients | 154 | 135 | 100 | 61 |
| % of patients with rising DPP3 | 11% | 7% | 4% | 0% |
| above 50 ng/ml during ICU | | | | |
| stay on days 2 and 3 | | | | |

### Example 11—Lower DPP3 Thresholds in AdrenOSS-2 and Efficacy of NT-ADM Antibody Therapy

To validate the findings from example 10 and for proof of concept, the different lower thresholds for DPP3 at baseline (day 1) were assessed for efficacy of the anti-ADM antibody (Adrecizumab) therapy in the septic shock population with high bio-ADM from the AdrenOSS-2 study cohort and patients with a DPP3 plasma value above the different lower thresholds were excluded from the analyses. Efficacy of the anti-bio-ADM antibody therapy was specifically assessed to what concerns the mortality endpoint in the placebo and treated arms.

### Results:

The DPP3 plasma levels in septic shock with high bio-ADM (above 70 μg/ml) patients (n=298) at baseline and in the following 144 h were statistically analysed with the aim of determining a DPP3 threshold at baseline to assess anti-ADM antibody therapy efficacy. Patients above the respective DPP3 plasma thresholds were excluded from the analyses. Different DPP3 plasma concentration thresholds have been applied for 28-day all-cause mortality evaluation using Kaplan-Meier plots comparing anti-ADM antibody therapy vs. placebo. The log-rank test was chosen for showing differences in mortality rates among treatment groups. Hazard ratios (HR) were calculated for each DPP3 plasma concentration threshold to estimate the reduction in mortality risk imposed by anti-ADM antibody therapy relative to placebo.

The DPP3 plasma concentration thresholds at baseline used were 50 ng/ml, 40 ng/ml, 30 ng/ml and 22 ng/ml, respectively. For each threshold, the number of patients excluded from all cause mortality analysis was determined. For the 50, 40, 30 and 22 ng/ml thresholds, 16%, 24%, 35% and 51% of patients were excluded from the analyses, respectively.

The HRs for each DPP3 plasma concentration threshold at baseline to estimate the reduction in mortality by the anti-ADM antibody therapy relative to placebo were also determined. For the 50, 40, 30 and 22 ng/ml thresholds, HRs were 0.606, 0.568, 0.309 and 0.258, respectively. This analysis shows that the lower the DPP3 plasma concentration threshold, the higher the reduction in mortality in the treated group. Similarly, all-cause mortality analysis using Kaplan-Meier indicate that the lower the DPP3 plasma concentration threshold, the more pronounced and significant is the reduction in mortality in the treated arm (FIG. 12 A-D).

The percentage of patients that show an increase in DPP3 plasma concentration above 50 ng/ml in the following 144 h was also estimated for each DPP3 plasma concentration threshold at baseline. Similar to the results from the AdrenOSS-1 study in example 10 it was shown that: the lower the DPP3 plasma concentration threshold at baseline, the lower the percentage of patients that showed an increase in their DPP3 plasma levels above the 50 ng/ml threshold in the following days. In this septic shock and high bio-ADM population, 249 and 195 patients have a DPP3 concentration below <50 or <30 ng/ml at baseline, respectively. For 195 patients below 30 ng/ml at baseline, 16 (8%) patients show a DPP3 concentration rise above the DPP3 threshold of 50 ng/ml in the following 6 days. On the other hand, among the 54 septic shock patients with DPP3 plasma concentrations between 30 and 50 ng/ml at baseline, 11 (20.4%) patients increased their DPP3 plasma levels above 50 ng/ml in the following days. These results show, that a lower DPP3 threshold (well below 50 ng/ml) is suitable to guide the use of and select the patients prone to benefit from anti-ADM antibody therapy.

The different thresholds for DPP3 levels at baseline (day 1) were further used for subgroup analyses for 28-day all-cause mortality evaluation using Kaplan-Meier plots in the treated and placebo arms comparing anti-ADM antibody therapy (Adrecizumab) vs. placebo. The DPP3 plasma concentration thresholds at baseline used were 50 ng/ml and 30 ng/ml.

When assessing all-cause mortality in the septic shock population with DPP3 levels below the threshold of 30 ng/ml (n=195), the mortality in the anti-ADM antibody (Adrecizumab) therapy arm was surprisingly significantly reduced compared to the placebo arm (FIG. 12 C). The same results are observed when including only patients with DPP3 values below 50 ng/ml, the mortality in the treated arm is lower than the placebo arm. Finally, when assessing all-cause mortality from admission to 28 days in septic shock patients with DPP3 levels below 50 ng/ml at baseline and continuously low (<50 ng/ml) DPP3 levels during the next 144h, the mortality rate in the treated arm is significantly lower than in the placebo arm (FIG. 13). As a consequence, a low DPP3 threshold (well below 50 ng/ml) at baseline is most suitable to select patients that will benefit from the anti-ADM antibody therapy.

In summary, patients with DPP3 levels above the threshold of 30 ng/ml at baseline have a higher chance to show an increase in DPP3 plasma concentration in the following days. The following increase in DPP3 plasma concentration above 50 ng/ml is associated with a lower efficacy of anti-ADM antibody therapy. Therefore, to stratify patients for anti-ADM antibody therapy, lower thresholds, well below 50 ng/ml, preferably ≤40 ng/ml or in the range between 22 and 40 ng/ml, most preferred at a threshold of 30 ng/ml should be used.

SEQUENCES

SEQ ID No.: 1
GYTFSRYW
SEQ ID No.: 2
ILPGSGST
SEQ ID No.: 3
TEGYEYDGFDY
SEQ ID No.: 4
QSIVYSNGNTY
SEQUENCE “RVS” (not part of the Sequencing Listing):
RVS
SEQ ID No.: 5
FQGSHIPYT
SEQ ID No.: 6 (AM-VH-C)
QVQLQQSGAELMKPGASVKISCKATGYTFSRYWIEWVKQRPGHGLEWIGEILPGSGSTNYNE
KFKGKATITADTSSNTAYMQLSSLTSEDSAVYYCTEGYEYDGFDYWGQGTTLTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKRVEPK
SEQ ID No.: 7 (AM-VH1)
QVQLVQSGAEVKKPGSSVKVSCKASGYTFSRYWISWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 8 (AM-VH2-E40)
QVQLVQSGAEVKKPGSSVKVSCKASGYTFSRYWIEWVRQAPGQGLEWMGRILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 9 (AM-VH3-T26-E55)
QVQLVQSGAEVKKPGSSVKVSCKATGYTFSRYWISWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 10 (AM-VH4-T26-E40-E55)
QVQLVQSGAEVKKPGSSVKVSCKATGYTFSRYWIEWVRQAPGQGLEWMGEILPGSGSTNYA
QKFQGRVTITADESTSTAYMELSSLRSEDTAVYYCTEGYEYDGFDYWGQGTTVTVSSASTKG
PSVFPLAPSSKSTSGGTAALGCLVKDYFPEPVTVSWNSGALTSGVHTFPAVLQSSGLYSLSSV
VTVPSSSLGTQTYICNVNHKPSNTKVDKRVEPK
SEQ ID No.: 11 (AM-VL-C)
DVLLSQTPLSLPVSLGDQATISCRSSQSIVYSNGNTYLEWYLQKPGQSPKLLIYRVSNRFSGVP
DRFSGSGSGTDFTLKISRVEAEDLGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No.: 12 (AM-VL1)
DVVMTQSPLSLPVTLGQPASISCRSSQSIVYSNGNTYLNWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No.: 13 (AM-VL2-E40)
DVVMTQSPLSLPVTLGQPASISCRSSQSIVYSNGNTYLEWFQQRPGQSPRRLIYRVSNRDSGVP
DRFSGSGSGTDFTLKISRVEAEDVGVYYCFQGSHIPYTFGQGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No.: 14 (human ADM 1-21)
YRQSMNNFQGLRSFGCRFGTC
SEQ ID No.: 15 (human ADM 21-32)
CTVQKLAHQIYQ
SEQ ID No.: 16 (human ADM C-42-52)
CAPRSKISPQGY-CONH₂
SEQ ID No.: 17 (murine ADM 1-19)
YRQSMNQGSRSNGCRFGTC
SEQ ID No.: 18 (murine ADM 19-31)
CTFQKLAHQIYQ
SEQ ID No.: 19 (murine ADM C-40-50)
CAPRNKISPQGY-CONH₂
SEQ ID No.: 20 (mature human Adrenomedullin (mature ADM); amidated ADM; bio-
ADM): amino acids 1-52 or amino acids 95-146 of pro-ADM
YRQSMNNFQGLRSFGCRFGTCTVQKLAHQIYQFTDKDKDNVAPRSKISPQGY-CONH₂
SEQ ID No.: 21 (Murine ADM 1-50)
YRQSMNQGSRSNGCRFGTCTFQKLAHQIYQLTDKDKDGMAPRNKISPQGY-CONH₂
SEQ ID No.: 22 (1-21 of human ADM):
YRQSMNNFQGLRSFGCRFGTC
SEQ ID No.: 23 (1-42 of human ADM):
YRQSMNNFQGLRSFGCRFGTCTVQKLAHQIYQFTDKDKDNVA
SEQ ID No.: 24 (aa 43-52 of human ADM)
PRSKISPQGY-NH₂
SEQ ID No.: 25 (aa 1-14 of human ADM)
YRQSMNNFQGLRSF
SEQ ID No.: 26 (aa 1-10 of human ADM)
YRQSMNNFQG
SEQ ID No.: 27 (aa 1-6 of human ADM)
YRQSMN
SEQ ID No.: 28 (aa 1-32 of human ADM)
YRQSMNNFQGLRSFGCRFGTCTVQKLAHQIYQ
SEQ ID No.: 29 (aa 1-40 murine ADM)
YRQSMNQGSRSNGCRFGTCTFQKLAHQIYQLTDKDKDGMA
SEQ ID No.: 30 (aa 1-31 murine ADM)
YRQSMNQGSRSNGCRFGTCTFQKLAHQIYQL
SEQ ID No.: 31 (proADM: 164 amino acids (22-185 of preproADM))
ARLDVASEF RKKWNKWALS RGKRELRMSS SYPTGLADVK AGPAQTLIRP QDMKGASRSP
EDSSPDAARI RVKRYRQSMN NFQGLRSFGC RFGTCTVQKL AHQIYQFTDK DKDNVAPRSK
ISPQGYGRRR RRSLPEAGPG RTLVSSKPQA HGAPAPPSGS APHFL
SEQ ID NO: 32 (Adrecizumab heavy chain)
QVQLVQSGAEVKKPGSSVKVSCKASGYTFSRYWIEWVRQAPGQGLEWIGEILPGSGSTNYNQ
KFQGRVTITADTSTSTAYMELSSLRSEDTAVYYCTEGYEYDGFDYWGQGTTVTVSSASTKGP
SVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYP
SDIAVEWESNGQPENNYKTTPPVLD
SDGSFFLYSKLTVDKSRWQQGNVFSCSVMHEALHNHYTQKSLSLSPGK
SEQ ID NO: 33 (Adrecizumab light chain)
DVVLTQSPLSLPVTLGQPASISCRSSQSIVYSNGNTYLEWYLQRPGQSPRLLIYRVSNRFSGVP
DRFSGSGSGTDFTLKISRVEAEDVGVYYCFQGSHIPYTFGGGTKLEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID No. 34-human DPP3 (amino acid 1-737)
MADTQYILPNDIGVSSLDCREAFRLLSPTERLYAYHLSRAAWYGGLAVLLQTSPEAPYIYALL
SRLFRAQDPDQLRQHALAEGLTEEEYQAFLVYAAGVYSNMGNYKSFGDTKFVPNLPKEKLE
RVILGSEAAQQHPEEVRGLWQTCGELMFSLEPRLRHLGLGKEGITTYFSGNCTMEDAKLAQD
FLDSQNLSAYNTRLFKEVDGEGKPYYEVRLASVLGSEPSLDSEVTSKLKSYEFRGSPFQVTRG
DYAPILQKVVEQLEKAKAYAANSHQGQMLAQYIESFTQGSIEAHKRGSRFWIQDKGPIVESYI
GFIESYRDPFGSRGEFEGFVAVVNKAMSAKFERLVASAEQLLKELPWPPTFEKDKFLTPDFTS
LDVLTFAGSGIPAGINIPNYDDLRQTEGFKNVSLGNVLAVAYATQREKLTFLEEDDKDLYILW
KGPSFDVQVGLHELLGHGSGKLFVQDEKGAFNFDQETVINPETGEQIQSWYRSGETWDSKFS
TIASSYEECRAESVGLYLCLHPQVLEIFGFEGADAEDVIYVNWLNMVRAGLLALEFYTPEAFN
WRQAHMQARFVILRVLLEAGEGLVTITPTTGSDGRPDARVRLDRSKIRSVGKPALERFLRRLQ
VLKSTGDVAGGRALYEGYATVTDAPPECFLTLRDTVLLRKESRKLIVQPNTRLEGSDVQLLE
YEASAAGLIRSFSERFPEDGPELEEILTQLATADARFWKGPSEAPSGQA
SEQ ID No. 35-human DPP3 (amino acid 474-493 (N-Cys))-immunization peptide
with additional N-terminal Cystein
CETVINPETGEQIQSWYRSGE
SEQ ID No. 36-IGHV1-69*11
QVQLVQSGAEVKKPGSSVKVSCKASGGTFSSYAISWVRQAPGQGLEWMGRIIPILGTANYAQ
KFQGRVTITADESTSTAYMELSSLRSEDTAVYYCARYYYYYGMDVWGQGTTVTVSS
SEQ ID No. 37-HB3
QVQLQQSGAELMKPGASVKISCKATGYTFSRYWIEWVKQRPGHGLEWIGEILPGSGSTNYNE
KFKGKATITADTSSNTAYMQLSSLTSEDSAVYYCTEGYEYDGFDYWGQGTTLTVSS

For the avoidance of doubt, in amino acid sequences as shown herein, C-terminal groups —NH2 and —CONH2 likewise refer to C-terminal amide groups (i.e. C-terminally amidated polypeptides).

1. A method for therapy or prevention of shock in a patient, comprising administering an anti-adrenomedullin (ADM) antibody or anti-ADM antibody fragment or anti-ADM non-Ig scaffold to a patient in need thereof,
wherein said patient has a level of dipeptidyl peptidase 3 (DPP3) which is less than or equal to 40 ng/ml, and
wherein said anti-ADM antibody or anti-ADM fragment or anti-ADM non-Ig scaffold binds to the N-terminal part (amino acid 1-21) of ADM: YRQSMNNFQGLRSFGCRFGTC (SEQ ID No. 14).

2. A method according to claim 1, wherein said shock is selected from the group comprising shock due to hypovolemia, cardiogenic shock, obstructive shock and distributive shock, in particular cardiogenic shock or septic shock.

3. A method according to claim 2, wherein
in case of cardiogenic shock said patient has suffered an acute coronary syndrome or wherein said patient has heart failure, myocarditis, arrhythmia, cardiomyopathy, valvular heart disease, aortic dissection with acute aortic stenosis, traumatic chordal rupture or massive pulmonary embolism, or
in case of hypovolemic shock said patient has suffered a hemorrhagic disease including gastrointestinal bleed, trauma, vascular etiologies and spontaneous bleeding in the setting of anticoagulant use or a non-hemorrhagic disease including vomiting, diarrhea, renal loss, skin losses/insensible losses or third-space loss in the setting of pancreatitis, cirrhosis, intestinal obstruction, trauma, or
in case of obstructive shock said patient has suffered a cardiac tamponade, tension pneumothorax, pulmonary embolism or aortic stenosis, or
in case of distributive shock said patient has septic shock, neurogenic shock, anaphylactic shock or shock due to adrenal crisis.

4. A method according to claim 1, wherein said threshold of DPP3 in a sample of bodily fluid of said patient is ≤30 ng/ml.

5. (canceled)

6. A method according to claim 1, wherein the level of DPP3 in said patient is greater than 22 n/mL.

7. (canceled)

8. A method according to claim 1, wherein either the level of DPP3 protein and/or the level of active DPP3 is determined and compared to a predetermined threshold.

9. A method according to claim 1, wherein the level of DPP3 is determined with an immunoassay, in particular a sandwich immunoassay.

10. A method according to claim 1, wherein said level of DPP3 is DPP3 activity, wherein the method for determining DPP3 activity in a bodily fluid sample of said subject comprises the steps:
contacting said sample with a capture-binder that binds specifically to full-length DPP3,
separating DPP3 bound to said capture binder,
adding substrate of DPP3 to said separated DPP3,
quantifying of said DPP3 activity by measuring and quantifying the conversion of a substrate of DPP3.

11. A method according to claim 1, wherein said patient is additionally has a level of ADM-NH₂ above 40 μg/mL.

12-13. (canceled)

14. A method according to claim 1, wherein the bodily fluid is selected from whole blood, plasma and serum.

15-17. (canceled)

18. A method according to claim 1, wherein said anti-ADM antibody or anti-ADM antibody fragment or anti-ADM non-Ig scaffold recognizes and binds to the N-terminal end (amino acid 1) of ADM-Gly and/or ADM-NH₂.

19. A method according to claim 1, wherein said antibody, antibody fragment or non-Ig scaffold does not bind to the C-terminal portion of ADM, having the sequence amino acid 43-52 of ADM: PRSKISPQGY-NH₂ (SEQ ID NO: 24).

20. A method according to claim 1, wherein said antibody or fragment or scaffold blocks the bioactivity of ADM not more than 80%.

21. A method according to claim 1, wherein said antibody or fragment is a monoclonal antibody or fragment that binds to ADM or an antibody fragment thereof, wherein the heavy chain comprises the sequences:
CDR1:
SEQ ID NO: 1
GYTFSRYW
CDR2:
SEQ ID NO: 2
ILPGSGST
CDR3:
SEQ ID NO: 3
TEGYEYDGFDY
and wherein the light chain comprises the sequences:
CDR1:
SEQ ID NO: 4
QSIVYSNGNTY
CDR2:
RVS
CDR3:
SEQ ID NO: 5
FQGSHIPYT.

22. A method according to claim 1, wherein said antibody or fragment comprises a sequence selected from the group comprising as a VH region:
(AM-VH-C)
SEQ ID NO: 6
QVQLQQSGAELMKPGASVKISCKATGYTFSRYWIEWVKQRPGHGLEWIG
EILPGSGSTNYNEKFKGKATITADTSSNTAYMQLSSLTSEDSAVYYCTE
GYEYDGFDYWGQGTTLTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPK
(AM-VH1)
SEQ ID NO: 7
QVQLVQSGAEVKKPGSSVKVSCKASGYTFSRYWISWVRQAPGQGLEWMG
RILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPK
(AM-VH2-E40)
SEQ ID NO: 8
QVQLVQSGAEVKKPGSSVKVSCKASGYTFSRYWIEWVRQAPGQGLEWMG
RILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPK
(AM-VH3-T26-E55)
SEQ ID NO: 9
QVQLVQSGAEVKKPGSSVKVSCKATGYTFSRYWISWVRQAPGQGLEWMG
EILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPK
(AM-VH4-T26-E40-E55)
SEQ ID NO: 10
QVQLVQSGAEVKKPGSSVKVSCKATGYTFSRYWIEWVRQAPGQGLEWMG
EILPGSGSTNYAQKFQGRVTITADESTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPK
and comprises a sequence selected from the group comprising the following sequence as a VL region:
(AM-VL-C)
SEQ ID NO: 11
DVLLSQTPLSLPVSLGDQATISCRSSQSIVYSNGNTYLEWYLQKPGQSP
KLLIYRVSNRFSGVPDRFSGSGSGTDFTLKISRVEAEDLGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(AM-VL1)
SEQ ID NO: 12
DVVMTQSPLSLPVTLGQPASISCRSSQSIVYSNGNTYLNWFQQRPGQSP
RRLIYRVSNRDSGVPDRFSGSGSGTDFTLKISRVEAEDVGVYYCFQGSH
IPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
(AM-VL2-E40)
SEQ ID NO: 13
DVVMTQSPLSLPVTLGQPASISCRSSQSIVYSNGNTYLEWFQQRPGQSP
RRLIYRVSNRDSGVPDRFSGSGSGTDFTLKISRVEAEDVGVYYCFQGSH
IPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

23. A method according to claim 1, wherein said antibody or fragment comprises the following sequence as a heavy chain:
SEQ ID NO: 32
QVQLVQSGAEVKKPGSSVKVSCKASGYTFSRYWIEWVRQAPGQGLEWIG
EILPGSGSTNYNQKFQGRVTITADTSTSTAYMELSSLRSEDTAVYYCTE
GYEYDGFDYWGQGTTVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKS
LSLSPGK
or a sequence that is >95% identical to it,
and comprises the following sequence as a light chain:
SEQ ID NO: 33
DVVLTQSPLSLPVTLGQPASISCRSSQSIVYSNGNTYLEWYLQRPGQSP
RLLIYRVSNRFSGVPDRFSGSGSGTDFTLKISRVEAEDVGVYYCFQGSH
IPYTFGGGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC
or a sequence that is >95% identical to it.

24. A method according to claim 1, wherein the anti-adrenomedullin (ADM) antibody or anti-ADM antibody fragment or anti-ADM non-Ig scaffold binds to the N-terminal part (amino acid 1-10) of ADM: YRQSMNNFQG (SEQ ID No. 26).

25. A method of therapy according to claim 1, wherein said antibody or fragment or scaffold exhibits a binding affinity to ADM of at least 10-7 M by label-free surface plasmon resonance using a Biacore 2000 system.

26. (canceled)

27. A method according to claim 1, wherein the anti-ADM antibody or anti-ADM antibody fragment is an IgG1 antibody.

28. A method according to claim 1, wherein the anti-adrenomedullin (ADM) antibody or anti-ADM antibody fragment or anti-ADM non-Ig scaffold is in a pharmaceutical composition;
wherein:
said pharmaceutical composition is a solution; or
wherein said pharmaceutical formulation is in a freeze-dried state; or
wherein said pharmaceutical formulation is administered intra-muscular; or
wherein said pharmaceutical formulation is administered intra-vascular; or
wherein said pharmaceutical formulation is administered via infusion; or
wherein said pharmaceutical formulation is to be administered systemically.

29-34. (canceled)
